(19) United States
(12) Patent Application Publication
(10) Pub. No.: US 2021/0283202 A1
(43) Pub. Date: Sep. 16, 2021

(54) E. COLI BACTERIOPHAGE ESC-COP-14 AND USE THEREOF IN INHIBITING GROWTH OF PATHOGENIC E. COLI

(71) Applicant: Intron Biotechnology, Inc., Gyeonggi-do (KR)

(72) Inventors: Seong Jun Yoon, Seoul (KR); Soo Youn Jun, Seoul (KR); Jee Soo Son, Seoul (KR); Hyoung Rok Paik, Incheon (KR); Hyun Joo Lee, Seoul (KR); Sang Hyeon Kang, Seoul (KR)

(21) Appl. No.: 17/258,722

(22) PCT Filed: Jun. 7, 2019
(86) PCT No.: PCT/KR2019/006903

§ 371 Date: Jan. 7, 2021

(30) Foreign Application Priority Data
Jul. 11, 2018 (KR) 10-2018-0080284

(51) Int. Cl. A61K 35/76; A23L 3/3571; A23K 10/16; C12N 7/00

(52) U.S. Cl. CPC A61K 35/76; A23L 3/3571; C12N 2795/10131; C12N 7/00; C12N 2795/10132; A23K 10/16

(57) ABSTRACT

The present invention relates to a Myoviridae bacteriophage Esc-COP-14 (Accession number: KCTC 13528BP) isolated from nature, and characterized by having the ability to specifically kill Escherichia coli and having the genome represented by SEQ. ID. NO: 1, and to a method for preventing and treating the infection of pathogenic Escherichia coli using the composition comprising Myoviridae bacteriophage Esc-COP-14 as an active ingredient.

### TECHNICAL FIELD

The present invention relates to a bacteriophage isolated from nature, which infects Escherichia coli to thus kill Escherichia coli, and a method for preventing and treating the infection of pathogenic Escherichia coli using a composition comprising the same as an active ingredient. More particularly, the present invention relates to a Myoviridae bacteriophage Esc-COP-14 (Accession number: KCTC 13528BP) isolated from nature, which has the ability to specifically kill Escherichia coli and includes the genome represented by SEQ. ID. NO: 1, and a method for preventing the infection of pathogenic Escherichia coli and treating it after the infection using a composition comprising the bacteriophage as an active ingredient.

### BACKGROUND ART

Escherichia coli is a normal flora living in intestinal tracts of human or animals. Usually, Escherichia coli is not pathogenic, but might acquire virulent traits by allogenic and heterogenic transformation. The resulting bacterium incorporating exogenous genes is called as pathogenic Escherichia coli, relative to normal Escherichia coli. This virulent Escherichia coli can cause a lot of diseases, including food poisoning, acute colitis, infection of uninary tract, sepsis and encephalomeningitis. In turn, it can be classified into 5 kinds of microbes, depending upon its pathogenesis, toxicity and the like: Enteropathogenic Escherichia coli (EPEC), Enterotoxigenic Escherichia coli (ETEC), Enterohemorrhagic Escherichia coli (EHEC), Enteroaggregative Escherichia coli (EAEC), Enteroinvasive Escherichia coli (EIEC).

Livestock industries have been afflicted significantly due to the infection of pathogenic Escherichia coli. Therefore, it is necessary to develop a method for preventing this infection and further treating an infectious disease caused by pathogenic Escherichia coli. For this purpose, a variety of antibiotics have been utilized to prevent or treat such a pathogenic infection. However, because of antibiotic abuse, antibiotic-resistant bacterial strains are increasingly observed. Hence, it is urgently required to establish an alternative procedure excluding antibiotics.

Recently, the use of bacteriophages as a countermeasure against bacterial diseases has attracted considerable attention. In particular, interest in bacteriophages is higher than ever due to the preference for environmentally friendly methods. Bacteriophages are very small microorganisms infecting bacteria, and are usually simply called “phages”. Once a bacteriophage infects bacteria, the bacteriophage is proliferated inside the bacterial cell. After proliferation, the progeny of the bacteriophage destroy the bacterial cell wall and escapes from the host bacteria, suggesting that the bacteriophage has the ability to kill bacteria. The manner in which the bacteriophage infects bacteria is characterized by the very high specificity thereof, and thus the number of types of bacteriophages infecting a specific bacterium is limited. That is, a certain bacteriophage can infect only a specific bacterium, suggesting that a certain bacteriophage can kill only a specific bacterium and cannot harm other bacteria. Due to this bacteria specificity of bacteriophages, the bacteriophage confers antibacterial effects only upon target bacteria, but does not affect commensal bacteria in the environment or in animals. Conventional antibiotics, which have been widely used for bacterial treatment, incidentally influence many kinds of bacteria. This causes problems such as environmental pollution and the disturbance of normal microflora in animals. On the other hand, the use of bacteriophages does not disturb normal microflora in animals, because the target bacterium is selectively killed. Hence, the bacteriophage may be utilized safely, which thus greatly lessens the probability of adverse actions in use compared to any other antibiotics.

Bacteriophages were first discovered by the English bacteriologist Twort in 1915 when he noticed that Micrococcus colonies melted and became transparent by something unknown. In 1917, the French bacteriologist d'Herelle discovered that Shigella dysenteriae in the filtrate of dysentery patient feces was melted by something, and further studied this phenomenon. As a result, he independently identified bacteriophages, and named them bacteriophages, which means “eater of bacteria”. Since then, various bacteriophages acting against such pathogenic bacteria as Shigella, Salmonella Typhi, and Vibrio cholerae have been continuously identified.

Owing to the unique ability of bacteriophages to kill bacteria, bacteriophages have attracted anticipation as a potentially effective countermeasure against bacterial infection since their discovery, and there has been a lot of research related thereto. However, since penicillin was discovered by Fleming, studies on bacteriophages have continued only in some Eastern European countries and the former Soviet Union, because the spread of antibiotics was generalized. Since 2000, the limitations of conventional antibiotics have appeared due to the increase in antibiotic-resistant bacteria, and the possibility of developing bacteriophages as a substitute for conventional antibiotics has been highlighted, so that bacteriophages are again attracting attention as antibacterial agents. In particular, recently, government regulations for the use of antibiotics have become more stringent around the world, and thus interest in bacteriophages is increasing and the range of industrial applications therefore is continually broadening.

As demonstrated above, bacteriophages tend to be highly specific for target bacteria. Because of this specificity, bacteriophages frequently exhibit an antibacterial effect only for certain strains of bacteria, even though the bacteria belong to the same species. In addition, the antibacterial strength of bacteriophages may depend on the type of target bacterial strain. Therefore, it is necessary to collect many kinds of bacteriophages that are useful in order to get effective control of specific bacteria. Hence, in order to develop the effective bacteriophage utilization method in response to pathogenic Escherichia coli, many kinds of bacteriophages that exhibit antibacterial action against Escherichia coli must be acquired. Furthermore, the resulting bacteriophages need to be screened as to whether or not they are superior to others from the aspect of antibacterial strength and spectrum.

### DISCLOSURE

### Technical Problem

Therefore, the present inventors endeavored to develop a composition applicable for the prevention or treatment of a pathogenic Escherichia coli infection using a bacteriophage that is isolated from nature and is capable of selectively killing Escherichia coli, and further to establish a method for preventing or treating a pathogenic Escherichia coli infection using the composition. As a result, the present inventors isolated a bacteriophage suitable for this purpose from nature and secured the gene sequence of the genome that distinguishes the isolated bacteriophage from other bacteriophages. Then, the present inventors developed a composition including the bacteriophage as an active ingredient, and identified that this composition is capable of being used to effectively prevent and treat a pathogenic Escherichia coli infection, leading to the completion of the present invention.

Accordingly, an object of the present invention is to provide a Myoviridae bacteriophage Esc-COP-14 (Accession number: KCTC 13528BP) isolated from nature, which has the ability to specifically kill Escherichia coli and which includes the genome represented by SEQ. ID. NO: 1.

Another object of the present invention is to provide a composition applicable for preventing a pathogenic Escherichia coli infection, which includes a bacteriophage Esc-COP-14 infecting Escherichia coli to thus kill Escherichia coli as an active ingredient, and a method for preventing a pathogenic Escherichia coli infection using said composition.

Another object of the present invention is to provide a composition applicable for treating a pathogenic Escherichia coli infection, which includes a bacteriophage Esc-COP-14 infecting Escherichia coli to thus kill Escherichia coli as an active ingredient, and a method for treating a pathogenic Escherichia coli infection using said composition.

Another object of the present invention is to provide a disinfectant for preventing and treating a pathogenic Escherichia coli infection using said composition.

Another object of the present invention is to provide a drinking-water additive for preventing and treating a pathogenic Escherichia coli infection using said composition.

Another object of the present invention is to provide a feed additive effective upon farming by preventing and treating a pathogenic Escherichia coli infection using said composition.

### Technical Solution

The present invention provides a Myoviridae bacteriophage Esc-COP-14 (Accession number: KCTC 13528BP) isolated from nature, which has the ability to specifically kill Escherichia coli and which includes a genome represented by SEQ. ID. NO: 1, and a method for preventing and treating a pathogenic Escherichia coli infection using a composition including the same as an active ingredient.

The bacteriophage Esc-COP-14 was isolated by the present inventors and then deposited at Korea Collection for Type Cultures, Korea Research Institute of Bioscience and Biotechnology on May 18, 2018 (Accession number: KCTC 13528BP).

The present invention also provides a disinfectant, a drinking-water additive, and a feed additive applicable for the prevention or treatment of the infection of pathogenic Escherichia coli, which include the bacteriophage Esc-COP-14 as an active ingredient.

Since the bacteriophage Esc-COP-14 included in the composition of the present invention kills pathogenic Escherichia coli efficiently, it is considered effective in the prevention (prevention of infection) or treatment (treatment of infection) of diseases caused by pathogenic Escherichia coli. Therefore, the composition of the present invention is capable of being utilized for the prevention and treatment of diseases caused by pathogenic Escherichia coli.

In this description, the terms “prevention” and “prevent” indicate (i) to block the infection of pathogenic Escherichia coli by protecting a host from its invasion or by inhibiting the proliferation of pathogens after the Escherichia coli infection; and (ii) to suppress the progression of diseases caused by the pathogenic Escherichia coli.

In this description, the terms “treatment” and “treat” indicate all actions that (i) suppress diseases caused by pathogenic Escherichia coli; or (ii) alleviate the pathological condition of the diseases caused by the pathogenic Escherichia coli.

In this description, the terms “isolate”, “isolating”, and “isolated” indicate actions which isolate bacteriophages from nature by applying diverse experimental techniques and which secure characteristics that can distinguish the target bacteriophage from others, and further include the action of proliferating the target bacteriophage using bioengineering techniques so that the target bacteriophage is industrially applicable.

The pharmaceutically acceptable carrier included in the composition of the present invention is one that is generally used for the preparation of a pharmaceutical formulation, and examples thereof include lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methylcellulose, methylhydroxybenzoate, propylhydroxybenzoate, talc, magnesium stearate, and mineral oil, but are not limited thereto. The composition of the present invention may additionally include lubricants, wetting agents, sweeteners, flavors, emulsifiers, suspending agents, and preservatives, in addition to the above ingredients.

In the composition of the present invention, the bacteriophage Esc-COP-14 is included as an active ingredient. The bacteriophage Esc-COP-14 is included at a concentration of 1×10¹ pfu/ml to 1×10³⁰ pfu/ml or 1×10¹ pfu/g to 1×10³⁰ pfu/g, and preferably at a concentration of 1×10⁴ pfu/ml to 1×10¹⁵ pfu/ml or 1×10⁴ pfu/g to 1×10¹⁵ pfu/g.

The composition of the present invention can be formulated according to a method that can be easily performed by those of ordinary skill in the art to which the present invention pertains using a pharmaceutically acceptable carrier and/or excipient in the form of a unit dose or in a multi-dose container. Then, the formulation may be in the form of a solution, suspension, or emulsion in oil or a water-soluble medium, extract, powder, granule, tablet, or capsule. A dispersing agent or stabilizer may be additionally included.

The composition of the present invention may be prepared as disinfectant, drinking-water additive or feed additive according to its purpose, without limitation thereto.

In order to improve the effectiveness of above purpose, bacteriophages that confer antibacterial activity against other bacterial species may be further included in the composition of the present invention. In addition, other kinds of bacteriophages that have antibacterial activity against Escherichia coli may be further included in the composition of the present invention. These bacteriophages may be combined properly so as to maximize antibacterial effects, because their antibacterial activities against Escherichia coli may be different from the aspects of antibacterial strength and spectrum.

### Advantageous Effects

The method for preventing and treating an infectious disease caused by the pathogenic Escherichia coli, using the composition comprising the bacteriophage Esc-COP-14 as an active ingredient according to the present invention has advantages to be highly specific for Escherichia coli, compared to conventional methods based upon existing antibiotics and the like. This means that the composition can be used to prevent or treat infectious diseases caused by this Escherichia coli without affecting other commensal bacteria, and further have less side effects attributable to the use thereof. Unfortunately, universal antibiotics result in influencing upon normal microflora of animals and weakening their immunity, which entails various adverse actions owing to the use thereof. Meanwhile, bacteriophages can manifest a distinguished activity against the same bacterial species, with regard to their antibacterial strength and spectrum [the spectrum exerting the antibacterial effect of bacteriophages upon several bacterial strains belonging to Escherichia coli. Typically, bacteriophages are effective exclusively upon certain bacterial strains of the same bacterial species. That is to say, their susceptibility against bacteria depends on individual strains among the same bacterial species]. Accordingly, the present invention can provide a differential antibacterial activity of the bacteriophage against pathogenic Escherichia coli, compared to that of any other bacteriophages. In practice, this may confer a remarkable merit in the industrial field.

### DESCRIPTION OF DRAWINGS

FIG. 1 shows the electron micrograph of the bacteriophage Esc-COP-14.

FIG. 2 shows the experimental result of the activity of bacteriophage Esc-COP-14 killing Escherichia coli. Culture media containing Bacteriophage Esc-COP-14 is spread onto the right side of agar plate and control buffer without bacteriophages is spotted onto the left side. As observed in the right side, the resulting clear zone reveals a plaque formed after the lysis of test bacteria due to Bacteriophage Esc-COP-14.

### MODE FOR INVENTION

Hereinafter, the present invention will be described in detail with reference to Examples. However, the Examples are merely examples of the present invention, and the scope of the present invention is not limited to.

### Example 1: Isolation of Bacteriophage Capable of Killing Escherichia coli

Samples were collected from a natural environment to isolate bacteriophages capable of killing Escherichia coli. Herein, the Escherichia coli strains selected for the bacteriophage isolation has been gratefully donated from ATCC (American Type Culture Collection; ATCC43894).

The procedure for isolating the bacteriophage is described in detail hereinafter. The collected sample was added to a TSB (Tryptic Soy Broth) culture medium (casein digest, 17 g/L; soybean digest, 3 g/L; dextrose, 2.5 g/L; NaCl, 5 g/L; dipotassium phosphate, 2.5 g/L) inoculated with Escherichia coli at a ratio of 1/1000, followed by shaking culture at 37° C. for 3 to 4 hours. Upon completion of the culture, centrifugation was performed at 8,000 rpm for 20 minutes and a supernatant was recovered. The recovered supernatant was inoculated with Escherichia coli at a ratio of 1/1000, followed by shaking culture at 37° C. for 3 to 4 hours. When the sample contained the bacteriophage, the above procedure was repeated 5 times in order to sufficiently increase the number (titer) of the bacteriophage. After repeating the procedure 5 times, the culture solution was subjected to centrifugation at 8,000 rpm for 20 minutes. After the centrifugation, the recovered supernatant was filtered using a 0.45 μm filter. The obtained filtrate was used in a typical spot assay for examining whether or not a bacteriophage capable of killing Escherichia coli was included therein.

The spot assay was performed as follows: TSB culture medium was inoculated with Escherichia coli at a ratio of 1/1000, followed by shaking culture at 37° C. overnight. 3 ml (OD₆₀₀ of 1.5) of the culture medium of Escherichia coli prepared above was spread on TSA (casein digest, 15 g/L; soybean digest, 5 g/L; NaCl, 5 g/L; agar, 15 g/L) plate. The plate was left on a clean bench for about 30 minutes to dry the spread solution. After drying, 10 μl of the prepared filtrate was spotted onto the plate culture medium on which Escherichia coli was spread and then left to dry for about 30 minutes. After drying, the plate culture medium that was subjected to spotting was stationary-cultured at 37° C. for one day, and then examined for the formation of clear zones at the positions where the filtrate was dropped. In the case of the filtrate generated a clear zone, it is judged that the bacteriophage capable of killing Escherichia coli was included therein. Through the above examination, the filtrate containing the bacteriophage having the ability to kill Escherichia coli could be obtained.

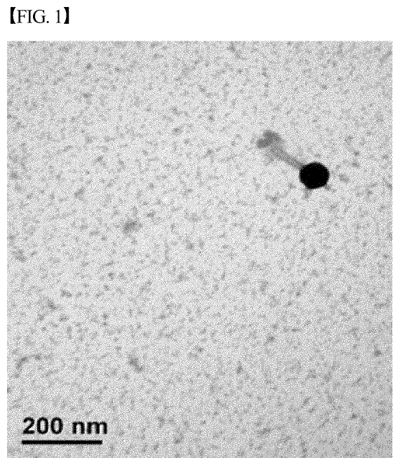

The pure bacteriophage was isolated from the filtrate confirmed above to have the bacteriophage capable of killing Escherichia coli. A conventional plaque assay was used to isolate the pure bacteriophage. In detail, a plaque formed in the course of the plaque assay was recovered using a sterilized tip, which was then added to the culture solution of Escherichia coli, followed by culturing at 37° C. for 4 to 5 hours. After the culturing, centrifugation was performed at 8,000 rpm for 20 minutes to obtain a supernatant. The Escherichia coli culture solution was added to the obtained supernatant at a volume ratio of 1/50, followed by culturing at 37° C. for 4 to 5 hours. In order to increase the number of bacteriophages, the above procedure was repeated at least 5 times. Then, centrifugation was performed at 8,000 rpm for 20 minutes in order to obtain the final supernatant. A plaque assay was further performed using the resulting supernatant. In general, the isolation of a pure bacteriophage is not completed through a single iteration of a procedure, so the above procedure was repeated using the resulting plaque formed above. After at least 5 repetitions of the procedure, the solution containing the pure bacteriophage was obtained. The procedure for isolating the pure bacteriophage was generally repeated until the generated plaques became similar to each other in size and morphology. In addition, final isolation of the pure bacteriophage was confirmed using electron microscopy. The above procedure was repeated until the isolation of the pure bacteriophage was confirmed using electron microscopy. The electron microscopy was performed according to a conventional method. Briefly, the solution containing the pure bacteriophage was loaded on a copper grid, followed by negative staining with 2% uranyl acetate and drying. The morphology thereof was then observed using a transmission electron microscope. The electron micrograph of the pure bacteriophage that was isolated is shown in FIG. 1. Based on the morphological characteristics, the novel bacteriophage isolated above was confirmed to belong to the Myoviridae bacteriophage.

The solution containing the pure bacteriophage confirmed above was subjected to the following purification process. The Escherichia coli culture solution was added to the solution containing the pure bacteriophage at a volume ratio of 1/50 based on the total volume of the bacteriophage solution, followed by further culturing for 4 to 5 hours. After the culturing, centrifugation was performed at 8,000 rpm for 20 minutes to obtain a supernatant. This procedure was repeated 5 times in order to obtain a solution containing sufficient numbers of the bacteriophage. The supernatant obtained from the final centrifugation was filtered using a 0.45 μm filter, followed by a conventional polyethylene glycol (PEG) precipitation process. Specifically, PEG and NaCl were added to 100 ml of the filtrate until reaching 10% PEG 8000/0.5 M NaCl, and then left at 4° C. for 2 to 3 hours. Thereafter, centrifugation was performed at 8,000 rpm for 30 minutes to obtain the bacteriophage precipitate. The resulting bacteriophage precipitate was suspended in 5 ml of a buffer (10 mM Tris-HCl, 10 mM MgSO₄, 0.1% gelatin, pH 8.0). The resulting material was referred to as a bacteriophage suspension or bacteriophage solution.

As a result, the pure bacteriophage purified above was collected, was named the bacteriophage Esc-COP-14, and then deposited at Korea Collection for Type Culture, Korea Research Institute of Bioscience and Biotechnology on May 18, 2018 (Accession number: KCTC 13528BP).

### Example 2: Separation and Sequence Analysis of Genome of Bacteriophage Esc-COP-14

The genome of the bacteriophage Esc-COP-14 was separated as follows. The genome was separated from the bacteriophage suspension obtained using the same method as in Example 1. First, in order to remove DNA and RNA of Escherichia coli included in the suspension, 200 U of each of DNase I and RNase A was added to 10 ml of the bacteriophage suspension and then left at 37° C. for 30 minutes. After being left for 30 minutes, in order to stop the DNase I and RNase A activity, 500 μl of 0.5 M ethylenediaminetetraacetic acid (EDTA) was added thereto and then left for 10 minutes. In addition, the resulting mixture was further left at 65° C. for 10 minutes, and 100 μl of proteinase K (20 mg/ml) was then added thereto so as to break the outer wall of the bacteriophage, followed by reaction at 37° C. for 20 minutes. After that, 500 μl of 10% sodium dodecyl sulfate (SDS) was added thereto, followed by reaction at 65° C. for 1 hour. After reaction for 1 hour, 10 ml of the mixed solution of phenol:chloroform:isoamyl alcohol, mixed at a component ratio of 25:24:1, was added to the reaction solution, followed by mixing thoroughly. In addition, the resulting mixture was subjected to centrifugation at 13,000 rpm for 15 minutes to separate layers. Among the separated layers, the upper layer was selected, and isopropyl alcohol was added thereto at a volume ratio of 1.5, followed by centrifugation at 13,000 rpm for 10 minutes in order to precipitate the genome. The precipitate was recovered and washed by addition of 70% ethanol, then followed by centrifugation at 13,000 rpm for 10 minutes. The washed precipitate was recovered, vacuum-dried and then dissolved in 100 μl of water. This procedure was repeated to obtain a sufficient amount of the genome of the bacteriophage Esc-COP-14.

Information on the sequence of the genome of the bacteriophage Esc-COP-14 obtained above was secured by performing next-generation sequencing analysis with Pac-bio equipment in the National Instrumentation Center for Environmental Management, Seoul National University. Finally, the genome of the bacteriophage Esc-COP-14 has a size of 150,995 bp and whole genome sequence is set forth in SEQ. ID. NO: 1.

The homology (similarity) of the bacteriophage Esc-COP-14 genomic sequence obtained above is compared to previously reported sequences of bacteriophage genome and investigated using BLAST on the web. As a result, the genomic sequence of the bacteriophage Esc-COP-14 was found relatively high homologous with that of Escherichia coli bacteriophage ESC05 (Genbank Accession No. KX664695.2) (query coverage: 95%, sequence identity: 98%). In contrast, the bacteriophage Esc-COP-14 has a circular genome and Escherichia coli bacteriophage ESC05 has a linear genome. Further, it is possibly concluded that both the bacteriophages are genetically distinguished, since the bacteriophage Esc-COP-14 genome has 269 open reading frames (ORF) compared to 275 ORFs in the Escherichia coli bacteriophage ESC05. Moreover, it is elucidated that tail-associated proteins such as tail fiber, tail tip and the like play an important role to invade bacteria and exert antibacterial activity. Particularly, the genetic difference between these proteins contributes to characteristics of bacteriophages, relative to that of other proteins within the genome. For example, it has been reported that 2 subject bacteriophages having a similar genomic structure except for one amino acid of their tail fiber (phiEF24C: NCBI Accession No. AP009390.1, phiEF24C-P2: NCBI Accession No. AB609718.1) revealed distinct antibacterial spectrums (different morbidity and susceptibility) (PLoS One. 2011; 6(10):e26648). The tail fiber proteins of both the bacteriophages are different from each other in one 1287^{th} amino acid, among total 1825 amino acids.

On the other hand, both bacteriophage Esc-COP-14 and bacteriophage ESC05 are also identified to include 2 genes encoding the tail fiber protein within their genomes, located in each discriminable arrangement. Furthermore, the amino acid sequences of both the bacteriophages have been analyzed and compared. As a consequence, it is confirmed that the amino acid sequence of the tail fiber protein should be remarkably different in both bacteriophage Esc-COP-14 and bacteriophage ESC05 (the former amino acid sequence of the tail fiber protein contains among total 674 amino acids, 7 amino acids different from each other, the latter amino acid sequence of the tail fiber protein contains among total 958 amino acids, 9 amino acids displaced).

Such a genetic feature discriminated between both bacteriophage Esc-COP-14 and bacteriophage ESC05 results in a variety of apparent and functional traits distinct in both the bacteriophages. Further, it is obvious that this genetic difference from both bacteriophages should influence upon their industrial applications.

Based upon this result, it can be concluded that the bacteriophage Esc-COP-14 must be a novel bacteriophage distinguished from previously reported bacteriophages. Further, since the antibacterial strength and spectrum of bacteriophages typically depend on the type of bacteriophage, it is considered that the bacteriophage Esc-COP-14 can provide antibacterial activity different from that of any other bacteriophages reported previously.

### Example 3: Evaluation of Ability of Bacteriophage Esc-COP-14 to Kill Pathogenic Escherichia coli

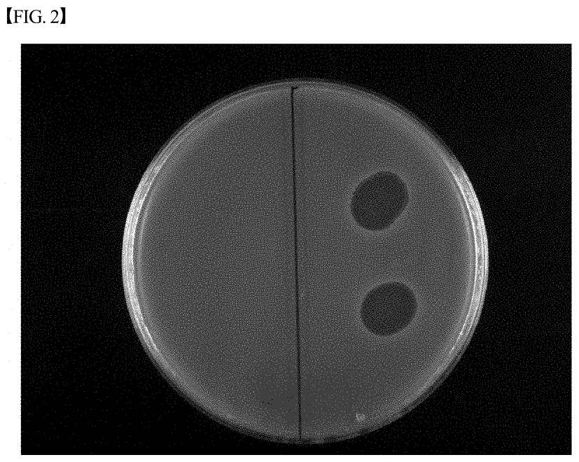

The ability of the isolated bacteriophage Esc-COP-14 to kill Escherichia coli was evaluated. In order to evaluate the killing ability, the formation of clear zones was observed using the spot assay in the same manner as described in Example 1. Total 10 strains have been donated from ATCC or isolated to be identified as pathogenic Escherichia coli by the present inventors and then utilized as Escherichia coli for evaluating its killing ability. The bacteriophage Esc-COP-14 has killed total 9 strains including ATCC43894 strain among 10 Escherichia coli strains for this experiment. The representative experimental result is shown in FIG. 2. Meanwhile, the ability of the bacteriophage Esc-COP-14 to kill Bordetella bronchiseptica, Enterococcus faecalis, Enterococcus faecium, Streptococcus mutans, Streptococcus uberis and Pseudomonas aeruginosa was also investigated in a separate experiment. As a result, the bacteriophage Esc-COP-14 did not kill these microorganisms.

Based on above results, it is confirmed that the bacteriophage Esc-COP-14 has the outstanding activity to kill Escherichia coli and a broad antibacterial spectrum comparing with previously reported bacteriophages against Escherichia coli, suggesting that the bacteriophage Esc-COP-14 can be used as an active ingredient for the composition preventing and treating diseases caused by Escherichia coli.

### Example 4: Prevention of Pathogenic Escherichia coli Infection Using Bacteriophage Esc-COP-14

Herein, the terms “prevention” and “prevent” refer to block the infection of pathogenic Escherichia coli by protecting a host from its invasion or by suppressing the proliferation of this pathogens after the infection, or to inhibit the progression of diseases caused by the infected E. coli as described above. Therefore, in order to provide this “preventive” effect, the bacteriophage should possibly suppress bacterial division or kill Escherichia coli too reduce its number. In this example, the bacteriophage Esc-COP-14 of the present invention has been examined whether it satisfied this requirement or not.

100 μl of a bacteriophage Esc-COP-14 solution at 1×10⁸ pfu/ml was added to a tube containing 9 ml of a TSB culture medium. To another tube containing 9 ml of a TSB culture medium, only the 100 μl of TSB culture medium was further added. A pathogenic Escherichia coli culture solution was then added to each tube so that absorbance reached about 0.5 at 600 nm. After pathogenic Escherichia coli was added, the tubes were transferred to an incubator at 37° C., followed by shaking culture, during which the growth of pathogenic Escherichia coli was observed. As presented in Table 1, it was observed that the proliferation of pathogenic Escherichia coli was inhibited in the tube to which the bacteriophage Esc-COP-14 solution was added, while the proliferation of pathogenic Escherichia coli was not inhibited in the tube to which the bacteriophage solution was not added.

TABLE 1
Growth inhibition of pathogenic Escherichia coli

| | OD₆₀₀ absorbance value | | |
|---|---|---|---|
| | 0 minutes | 30 minutes | 60 minutes |
| Classification | after culture | after culture | after culture |
| Bacteriophage solution | 0.5 | 0.7 | 1.9 |
| is not added | | | |
| Bacteriophage solution | 0.5 | 0.3 | 0.1 |
| is added | | | |

The above results indicate that the bacteriophage Esc-COP-14 of the present invention not only suppresses the proliferation of pathogenic Escherichia coli but also has the ability to kill pathogenic Escherichia coli. Therefore, it is concluded that the bacteriophage Esc-COP-14 can be used as an active ingredient of the composition for preventing a pathogenic Escherichia coli infection.

### Example 5: Animal Test for Prevention of Diseases Caused by Pathogenic Escherichia coli Using Bacteriophage Esc-COP-14

Herein, the terms “prevention” and “prevent” refer to block the infection of pathogenic Escherichia coli by protecting a host from its invasion or by suppressing the proliferation of this pathogens after the infection, or to inhibit the progression of diseases caused by the infected E. coli as described above. Relating to this, in this example, the bacteriophage Esc-COP-14 of the present invention has been examined whether it can be utilized for the prevention to suppress the progression of diseases caused by the pathogenic Escherichia coli after being invaded.

The preventive effect of the bacteriophage Esc-COP-14 on weaning piglets afflicted with pathogenic Escherichia coli was investigated. Total 2 groups of four 25-day-old weaning piglets per group were prepared and reared separately in experimental farming pig pens (1.1 m×1.0 m), and the experiment was conducted for 14 days. The environment surrounding the pens under the warming facility was controlled, and the temperature and humidity in the pig pens were maintained consistent, and the floor of the pig pen was cleaned every day. From the day starting the experiment until the day completing it, for experimental groups (groups feeding with the bacteriophage), pigs were fed with the bacteriophage Esc-COP-14 at 1×10⁸ PFU/g according to a conventional procedure. In contrast, pigs for a control group (a group feeding without the bacteriophage) were fed without the bacteriophage Esc-COP-14 from the starting day until the completing day of the experiment, according to the same procedure. From the 7^{th} day after starting the experiment, for both experimental groups (groups feeding with bacteriophage) and control group (a group feeding without bacteriophage), all pigs were fed twice a day for 2 days with the pathogenic Escherichia coli at 1×10⁸ cfu/g so as to induce the infection. Diarrhea was examined in all test animals on a daily basis after feeding with the pathogenic Escherichia coli (from the 7^{th} day after starting the experiment). The extent of diarrhea was determined by measuring according to a diarrhea index. The diarrhea index was measured using a commonly used Fecal Consistency (FC) score (normal: 0, soft stool: 1, loose diarrhea: 2, severe diarrhea: 3). The results are shown in Table 2.

TABLE 2
diarrhea index (average value)

| classification | D7 | D8 | D9 | D10 | D11 | D12 | D13 | D14 |
|---|---|---|---|---|---|---|---|---|
| Control group (feeding | 1.4 | 2.0 | 1.5 | 1.4 | 1.5 | 1.4 | 1.1 | 1.1 |
| without bacteriophage) | | | | | | | | |
| Experimental group | 0.7 | 0.3 | 0.3 | 0 | 0 | 0 | 0 | 0 |
| (feeing with | | | | | | | | |
| bacteriophage) | | | | | | | | |

From the above results, it is confirmed that the bacteriophage Esc-COP-14 of the present invention could be potentially effective to prevent diseases caused by pathogenic Escherichia coli.

### Example 6: Treatment of Infectious Diseases of Pathogenic Escherichia coli Using Bacteriophage Esc-COP-14

The therapeutic effect of the bacteriophage Esc-COP-14 on pigs afflicted with pathogenic Escherichia coli was investigated. Total 2 groups of four 25-day-old weaning piglets per group were prepared and reared separately in experimental farming pig pens (1.1 m×1.0 m), and the experiment was performed for 14 days. The environment surrounding the pens under the warming facility was controlled, and the temperature and humidity in the pig pens were maintained constant, and the floor of the pig pen was cleaned every day. On the 7^{th} day after the start of the experiment, all pigs were orally administered with a pathogenic Escherichia coli solution using an oral injection tube. The administered pathogenic Escherichia coli solution was prepared as follows. Pathogenic Escherichia coli was cultured at 37° C. for 18 hours using a TSB culture medium, after which the bacteria were isolated and adjusted to 1×10⁹ CFU/ml using physiological saline (pH 7.2). From the day following administration of the pathogenic Escherichia coli, the bacteriophage Esc-COP-14 of 1×10⁹ PFU was orally administered to the pigs in the experimental group (bacteriophage solution-administered group) twice a day in the same manner as the administration of the pathogenic Escherichia coli solution. The pigs in the control group (the group not administered with bacteriophage solution) were not subjected to any treatment. Feed and drinking water were provided to both the control and experimental groups. Diarrhea was examined in all test animals on a daily basis after administration of the pathogenic Escherichia coli. The extent of diarrhea was determined by measuring according to a diarrhea index. The diarrhea index was measured using a commonly used Fecal Consistency (FC) score (normal: 0, soft stool: 1, loose diarrhea: 2, severe diarrhea: 3). The results are shown in Table 3.

TABLE 3
Result of measurement of diarrhea index

| Days after | | | | | | | |
|---|---|---|---|---|---|---|---|
| administration with | | | | | | | |
| pathogenic E. coli | 0 | 1 | 2 | 3 | 4 | 5 | 6 |
| Control group | 1.0 | 1.25 | 1.25 | 1.25 | 1.0 | 0.75 | 0.75 |
| (bacteriophage | | | | | | | |
| solution not administered) | | | | | | | |
| Experimental group | 0.5 | 0.25 | 0.25 | 0.25 | 0.25 | 0 | 0 |
| (administered with | | | | | | | |
| bacteriophage solution) | | | | | | | |

From the above results, it is confirmed that the bacteriophage Esc-COP-14 of the present invention could be also potentially effective in the treatment of infectious diseases caused by pathogenic Escherichia coli.

### Example 7: Preparation of Feed Additives and Feed

Feed additives were prepared using bacteriophage Esc-COP-14 solution so that the bacteriophage Esc-COP-14 was contained in an amount of 1×10⁹ pfu per 1 g of the feed additives. The method of preparing the feed additives was as follows: Maltodextrin (50%, w/v) was added to the bacteriophage solution, and the resulting mixture was then freeze-dried. Finally, the dried mixture was ground into fine powder. In the above-described preparation procedure, the drying procedure can be replaced with drying under reduced pressure, drying with heat, or drying at room temperature. In order to prepare the control for comparison, the feed additives that did not contain the bacteriophage but contained a buffer (10 mM Tris-HCl, 10 mM MgSO₄, 0.1% gelatin, pH 8.0) used to prepare the bacteriophage solution was prepared.

The two kinds of feed additives thus prepared were each mixed with a pig-based feed at a weight ratio of 1/1000, thus ultimately preparing two kinds of feed.

### Example 8: Preparation of Drinking-Water Additives and Disinfectants

Drinking-water additives and disinfectants were prepared in the same manner because they differ only in utilization and are the same in its formulation. The drinking-water additives (or disinfectants) were prepared using bacteriophage Esc-COP-14 solution so that the bacteriophage Esc-COP-14 was contained in an amount of 1×10⁹ pfu per 1 ml of the drinking-water additives (or disinfectants). In the method of preparing the drinking-water additives (or disinfectants), the bacteriophage Esc-COP-14 solution was added so that the bacteriophage Esc-COP-14 was contained in an amount of 1×10⁹ pfu per 1 ml of the buffer used for preparing the bacteriophage solution, and mixed sufficiently. For a control group, the same buffer for the bacteriophage solution was used as the drinking-water additive (or disinfectant) that did not contain the bacteriophage.

These two kinds of drinking-water additives (or disinfectants) prepared above were diluted with water at a volume ratio of 1/1,000, thus ultimately preparing drinking-water additives (or disinfectants).

### Example 9: Confirmation of Feeding Effect on Pig Farming

Improvement in pig farming as the result of feeding was investigated using the feed, drinking water or disinfectant prepared in Examples 7 and 8. In particular, the evaluation was focused on mortality ratio. Total 30 piglets were divided into three groups, each including 10 piglets (group A: fed with the feed, group B: fed with the drinking water, and group C: treated with the disinfectant), and an experiment was performed over four weeks. Each group was divided into sub-groups each including 5 piglets, and the sub-groups were classified into a sub-group to which the bacteriophage Esc-COP-14 was applied (sub-group-\textcircled{1}) and a sub-group to which the bacteriophage was not applied (sub-group-\textcircled{2}). In the present experiment, the target piglets were 20-day-old weaning piglets, and the piglets of the experimental sub-groups were farmed in separate pens placed apart from each other at a certain space interval. The sub-groups were classified and named as shown in Table 4 below.

TABLE 4
Sub-group classification and expression in pig feeding experiment

| | Sub-group classification and expression | |
|---|---|---|
| | Bacteriophage Esc-COP-14 | Bacteriophage is not |
| Application | is applied | applied |
| Group fed with feed | A-\textcircled{1} | A-\textcircled{2} |
| Group fed with | B-\textcircled{1} | B-\textcircled{2} |
| drinking water | | |
| Group treated with | C-\textcircled{1} | C-\textcircled{2} |
| disinfectant | | |

In the case of provision of the feed, the feed prepared in Example 7 was provided according to a conventional feeding method as classified in Table 4, and the drinking water prepared in Example 8 was provided according to a conventional drinking-water feeding method as classified in Table 4. In the case of disinfection, the disinfection was carried out alternately with the pre-existing disinfection 3 times a week. Disinfection using a typical disinfectant was not performed on the day at which the disinfectant of the present invention was sprayed. The experimental results are shown in Table 5 below.

TABLE 5

| Group | Mortality (%) |
|---|---|
| A-\textcircled{1} | 0 |
| A-\textcircled{2} | 40 |
| B-\textcircled{1} | 0 |
| B-\textcircled{2} | 60 |
| C-\textcircled{1} | 0 |
| C-\textcircled{2} | 60 |

The above results indicate that the provision of the feed and the drinking water prepared according to the present invention and the disinfection according to the present invention were effective in reducing mortality ratio upon pig farming. Therefore, it is concluded that the composition of the present invention is capable of being effectively applied to improving the results of pig feeding.

While the present invention has been particularly shown and described with reference to exemplary embodiments thereof, those skilled in the art will appreciate that the specific description is only a preferred embodiment, and that the scope of the present invention is not limited thereto. It is therefore intended that the scope of the present invention be defined by the claims appended hereto and their equivalents.

[Accession number]

Name of Depositary Authority: KCTC

Accession number: KCTC 13528BP

Accession date: 20180518

1. A Myoviridae bacteriophage Esc-COP-14 (Accession number: KCTC 13528BP) isolated from nature, which has an ability to specifically kill Escherichia coli and which includes a genome expressed by SEQ. ID. NO: 1.

2. A composition for preventing and treating a pathogenic Escherichia coli infection, comprising:
the bacteriophage Esc-COP-14 (Accession number: KCTC 13528BP) of claim 1 as an active ingredient.

3. The composition for preventing and treating the pathogenic Escherichia coli infection of claim 2, wherein the composition is used to prepare a feed additive, a drinking-water additive, or a disinfectant.

4. A method for preventing or treating a pathogenic Escherichia coli infection, the method comprising:
administering to an animal other than a human the composition including the bacteriophage Esc-COP-14 (Accession number: KCTC 13528BP) of claim 2 as an active ingredient.

5. The method for preventing or treating the pathogenic Escherichia coli infection of claim 4, wherein the composition is administered to the animal other than the human for use of a feed additive, a drinking-water additive, or a disinfectant.


SEQUENCE LISTING (1 sequence)

SEQ ID NO: 1 - DNA, 150995 bp, Bacteriophage Esc-COP-14
acgcgaattttcgggccgtcaggtccacgcagcgacaattcccaaatatcatgcgaatgagaataattatcattcgtattcaatgacttatgcaaggcttatgccaacatagcgacaaggtgggccacttgattataaatcacacaaacacttatttatcaatggtttggcaacttcatcatatgacggaacattgtcaactgatcccgctcatgacaagcagactattatttatatgagaatgattattatttaaacgtttattaacataaaaactaaaataaatcaagattattctgattttatttacgtttaagagaagatgctcaacatatcgcgtagcgatagtattcgcccccatgatatttgctcttctcttagtgctcaacattcccgcctgtggcggtaacatttcgcccccaaaagtattgccttttattctattatgtgcatagtgtgcaatgtatcggaatgatgataatcatcctcccattgcccccataattattgccatgctaatgatttcatcttgtaattaccacaatgattattgttattgcgctaatgttatgatcttggtaattacatataatgctaataataagccagcgcaataagtgtaagcgtcattacgcttgcaattattgcaagctacgcagtaacaagaaataaccatcaccgatggttatgattgttggggtgtagttggcttattattagctcgctatgtgtccggcatagcgtgctaatgatgatcctgtgaatgattcgttttgtaatgatttcattcaggcactattttgctaatagttgttattttaatggtttcgtttgttattgttcgttagttggttgttttgtcgtgttatcgttatcttgcttagtattcatcatggtttgattgtttgtcgttatcaatcatcttgctattattcctttgattatgattagtttgcttattgttttaggttgttggttagttagttgttgcgaactattatcaacattatagttatgcacttatattgcatagataaacatggatagatgataatggttatcattgcactagaaaggaggtaaaagggtgtttttgatggtttgttgttggttgttgatcgattttaaccgctctaaatggctctctaacagcctgttaccctgggaggtatggttattcgtttaatggttgattagctcgataccgctatttattcagttttgttctgtgtctgttttccataggcggaatctatcaacattattcaggcaaaaagaaagggcgcttggtgcgccccttgttttgttgtcaattataacgcttcatagattgagtagatccggtttgccaggtcattacgaaagcggaatttttcatcacgtttaatcatcagatccttaaaaccgcattctttcaaggcttcattaactttatggcggcaaacgttcatattatcctgcgcgtcaccaataaacttattactatgatgcattgctttgatctcttcctctaataccagggcaactacacggaccggatcaagctgatatgcggtaacatccatgcggcattgtaaacggcttttaacatcgtttaccggatatactgcaacctcttccccgtcaacgttcacgataaccaatttaccatcgatttcgaagcctttggcggcgctaccgtgacaaaaagcgttatcgaaggagtaagctggattatttgatttaatgttcatggtgttgtcctttgtttcgtttcgttgtttgctgccagtgagttaaatataatataacgccgatcccctgtcaataggggatagcgcattatttacatttctttacgttaaagggcgcttagtgcgccccttgtataatcttactttgtgcggttgtgaacgttatcgcgttcaaccatcagggcaaggcggatcagttgatcagttgtatcgcgtaaattgtcgggccatgttttactgttccagctatccaggtttgattgtatataggtttcaatggcttgatccatattatcaaacaagttttcctccatgctctcggctgtttcacactggaaagcattagcgacactattaatcaataagcaagccagtaaatcagcattacgccaatttttacggatagtttcatctgtcagataaccaacgagattaacctttgcgccagtggttgacgttgcgatcacttcttgcgctttttcaactgctttaattgctgatttgctcatcttcttttcctctttgtgggcttgctgtattgctcgccctacgtgataaataataatgtaagcgtgatccggtgtcaataggtggaattgtaaagatttgatagtttatctctttgatgttttgttgtttgataggtgttagctatcaatatatagcggggtcttccggtgattatcttcccctgtcgccaggtgccgcccgttcaataagcataacaaaaaacaggacaaaagcaagccctgtttttgtaaagattagatgatctgcatatcctccacgataatttctaatttccaaacaagatgatctaagctccctgataaaaatgattctgatacataaccgccatcggacaccgtagcaaggatctcgccatttgcttgctctttgatttgaacatccaggcggttagcctttgctttattcaggacggcgatcaggcggttagctgtagtcatggtgttttccttccagttgtgagcggcaccattgccgccctacgaaatacatcttacactatccggcacatcgtgcaagctgttttatacttcctcacctataaagtcggatctgttcttctctgtcatacgtttaagcaattcttgcatgtggcttatagcttgcaatgtgtcttcttccaggcttgaatccacataatcaggatcattctttaccttgctatggttgtgataccgcaacgctgctaatacctttctaacttgttctgtattcattttattattccttaaaaccaggtgccaaccgtttccaataagtcactgactaccaggacaacactaatcagcacatattcaaccaggaaggggatcagcgccagcgttgctactatgatccccattgtcatgattacctttctcattgttggcggtttacgtaagctttaccgtttacggtcatcggtatggcctcaaggtctggatcttccttaaggtcgcgtgcaagctcaccgcagcggataccctcgaatccgtggtattctgtcgtgccgtcctggaaagttttagcggcaaggcataggcgataattaacaccgatccggtttaccgcgttgatatcggttaacgtgatggattcaggcaaatcattagccattgcaggcgcgttaataccaacaaccagggcaactactgcgatcatctttttcatgctttctatcctcttcctattgcgtttaatagggcgttaccgcccctgcttaaagtttattgattgatgttataatgaaagttattttcttcaacagcttttaaaataaaagccgtgatcgctttcgtgtctacttgttcgaacgtatcagaatagcaccaataataatcatctcctactttcggcgggtaaacctggacaaggagatcgccacctggttgatctataagctcaacatgcaagccggatgattcagcggttttaacggcggttaatactgttgcaatactcataatcttttcctttctgatgtggggcgttgccgcccctttgttgattagatcgctgtttccagtagttcggctaacgtcttaccgcctgatagttggtctaacgtttgctcttctggcaagaaattagctgatattaatccgacttgattccctttgtgccaattagcttgacggatgattccatcctttaaggattttaccgccattgtgcaaccgtcgccaaatgattcatttttatgatccatcacttcataagcgcccagtttgctgctgttaggcaccattaacgcgaatacatcaccaacaaccagtttttctacggtaacaatagcgatagtttccattttactttcctcattatcggcggcaccattgccgccctacgtggtaaatattagcacactggcgatctgtgtcaatactttttattcaatataaatcaaattatcttcaccaatgtaagccgacacgttcggcataaacttagctgcattggttaggcggttggctgtgttagtggtgtatatttcagggcgatcccaaaagcctacgccgtgactgttacgggtaaaatgtaaatcattcccgatctggttggctgttgcgccgtcgctcattgcctcagaaatagcggcaagattagcattaataaaggctgttacatcctgggcgatccgttcgtccgcgctaggagaaaaatcataatcgtctaagttatcaataaaattaccttcatgatccctggctgattcagcaaaaataaaggcttctttataaccgttgatgatttcaataatgatttctgttttcatgctgtacgctcgacaataataatagtttgcgggatctcgttttctactactaacaaggcttttttctgtgttatagatattttaacacgttgtaaagtagcggtaaaatctttcatttcattatgcgcggatttagtgcaatgccagatctcaggcttgccgatctgcaagccgcgtaaaaattctgttttacctgtcttattcgtgatcatttgattcaccttatccaataccgtgggaagataggatcatgttagccatagttgcaaccactccaaccgataacaccaggcccactactacaaccgtgaaaattttctctaacatgattttaccctcattagtgggcgttgccttattgccccgccctattgacggcggggaatactcccccgcctcattaattattcttcatcgtcgccgttatagtattcaatgccgttttcatcggttacagtgatgttaacgccaccgatagcaacacgatcacgggcaaacgtcatagcttcatttaaatcttcagaccaaaaaatattgtgacctttgcgcgatacataaaacatagtcgggatcatgatttgtttctcttctgttgtgagcggtggaccattccgccgctctatgtaataaactttactacgttgtttgcctactgtcaaattattttttaatgttttgccctacaaaattgatcacttctttaagctgtctctctgtcatgatcgtacctggtaactcttctttaatttcttccggtaaatagatattaccgcgccaggttaacaaacaaccgttttgatcttcgcagatagaataagaaaacatccactcgcaatcattctcaagctcccaaaaacacaaaaagtcttctttccctaagttttcaacggttaagcataatccgcgacgtgctgcataattagttaggctttttgtaattttcatgatttttctctcttcttgtgagcggcaccattgccgcgctacaagatacatattatcagactacgcggacttgtcaaactattttttctttttctttgcttccaaccgtctaaggttacggcttgacttattgcgcaaggcgcttagatcgtcgtaccctttccatgcttgcatatctttcggattcatgttatccccttacttgttagccggatcatataccaggaccgcgccgcttgctttcacgctttccgcttcatagttgcgcagatcgttttctgtaagattgagcgcgatcacttcctcgacttcaacgccagcggcaagggcgatctctttgataatgtctgttattttcatggtgtgcaccttataagatgattgataagatagtgacaacgaaagcgattatactgatggcaaatgtcatctttttaccttcggatactaccggattctctaccgtgtattctactgtgtacttgctcatcgtgtaatcctctcgtttgctttgggaactattcccgccgccctatgaattaaatatacactatctcaaaatgattgcaagcctttttctaaattatttatgcagtaaacaaaccatcatttacaacggcggcgatgatctcccgtgccattgattcagggaaaggggcgctatagtcctggcttgtctcgtcggtatcagtgatccaatgataagctaacgagtaaaagcctttactaactgttttcagtgagatgatgatcactcggtcaagcttattggtgaatgttgccgttaatgtgttaccctcctgcgtcatgttggacaagtaagcgggatcattaatcagggaaaacattagcacggtgttagctttactcatgattgcatcctcaaaggggcgcggcaccattgccgccctacacgataactataacacaatgtaaaacaggatcaacctttatttttccacggtgtcagcttgttaatcattggtgataaataacgtttaccaacgtaaaacagcacgccaactgatgcaacggcggcgatctcatgcaaggaggaaggacgcgcggtgtcatagtaggtaccgataatagcaatagcggctactgcggcaatgtctgtaataattttttcttgttttgttgtgatagtggtcataatgtttttctcttttatgtggtcgggaactattcccgccctatgtgatacatcttatcctacctggttatggtgtcaagcgtttttaaagatattttatgctaatgacgttttcaggtttaaccttgttgtgatgtgcatcaacatagatcaaaccatcgtcagaaaagcccaggatcacaccctgcgcaataaaaccgtattcgtcacggtaacgcacggctatttttaatgttgcgttataggctgttttaagctcgtttaaagtcatttttgtttatcctctacatgtaaaaagaaagggcgcttggtgcgcccttgttatgttaattctctaaatccatccaattatatttgtcactcttcccgcgctttgttttattgcgcttgtcgcctttgcggatcacttcctcattccaggccttgcggatcttggaagattgaattttttctgcgcgattagatgtattcattttgtttcctctttatatgagcggcaccattgccgctatattattaatttaaactgttttgtaaaattgtcaaggtttatcattcataagagcaagcaaggatcacgcctgcaatataaagccaataattttccctttctcgttggattttatcatcgctgctattctctttaatgtatccccaggatgttaacaagcgatcaatttcccaatattcgaaagcgatattaatatctgatggcaaaccttgtaaccatgatgcaagtgctttaacaatactataattaaaacgtgattggttatcagggtaccagtactcggaattaaaacggcgcatcaattccgcgatcttctctttttcggttgtaataccataatcattaaaatcgatagcggcaaggatgtactctttaattgcattttgtgctttcatggtctttctctctttttagcgggaactattcccgccctacgtgtaacagtttactgaaaattaaatccgtgtcaagctttattttgttactaccatttggtaccctgtttcaatgttagtaaatactgttatcccatcctgacgttcactcattagcatacaaggcgataacatcctttctactgccttgcactcattgtaaacggcggttatatctcctgcggccttgtcttctgtagtaaatgtctcgtttacgtaaacctgaccagcaacaacggcgacaatagcaaaagtaatcatgatgtttccttgttaatagtggtttctcttctctatagcgcccattataaggcgctataacgaaaagtcaacgattattttttaaggcttaaagtacctaacttgtcagcatgacgcagaccagggaagatccattttcctgattcatcttttacaggggctttaagcgtagcgcccaagttggcggcgttaactttacgtgcaacgttgcgagcggcttcacgggtagcgaaagtgatcatgatattttcctcttctctttgttaggcggtggaccattccgccgccttgatgtaatacagtttactctatttttatttctttgcaactacttttatattttctttctttgcctcttccacctgatccgcccactgcttaccccacgtttgagcgttatagtggtattgacaaagagcaacgccacgttttaaaccagagatagactttttaccacattggcaagttttcattttgtgatcccttagttgataattgtcaaagtaaaatcataatcttttgacagtttatattgcaccttgttaccgtagtaatcgcccttacgtaaagatccgcgaatacctattacatccttaacgtcttttaaatttttcacgcctgtaatgtctttattgaaatactcgtcatctgaaaataaaacttgacttgcattttttaacagtgtgcggatattagccatcgtgataatctctcttctgttgtgagcggcaccattgccgccctacgtaatacagtttactctttttctttccggtgtcaacaatctttttcagattaattcatccaggcataacaggcgatcaatgttgagatcggttaggctgatggttggcacctggtaggattcgatgtactcgacgctcgcatattctccaaaattagcaatagcccacttattcaagatctcacatacatcattaatctttttgtttgttacaactgctacaacctgggaactgccaacggtgccggatacacgaacaatattgatagccataatctttaatctctctgttgttggcgggaaaccacttcccgccctatgtgatacatcttacactaattttaaacgttgtcaaattattttctatttcttccgtaatagcccaatttatgatctaagctaaaacgttttgtgagataggtcgaacgatgtttcatttttatgtctcctttacattccaaccatagcggcgcggattgcagttttactatacttaaagccgtctggcgttgtagtgattccatcctgtttcaaacggtagcacattaaatcagccgcctgatctttattaccgcactcattgatcaatcccaatgcaaaccgatacaatccctcatcgttattaatccacaggcttacattccaatgagttttgtttttgtgaccgttgtacattttagatttagccatgatttttctctctttttcagtgaggcgggaagtgttcccgctctctatgtaatacagtttactctttttattcgtgctgtcaatgctttttatagatcttttttacgtttaacatcgcttgcatgactgtatacagtgatcccattgtcacggcaaaaacgggaaagatacgcgccgcgtgaaaattccttgccctcaaggttgataaccttgctaaattcttccagggcgacggtatcaaaatgatcaccatagccatattgaaacggaacgttaaccacctcgatcagtttaccgccgatctcaacgtcaaccattaaagaaaaatatgtgttaccgtaggactgttgaaaccagcgacgggcgctaatatgcaccgcctcaatgctggataattcaattttagccgtgttagccagggtagcataagaggaaacattaatattaatagccatgatgataacctttttcattgttggcgggaaaccacttcccgccctatgtggtaaactataccggaattttaaacagtgtcaaccgtttttattcgtaaattttaccgctttcggcccataccataacgcggcgcatactatcccaagtgttctgatccaagtttaagccgtgcaactcttcggtacttcccagaccaggtaccgccttgttgtaagtagcatataagcgcccttgatactcatacgccagagtaagatccgtttgaatcatctttttaagcatagcattaagcatcatattagcggcatgttgatcacgttttagcggtaacatatcaataagggcgcgataagctgaaacatgaatatcaaccgtttcacgtggtaaatttttcatatcatcgcggaaagataccagattaaacatgatagtattgatttgcttgctcattgtgttaccctttttgttttggcggggaagtattccgccgccctatgtgatacatcttatcagattaaaaaacgttgtcaagaatatttttagaaaataatagccgctactacaacccaaaatggaacggcgaaaatgatagcgttaatcgttccaaccgtcaagctattttctttattaatcattgtaaacctcttgcataatttcactatgagcggcaccattgccgccctacgtggtaaatattataccagtggcggcgatatgtcaaagactattttcaaagttttctagtgcgatccttttttcttcctctgtcggctgataatcaggaaaagcatcaatacacaacaacggcgggatctcttcgtactcttcctcttccgttgtctcctcgtcttcctcctgggctatggctgcaattttggctaattttctttcggcttgaatcaatttaccatcaagtacgttttttgccatgttgtaagcggtccaggttatgcgcttactttctaatgcttcatcaagcgccgctatcttgctggggtattcatccaggacacgaaaagccggatcaactttgtacaaaggattatctttcgcgccgtcgctcgcatcttccgcactggataaatcagcgttaccatttgccagccagccagggcgcacataacgcgcttttacgttgccgttactacgtgctaatctgttacctatttgctcaccgctatatcccgtggattcatcgataagatgatcgggaatgatgcgcggatcatctgcttcatgttttacccaagactggatcacatattcgcggcccatattaaccaccttttttcaacaattggcatactttaaaaagacttgctgaatccttgcaagggtagaaagatgtacgataatcacgtacgccaaccttgcggatctcgatgttctcgcatgttgtttgatcaaagatatttatttcatcacgcgcccttatataaacatagtggggatgtattaacgcatattttgctaatctgtaaaaagcgttatctttgaacgcgatcacgtgaccttttaatttgatgtaaaacattctaccaccttacaaatagcgggaactattcccgctttatgtaacatattaacaaattattcgttaatgtcaagccttaaagcaaaaacgctacatcttgcgcttttagtgtagtatcaaccatttttgggcttttataatcaacaaaacgcgcggttttttcaatagcgaaaccgagataatcttgtttattgaatttacattctttcttaaatgtttcccgcgccacggtgatcgctgcgtccagggataaacgcccgtcaatgttgatatttgcgaaagatccgccaccgctcataggtttaccagtggtgccagctacgatcattgttgcgctatatgatgccatgattataatctctcttctgttgtgagcggctggaccattccgccgccctatgtgatacatcttatcaggttgtggcttgttgtcaagacttttattctattcctacaatctgcttaacgcgcttgcattttgcagtatgctgatcttctgtaattactcccatttctaacagtgaatcaatgaaagataaaaccgtttcatgatcatcctggggatgactgaactcagtaaaaccgtttactattacgttgatttgtccagctttcgccacaatgtgcaatgttgccatgatcttaactccaaataatgagcggcaccattgccgccctacgtggtaaatattatgccgccacctggcgaacgtcaatcactttttcaatcttttttattttctttttgcttgctatatataaaagaaagggctatcatctttctaccgcgccaaccagggcgcacaacaaagaaagggcgacaccatgacaaatactacattacaccttatgcactcattgggctggcaaggcggcacggtgcaccaggtagcaagcgcaacgggcttaaaggttgaaaccgttctcaacctgggcgatcataaggtggaagagatagaccattttgatccgtttgctgatggttatttatgggcttgcagtggcggcggggaaagcaagatcccaccaggggcgcggggcgatatgcatttttggttaggtgtcctttcctggcaagaaaacaaaaaataatcttgacaagcccagggatcgggcttatactgttttacatacagaagaggaagcgccaccccacggcgtaaaggcggggctaacataccagggcgctggttggaaggttgatcgccggtaggctgggcgctatatatttgtaaagaaatgtaaagataaaataatcgcttgctttcctggctggatctgttattcttattacatggcgaggggacacgggagcgcgagacaaacaccgcccctgcgcgtcattctctaaatgagaatgattatcatttaccttatcaaataagaatcaatctcatttataaacctaaatgcgaatcattatcatttgcatttccatatgagaactcttctcattcccatctgcataaatttttgtaaactttaaattttgcttttgtactttcatttttgtaaaagcaaaaaaactttaaatactgtgggcgcgcaaaatttttaaaattttccttgacttttgagttttgttttgtatctaacattttaagataattttcctaaactttaaattttggttttcaactttcacttttgtaaattcttaaaaagtttaaataatttgagggatttttgagataataggtagtgggaatttgagataacctttaatcttattggtaaaccttcaggaacccacttagatcgaatcctagtgatgcaaactgaaaaagataagggtttgtataggacaaaataacaggctctctacggtgctgtaggagccttatttctgtgctacaaagtgacacctttgttctaaaaatgaaaaaagttaaaatatcacaaatatcgtctttgcggtattttgtctgggaaaagtatttcgatcagtttgttatgatatttgcctaaaagattctgccctttccaggtggcagggtttagcacccctggttcatacatccccattccaattccgtataccttatcgtaaggacttgcttcaacaaatgttttgtattgatatagacgtaaatcggcatacaagtctggattctgtacatacttcaagcataaacctcgcataacaatacgctcacgattctcttcccatattttgttatcgaaacctgctactgccctacccatacgtttgtaataagcttgtatcttctcatcttctcgttttaagctatgagtcagaatagcttgtgcggtgttgtgatcgttaaagagcaaagctttacgccacatcatgtattgttcgacacaattaaatgtctgattgtaacctggttcggtttctgtaaagggtgcaatataccaattactaaaaacatctttatgtgaaaagaagaatacataatcatcagttattcgcatttttcatactctccaaaatttcacgttgcccttgctgataggctttcatcagaactactttaaggtttgtctccaagaggtcttcattgtaattataccgctccacaacatatgtcaattcatcataaagatcaagaacggggtagaatacagggttatctgctaaacgtgccatagtttctcctttgttaatccatccaccagtagccataaactgcgtctgcgtcgatgagtttgtgtttgttgttccgcatgtagtctaagataagtagacgtttattgtatctggagagattatgaccctcttttccgccatagtcaaaatattttgcaagatctttacactcaaacaaaatcagttcgggagtttcttcacactggtctgtctggtacacttgacgaatgatcatgtatacatcgtcgtcttccgctaaatgctctactggtccacatccagcaggaacaaattcatagtcccatctgatcagatgtttctctactgcttcaaagaagtccattctttcgttgcgcataatttgaacgtcaactaacatgtttcctcctgtaatgttccaccataaagtttataaatgttttgtattgaggccatgcaagataactctactgtctctgtgtagccatctggcatagcatatttgctcactgcttccgtgtgaaggacacgcttacaacgcttctctgcatctttgcattgcttcacagtagggaagtgccacacttggacctgtttcatagtaaacacattatgactattttgttttttgatacggcgatcactattattggcaattccaaatttcaaagcacagagaataccttgttcatcatggatgtggttaatgtaacattgttgttgtgtctggttagcacattgcggacaaccagtaccatgatttaacaaaccactggcacgtcctatttgtaccccatgtttcttacatccatatttaaacttacctaatcttacagacggtttcccgatccaacctacccagatataccctttcctttctactgccttttttaccctataggtaatctgtgcttctgtgtaatgacagttagttgcacaacggcaaggaatcttcccttgtttaaggtcattccctgcgacataaaacttaccatcacacaccccagcttgcacatactcatcgtgagagcaggcaggacactctacacaccataaagaatcgcggcctcgttttttatctgttttagtgaagattgtaccctctttaaactttcctgtattaatgaagtcttgtatgtgctgatcgtctgggagtattttagaattcctagatcttttgttaccgcatttaggacaaccactaccacccttaagattatgaaatgttaatgttcgccattccccgtgctcaggacaaaggttgatcatctttgtatgtacacctataaactctccattaatgcctaaaatcttgattcccttagcttcagcaataggcttacattccgaaaggtaataatctaggtttttactgttgtcttgtcttgccttctcatttttgcacccagggcaaccagacttaccacgtttaaaattggcaaatgtggatgttctccactccccatgattagggcagaagttgatcatcttcgtgtgcacacctttaaatggctctacaatccctataatctcatatcctttagcttccgctattggtttacattctcctatatactgttctaacgtcttcgccatctttgtctatcctctctccctataagtctgaaatcaatataacaaaacccgccgaagcgggtttagctatttgtgctgtatcagaacacgctacccagtgtagtggaacgttgtgttacaggctccagcaccactcgcaaagtgtttcctggtgtagtgctcatcacaagaatcttaccaccttcaatcagatcacaagttagaccatccgccgttgcacatagatcaatatgtggatcaccatcttcatctaggataacataactgttggtttccattgcttccacttcatcatctttcaagctttgtagatagatgtttagggaacaagcacccgacaaaaacttagtaggggtgaagtcgtgccagttattgatccaattcaccttcagcgttttgccattagctacgatagaagccacattccatttagcatcgttgcctaaagaaatacccatcatttcaagacattctttaaaagtcatttttggatagaaattcaattcttctacaatgccttgaataacatcgaaagacaactctgtgctaacatcccataaacgttggaagttatctacgtgctttttatttaccaaattcttatccaagtaatctaacatagtttcactagacagtttatcgtaattgaaactgtaataaatacggctaggacggtttacaaaatattccgagatatagccactatttttagtgaggatgtaaagtttattcccacttcctgttccatccaacaaggtcaacagtgcttcctgatcttctttcttagcgtacactttatcaaattcgtcaaataaaacaacagcacgatctgtaattttactcataaattctaagaatccagcatctgtgtaaggctgtgtaaccaaaatggttggaatacctaaaccttctgtggacattagggacatgctaattagtttagctaacatcgtcttaccactgccttttgtgcctgtcagtaacaccccagtatttttaccttctttactacgacgagcaaatgttttaataacatgtgcggcgcggtctttagtttccccataaagagtgttcggaagagagaaactttcgccacgcactaaagagtagttaccgtgcggatcacagttgacaacccaaactccagcaggaagtttattagcttcaacaccatattccttatttacaacagctacatctccaaaaatagtgtatttcattagaacgtttctccttttaaaataattactgtaaactttgcagttttttcactcttaatgttcttgactaataagtattctcctccaggggcatctttgtattgtttaatagctactatttcatttttgcaagtggctataatcgaataaaaaggacgttccgtgttatcttcttttcctgtatatgcctcaacatcttggcactcttctagaacaccattcccaggtgtatagtaaaatttgttgcctgtgatcacaacatctttaccaatatccgttttaactgctacaaatttttgagcgtaagtggtgaagcttacggtagccagcaatgtggctaagattaattttttcataaacttccctcctattcccacaacatacaaatcgcagcgacaaacatattagcaatagccgcaccaagaactataaacatcgccagttcagaacaatattcaccaaccagaggggatactattgcacagagtagtcccaatgtcaaacacaagttgatgatgaagtttaaaatcatattctctccttacaaccaaccctttgcctggtagcctttagaaaatccatcttcccattcttcgaattcctttttgtgtgtcattggacagtatggatttactttattaaacataccaaacttgaaccctgcggtataccctttcttaaaagctattgattctaaagagatcatattctttctcctcttagaactcgacaatgattttgtctaagtgttctgcttcaacttgggataatgttaacacctgcaccaaacctctgtcaataccaattttaatgacttgcaaataatcaccaaaaagatctgataaagaaatagcatgttccgtaactacaataacttcattacctttagtgtattctttccacacccgaccacatttataaatgtactctttttgatcttcagtaatcattttaggtttgatgcttaacattacatactcccgtattctggttcaatattatgtttacgacaaatggtatcccaacgttcattaccaaaacttcttccatgtagagcgttaaatgcgaaatgctctgccaaccacaatactacatcatcaggatctgtgtcaagaggaattctaaactctcgatcataacgcatacgcagaataaaatccttatcatcttcaaggagatacatgtgacttttagcccgtgatgccaaatcaaagaaaggaggctcatctttccaaacgtgacgctcaacttcaataccatgatcaactaccagagtagcgattacctctctcatttctttactaaacatattacctccactaaccaattagttcaaccgatttgcaaatgtaaatccaaactggagtgagcataagagctataccgaatgcacaattccacttaattcccatttcctctattagagacatgattaagccaacagcactaataatcagatagatccctaagaaagccattgtgaagaattcagacatatttagctccaaagattatcaaaataactagcgaataaaagacggccctctaaccgtgccttcctttgatcttcagcatacacgatttctggattaccactatcaataaaagcattctccatcttttctaaaatatttaaccactctcttctacctgtctcctcttcttcatctgtatacggcgcacttctatttggatacaaaacacgcatcagtttagtggggatcatgacaaacgtgctattgtttgccttctcacggaaggctttaattcctgcatagattaccttccccaaagctgcatccaaatccatcaggtcactttcttctatttgaatttcatcgtgtacaattttcatcgtgccttctcccgatctgcatctaatttcaacaagaaagctgtatactcttccgaaccttggtgtaaaaacatgtgataagagcttaccagtttcttcgttgtcgtataacgttccatttccccaacaacacctccacctagtgtgtagagcacaataccttcaactccaacatgggttactacttcaaccaaatctgtgttatcccaatagtagttaggtgacacaataagatcgcccactgccagtttaatgtttgctacttcgtcttgtgaataatacatttggttctcctctcttaattgatggaaatagtataccttaccacataatctcacgcaactctttttcttctttaaaattacgtaagttttccagtgcacgatttaggtcttcttccgtctctgctttctggactttttccatcattttcgcaacaatatttttcttaacttgcaaatctggaacaggcatacgtttttccatttcataagcttctcgtttatgacgttcctccatctctgctttttctttcacccaatccgcctcgtccattgcacctttgcagacacaagtgtagcctttaatagtataatcaccatgttcaataccaggttgaagagctaaggcttcattaccacaatacgcgcacaacggacgacctggcattttcaaagttttggtagccttagcgccacattgactatcccagcttgttttataactggaaccgttgtctccgtgagaacgaccaaccaagaagttacggaagaaacccatagcacgttcgtcaatgtaatcagggtaagccattatattctcctttcacattaagacacgttttgtgtcgataggtgcattatcgaacatgttaagaacattgtcaagcagttttactaattcgtttgtaatttgttcctttgaacgggaggaagtgagcactttgttttcaccaaggatatgcataacttttataggagcgtctaaaatgatattgtgttgacgatcctcttcaaaagaagccaaccatttctcgttgttaccgtgcaaggctacaataacaccaaaaccgttaattgtttcagaaatattcttagttgctaaagtaatgttcatcatctacctctcttgttaatgttcatacatgatacctgaaaataatttcagatcaagtattgacatactttttaatcttgttaagatcaactcctaccaaacaaaaggagaaaacaaatgcaaaacatccaaaacatcaaactgacttggctcgaagctaaagaagcaatgaaggaaggtagacgtgttgagcaccgccatttttgtggtggcgaatggtttgagatgcgagatggtgttattctcgacgaacaaggttataacatggaaacttggttcagaggggaagcttggcaggaagagggttggtctgtaattgaaaaagaagatagtaaggaagaaggcgtgttgtctaatgtagaacgcgagatgagggaaacaaagatacctgctttggattttgtatacctggaagaacttgtggcagcatcttttgtagcaggaaacctttggaatacacctgttagccgactggagaaccaaaataacccgtctgctcgtaatcacaagctttataagggtaaaggtcacaacaaattaaagaaaggaaagaagaaatgatctacaaaaacaagttcactggctgtctccgtaaatttatcgataagaaagggtatgtttctcttgacaacggggctacgtggatagagaatggtacttcagaacaattgattaaagctttgccttactacgaggaggtttaaatgtcaaacagcctgtttagcgtgtcaattgcaccacgtgtcaacatccgttgtcagtttgttactttacctggtgtaacgcatatcacattagaatacgatcctcgttcacgagagaacccaattacaggtaagattgattcagcctacggcgacttcctgatggatcaccaagcgattttatgttgtgtgcaacgcttacaggcagggtctttatacatctttaagaagtctgtgttccaagagattgctcaagcagttgaaagcggtatggagactcttaacgctatgattgaagcagccaacaagaaggaggttttatgacactttatcacgtaactacagaaggcgactgtgaaggccgcactacaaaagatttggggtatgttgaagcagattcacctgaacatgctatcaaatacctgcattccatcgggaagtatgcgggttatgagtatactatctacgaggttgaaattattaaccttatacaagtggcaactcctatggcagagttgaaacatcttaaggcaaaagtaagacctggtatagtgtccgattacaaaggggaagtaaagtgagtggatggattaaagtatctggtaccgaggatgttcctatcggagagtggcttgtaatcatggcaaacggcaagaagggattctgtgaagttaacaaaacacgcaatggcacgatagctgttatcaatggtcatttttattttgacttggcacctgtgatcgcataccatgcactaccaacatatgaagaggagattaacaatggctaacaaagaaccacatgtatatactgcactggtaaagcacttgaacaaagaacgtaacaggatcattgcagattatcgtaaatttaaacgtagtagtctaaatcgtaaatacatgccacatgcagcttttgacgctacacgtgcagtatttcgtgcaaagattgcggaaatggataatctgcttaagcttgcacgattatctattcatgcatccaaagtaacaggagtcactcacttctttgacttcgatactaaagcactatatgcaccagaagcaggtgaacaattgatttatctacttaccaaaaaataagtgttgacttttttcaaaaaatatgctaccctaaccccgaagggtcgagcgatagcgagtatagattatgtaatagattaatctctatcgttagatgttcgaagaacagagatttaagtacttgttcttatcggtagatgatcgtaagatcaagaacaataataggagagtgtgttatgttagtcagtgaaatactacctgactctaatggtaatcagatcactgttgattggtttcctggtactggtacactagatctaactgctgaatggtctggtgatatttatggagaaggtgtattatcattgtctacagaacaaatgatcatccttcgtgacttcttaattagtttagatctaggagaaaacaaatgagtaaaacttctgttgagtttatcgagaaatcccctgttccaaagttataccgtttatctgaaattgaacctatgtctgttttctactactacgattcagaagtaagtgaacaagaacgggactactacttagctacagatacaggtgcagtgtgcttaacaggttctcgtgcaggagaatacattgtgactagtcgttttaatgaagcagattactgttgggctaaggttgaaactaagattgtcatcaacatcaccaatcattaagactaatctgacaaagagttacttgacatgtgcaaagctctttgtgtagaataaagattcacttactaaggagattttataaatggctacaaagaaacagaaaatgaaatgtcttactctcactgaactgggtcaagcttccaactgcgttactgttggtaagatttattctacagttcttgataaagacaacaacagttttactttcaaagatgataacggaaaccctcaagttggaatctacccttcctgtgcttttggtacttgggaaaaagtttaattaactgatcggagggagtatgcaagacaagaaaaacaatgttcgttatgctttgtacggacgtatgggcgtagactgtgaatggagacttctgttacagcgtattccctcttatcaagaagctatgacgaagcgagaacattataagaagacgtggagacacgttgtttgcaaacattacgaggaggcataatgagcacattatcaacagcagtcaagttccaccaggattatgaagaagctgtcaaagatgctgacaatccttcctcattagtgtttgaggatcgtacaggtttgaagtgggttaaatctttcgatgagatctatttctggtatccgcagggagccacaagcgatagttctctgtatagctacgaactggaataccgtggccctgtagacgaagatgaaaactttatcatgtatttcgtaaataatggttgcggtgaaacgtttaatgttgtattctctaaagcagatgagtctgacgaggaggtgtaatgttgtataatccgactttaagtttggcagttgcagcccacatgctgtacaatgattctgtgaaggatgagttgtcgcctgtgagtttagctttctatctgatagacaaggattggatgttctctggtgaggatgaaatctactacgtaaattcacgcggttttattgaaagttttgaacttgaacatcgcgcaccaacagcagaagatgatcgttttattatgttctgtgtaaatgatggttacggagaggtatatgacgtagtattcttcaaaaaccgacagaagactgaagaggaattctaaatgaaaaggctactttgtgtcctgatgttgatgattgccaccaatgctacagccgcccgtgatggtttcaagattggggtggaaggtagccaattcttcacttggtatggtggtgaagctgtctaccaggtggatgataaaaagtataactgtaagtttgatcgtatagatcgtggagtggataattggggtgacgcttacaaagccacaggtttctattgcacaaaagatttatatattacagtcaaagaaccacaagacaacagaaaggtgttgttcattgtgctaaaccctgctaaatttgaagaacgtaaagcttactatgcagatatgttcattgaatacaaaaaggaggaaaaatgagagaaacaatcgtagatgttgttattaatggtcaacgttatgtgcctgttgtagagaaagctgaaccagatgctttagatgttgtttttgaagacgatgatcttggcactgttactgtgcgtgaatatttcagacgactgctctctacgctttgggaagaagaagatagtttcagtggaaaacgccctttcggtaactcttgctggcagttcgatttgattattggtttgatcgatgctgggtacctggaaggtaaagtttatcgtgacgaagatggtggtatagaagataccgactatgatcgcgaagaagcagataaactcatccagggtctgatcgcacgtatgtgtttaggttaatatgaaggtattaaacttttaccatttaggcaaaacaatcccagagggtgctgtttatattggtagagcaatgccacatatgggactaaaagcctctaagtttgcaaatccttttaagttaaaagatgatgaaccacgaggatcaaccatacaaaggtataaagagtggttgtgggagcaaattcgttgtggtaagatcactcttgaagatttattagagttagaagggaaagatttagtttgcttctgctcacctcaaccttgtcatggagatgtattggtagcagccgtagcttgggctagaaaagagtgggacaaaaggcatcctacttatgtagacaattattggatttgggaggagaatttttgaaaaatattattgatgtagatgtgtttggtatcacaacactacaagacaaacaagcaacattgtattatcataaaaaagaggaccaagcttacatctacatcggggcagaaaagtttggtgtcggagatatggttttaaatgttgaaggagatcttcctttagaacaaaagttgaaagtttttcgtttcacaggagatgttaaactagacggcgttatcgcaatcctacaagaagaccattattatatggtaactttcgttactcctgctttctctggccttaatggagaagaagctcttaatgatgttattatcttaaagattggtaatgctctaaacgatgtaaactaaagggggagtgagtatgttctttgatgcaaattctctttttgtatctacagcagggcagtctgctgttaatgcagaatttgaggaaagagaagaatggattgataatcacaactacgatccaagtaatttaaactggtgtagtttggatgttggtaatcttagtcgagataatgcacttagagcatactactctgtatatactgttgacaatctttatcaatactacgtgtttcgtagtcttattgttaatcttaatttagctggacgtttaaagaacaataaggcattcacattgggacattatattcccttccgtaaaggtggtgtgcattttccatgtaattggattattcaaacacgaaaagaaaacagtttagcaggagacaccatgccaagatttaaagataagtggacatgggaaaaacaaatggggtacattaatgcacatttgccacctaatctaaaagaaccttatgccagcagagctaaggaaatgatgaggattgtcaagagtttttattaagagaggagaaatacgtgtctacacaattgagttttacacctaccgaggaacaatacgctattgaaagcgggtataaagaaaatcgtttcgttacaattcaagctaaatctggatcaggtaaaacgtcaacactatatatgttggctaatggcacaaatgatagtattctatacttggcgtttaacaaaagtatggcagaggaagctcggcgtaaaatgccgcctaacgttatgtgccgaacgctacattcaatctgttaccagcgtcttccacaaaaccttcgacacaaattatctcgtccagaaggtcaatatgttaacgtagctggaactggtagtgagattgccaaaaaattcaagattcagaacttgattgataagaatggtaaagtgctactgactcgtgcaatgatcgggttaatggtaaaacaaactttagctaaatatgagttttctgacgatgaacatattggacataaacattttcctaaagtgcatatgtcagatcttaaacgtaagaacatcaacgaacaaaaacttattaacgaagttgttaaatatgcgaagcaactttggcgtgagcgtatcgatcctaaatcagaaaccattatgactcatgacacctatgtgaaactctttgaattgagcaaagaagacttaggatacgatattatttttggagatgaattccaggatgttaaccctgcattcctgtctatccttcgcaatgctaaatcggctaaacgtattgttgttgtgggtgatgaatttcagagtatttatcagttccgtggaagccagaacatgatgaagcaaacggctacatatggggttgagttgcctctgacagcttgttttcgctttggtcctaaagttgcggacattgctaatgcgatcctttctgatcgttcacctctgaagcatccgctggtaggcaaaggttatgatactgtcactggaagttttcattctgatgtggtagattatagtaaaccttacaccattattttccgtaagaatttgactctgttgctcgaagctatggaccgaatcgctaacggagaagagattattattaacgtagataccaaagatttcatttctatggttgatagtgttaatgctttacgtagaggcgatttggacaaagttaagcatgagacagttctcccttacacctcttgggaagaatttgtagagtgtgcagaatcagatcctgatgcaaaacgacttttaaacattgttgtttctggtaaagctaataatattgcatacactttacgtatgcaccgtaactctccaacggctaaagttactttaactacagcccacaaatcaaaaggtttggagtgggatcaggttattgttgcaaatgacttcccttccaattatgataagaagggtgagtgggtaggtcttgaagaatcagaacgaaatcttctgtatgttgcacatacacgttgtattaaatgtctgcaatggaaccggactgttcaagagattttagacatgcatcgtttaggtaagagcacggctcctgtatccgaagaagataagcttatagctgcccttgctcgtaagattaatcaaatcagagaggatcgttaaatggcggggtatcacataacagacattaagaaacaaccttatggcagcatagaaaagattaaagaggaagtagaagaactttttgatgctgttgagcaggattgcaaaataatgcaacttgttgaattgtcagatattataggtgctgtcgagggagttttagaacaacagttcccagatatgactttacaagatttgatcattatgtcaaacattactaaacgtgcgtttcgcacaggaggaaggtcatgaacacaggtgtgattgccgaagccatccagagttatggacgctttgctgaaatgttagatgttccttatttagtggatgcagatattttggcattcacactgccacctggcaaagaagatgctcctttggggcatcctaatgggaagttttatgttgccagtgccgagcaatcattcttgcaaatggaaaaggatggctacgagtttatcgctggtcatccctacatggccctgacaccatgttaccgcacagaggaagtgttggatgattctcacttcaacatcttcttgaagctggagttgattgagttctatcccagctttggtgaagtgaaagagtatgccgaagaaatgcgatacttcttctggacctactacaaagtccaaacaagaattgtagaaacagaaatagggcttgacgtggaagacatgaatggtttagaattagggtcattcggaacaagattgtccccacaaggtcatccatatatttatgcaacagccatagccgaacctcgtgcatctatggcaattaacaggaggaatcatgattaataaatttgcccgtaaacaggaagttgctgaagcagcacgtcgcgctggtgtgtatgacaaattgtgtgaaggtattgtgaaggtgtatggctgggagggagaagatgatccaacctgttcaggtgattttgcaatgaacgatatgaccttcccatatatttcctacaatgcctccactggtgaaataaacggtgtaaccagcaccagtgttgatcgttatccagacgcagaacaaaccacaggaggttacatgttagatctgattcttaagaaatcagggttattgggaatcgaagacgttttcgtagatgcagatagtttatcattaggagatgttgttaaactactggtagactttagatttgcaacaggtaaaagcttctggaagccagaagatgctgaacgtttgaagacaatcgacccaagtatgtatgaactgccacaatataaagataaacaattccgcaaggttatcttctttgatcgtcaagcaggtttagatgcaatgcatattgaagttggggatttaccacgcccacactctgctgctactggtgatcgcggccttgtagaagtcacggcccaagaatttaaaaatatgtttgctgcgaaatacgatgaagctgcgcctgtgccaagtattgaggagcaagaggaagaacagtttgtagcggaagatactgcatttgaatatctgccgaacacaggtgttatgccttcagaagatttcgtgcaggttcgcttcaacaatggtcaaacagatgttgctaagggaacagtattcacttggaatatcgacaatctggaagagtgggcgattaaagaatacttcattctcccgcgcacggcaaaatacaactataataatggtggacaccgcccttgtggtggtcacgtagaagttctctttcgtgacggaactacggatagtggatcggctacgcatttcacttggggtcatgttggatatgatcgggatattgttggttggcttccaactaacaaccctaaaccacgttaccgtaagcctcgtaagaagccgccagtaggtaagaagagtgttgttcgtgtcctttacactacacagcagacatacacatttaaaaatgtggtagacgtgcaagtgaccgaggagcgagtttatatccaatatgatcgtgaagttacagagggtattaaagaatcggttactaccgaaattgaaaataagcttgttatggcaatcattgtggaagaaagtaaagtagagaacggtaaagcctacactgttcgtgatagcttcttgcgtaacattgatggaacttgggattataatggacacgatgggaccgctgtaaagaacggtaagatcatgggaagacaattcctctccaaacgataaaacttaaaggcgcactttgcgccttttttgattttcctcaaagcttttttcggttggaagaggtagaataatagttctactcacaaaataaggattaataatgaaagatttgtaccaagatgttttttcttttttagatcgcacttcagaaacatacactagagagaatgcaaacaaaccttcccacctcgttaacacacacagggatctcttggcgggaattttaagcaaacatatggcaatgacacagatactgccaaaaaacttgtcagaatggcatacagaaggttttggacatattcacgacttggattatctgctctctcctcttacaaactgttgtcttgttaattatcaagatatgttagaaaatggttttaagatcggtgctgcacagatagaaaaaccaaaatctattggggtagctaccacagtgctaactcaaatcatccaagctgttgcctccagtcagtatggaggtcagacgtgtgcgcacattgataaaggtcttagtccttatgttgaagctagtttccttaaaaacatggagataaccaaacgtcgctacggttgttatgcaagggaagaagccctagaaatgaccgaacaagatgtatacgatgctatgcaaactctcttgtatcaggttaatactttaatgagtgttaatggacaatctccttttattacaatcagtatgggtctggatacctctgtttttggcagaatgatcacagctaactatcttaaagtgcatagtaacggtctaggcaaagatcatgtaacacctgtattccctaaagttattttctttttacaagaaggtgttaacatgaaacctagtgatcctaactatgaccttaagcaacaagctttggcatgttgtgctgatcgtatttatccagattttatttctgttcctttgaacaaagaagttacgggatcagcagatggcactgttacgagtatgggttgtcgttcctttttgtcgtactatgccaccgaacaaggagaaaaatacgatggacgattcaaccttggagtagtttctatcaacttacctctgatcgctgcggaagcgagaagtaaaggagaatctttcaccgacctccttaataaacatatggagatagcctacgatgctcacatgttacgtgtaaaccgtatcagacataccaaagcatctgaaaaccctattcttttcatggaaggggctttagcgcgtttaaatgccaatgatgaaattggtgttttgtttgaaggtggttatgcttctattagtattggttatgtaggtcttgccgaagctttggaaatcctttatccagaagaaaaagataaaaaggaacttggtaaaagcattctgcaacatatgaaagatgtgtgtgctgtgtttaaagaacgatctaagctggcgtttagtctctatggaactccagcagaatctttgtgctataagtttgcaacagctttaaatgaaaaatacccaggtttggtagaaagagactatcttactaactcttttcatcaacctgtatggttggtaacaaatccttttgaaaaatgggattatgaagcaggttttgcacagattagtaatggtggtaacataagttatgtcgaaacaccaaacttaaagaataacttagaagctttggagactttagtagactacgcttatgaacatattcactattttggtattaatcagcccgtggatcaatgctttaagtgtggttttcatggagaatttaaggcaacggctaaagggtttgagtgtcctgattgtggcaaccatgaagaagaaactatttctgtagttagacgtgtatcagggtatctttctgcccctaattcacggccttacaacaaaggtaaaatgcaggaagttttgcaaagggagaaacatctttgaattttgcaggatatgaacctgtagatcttattaatggtgagggagttaggtgctccctctgggttagtggttgtagccacggttgtcgtggctgttttaaccaaaaagcctggtcttattcttatgggaaaccgtttactcacgaaaatgtagagcaagttttaaaagatttgtcaagaccgtttgtaaaaggtttaacaattttaggaggagagccattagatccgcagaatatttacaatgttaaaaaccttatagaaaaggttagagacgtttatgggtcaagcaaagatataatggtgtatacaggatatacttttgaggaagtgcctttgtatattaaaaatcttgttgacattattgtagacggaaagtatgaaataaacaatccaactacaaaacctttcagagggtcagataatcaaagattatggaccaaaacggcaagtggttggcaatcagattaacttaggaggtttcatggttacatattttgctgtaggagatagagtagggacggcaggagaacttttgaagaacagtgatcgggacgttaaggacattcttgcaacccttgtggatattttacaaagaaaagagttgctttctgatcgagatgttgctaaattacttgatatcggaggagatgaacttcaaagacttccattttaagtgaggagataagtaatgcaacatagtgtagtagtcaacatgccgaatagtggggataaatcttttggtgtccctgctaaaaaagtgtggaaagcccattttcatcttactttatctaacgttagttctgaagagttgatgaaagcagctaagattattggtgccaaatgcacaacaattgatttacatcgggatacaaagtctcaacgtgatcgtatgttaacaaaatatcaaagcgatctcgacactactttaatgttgcagtgtgtagaaaaacttgagaatgcaggtttcaatgttgttcgttacaaactggaacgaatgtttaaacatgttgcctgtgttcttttcatggatctaagtaaagaaaattacggagaagttcacattaagacttcagcaaatgatcccgaagtagaaacaaacttcttccgtatttcttctaatgctgaagaagttgattaccgattttataatgctcgcttatattccgcagaagataagttgcgtttcaatgaagcgtatgatatgctcttgaacagtgggatacagataaaaggctaccacatagaacaagtggtagtggatagtaatcttaacttagacgcatggtgggcttaatatgactattaaacatatctcttttgatgtatggaacacactgatcacagcaaactccaattacgcagacatgcgagcctggactatcgcaaaatatggagatattacggaagtagaagcgagagcagcttacaataagatcaagaaggaactggacaatgctgcacttcagggaacagccgagtgtgcagcatatgcctgggaaagacttggttacgaacttgggataacacctttgaagtctgcaaagcttcgtttcgagtgtgaacaatggttccttaagtttccacctcatgtagctgtggatctaccgatgtacttgctgtatctttcggggcgttatgatttgagtattaagagcaataccaacttcatatcaggggatatcctgcgtcgtgcttgtaagtttgatagttggaattgttttgagttcacccatttctcggatgaagctttactttgtaaaccagatccgcgattctttgctcgcacgctcacgtattctccgatccctctgttagggcacgaagtgttgcacgtaggcgatagtgaagaatatgatggaaaatgtgttgacttaggatttaacttctgtcatattaagaaccctaacgacttactagagaaacttaaaagaggagaaattgtaaatgcgtaatcaaactcaaatttttgcaaataaatttgatgacctggaaactgctacttttgatatccaagattattcaaaattgaaatttggtagtaaccaagctgcgcgtcgtatgggtaaagaattggcacttcagttttttgctcaagtggcaatgaaacatattggggaacaatttgttatcattccttctccttataaccatgtgcataatgctgcaacaatgatgacagatcattttattaattattttaatatgttgcaatcacaatatgatcaaggtcgttcagcagaactggacattattcatcgtaaagttagctatatcaacgattatggtttcttggataaagctagtcgtaagaaacttattgatggggatacattctactgcaacaaagaattctggggtgataagaagcttgtattcattgatgatgtaaatatcactggaacgcatgaagacaaactgattgagatccttgatgaaaatcaagttaaaaatgatgtttacttcttgtattttgcaaaatacggaggtaatcaagcagataccgaagctaaactgaattttaactacattaatacaccagaagcgtttgttaaaatgattaatgaagaacgtgatgcacagtgtcttgttcgtcctattaagtatcttattggccaaaatcatgaagacttcaaaatcattcttagccaattgccacattactatgtagagcaacttttctttggatgcttgggagaaggttacaaccgaatttcaaagtatgctgaaaactttgaattcttggatcgttacacaaaacaacacattctgtaataaggagctaaaaatgcttaaaatgaaaacaggtaatcttattgatgccgctaaagcaggtgaagtaactgttattgctcattgttgcaactgtatgtgtaatatgggggcaggtattgccccacaaatcaaacgagcatttccttacgcatatgcagcagacttagacacaatggctggtgatcaaagtaaactaggttgttttagtttaggggtgccagacgaacaatcagaccctttggtatataatctatacgggcaataccgttacggatacaatgctaaaggtcgtcagttggactatgatgcactttacgatgcactaatagctatggctaacgatttgcttctccgtgaccaaaaatgtaagatcggcttacctatgattggcgctggacatgctggaggtaattgggctatcatccaagtgatgattgaagaaactttagtaaaaacaggtcacgatgtgactatttataaactttaagaggagaaatattatgggcgttttagcaggtattcttgcagtgattattgtgtttgttattgcttttctgattagtatgattccagcatggcttctgctgctggcttataactacgttgttgaaatgatgggtcacacagatatcactgttcctgtcaccttctggactgtggtgtgtgtggcatttatcatggctgtactacgtgggatcttttcctcaagttcttcaaaataagtgttgacaaagttttagaatagagtaagatgtatcacatgaggcggggagtggttccccgcaaagagaaaggagaaaataatgcaacttggtttccccattattgatgtagttgttttggttggtattttagcattgtctattggtatcgatttctttggtcataaagagaataaagaaattggaattaaatctgctttgctttggtctgttttttggattgctttagctttaggatattacgcattcatttgggtagaacatggtaaagagtttgccgcgcttttcctaagtggttatgtgttggaaaagagtctttctgtagataacctgatggtgttcatggcaatttttgcaagctttggaattaagagcacacatctccaacataagattttactttgggggattgcaggagccattgtcttccgaggtatttttgttgcagcagggaccgctttgtttaaccttcattggtcggtccaagtgttgttcggtttgattgtagcttggtctgccgtggctatcatcaaaggtggtgacgaagaagaggatattgattactctaagcattgggctgttcgtttagtaggtaaagttctgcctgtgcacaatgcactggatggtgaaaagttcattaccattaagaatggtattaaatatgcaactcctgctcttctttgtgtgtttgttattgagcttacagatattgtcttctcttttgattcagttccggctgtgatcggtgtgacacaagaacctctgctagtttattctgcaatgctgatggctgttcttggtttacgtgcacttttctttgtgttaagtgtagctgtgaaatatctggttcacttagagaaagctgttgctgtggttctggtgtttgttggtgctaaacttatgtatcacccgttcggtgaaacacttcatgccaagtggtcattcctcccgcatgatatttcccctaacgtaagtctgatgattgttctaggcacattagctcttggtgttattgcaagctttatcttccctgaaaaagaagaagctgctgtttaatagatgagcctccttcgggaggctattttataggagagacttatgaataattttgatgaaattcaagaacttgtttgggatttatctggtgatgtggattacgttttagattatctttcaaaagtttaccctggggtaagcgttatgatcgtgaccaaagatccaattaatttgcctcctaacgtggtagcttgttgtgggagtggttcaggggaagtagagcctgtatctcgttgggaattaactaaagctgtttcaaaacgaaacggtgaagttttgaatattgatatcgaacaaatatggacttctccagcagccaaatggaatgatccgaatggttgggatttgttcttctgggataatgaaactgatgatgaggtatttttaaaatgaaatacttgttggttgtgtttgaaaatatttgttttgcaatagggttgtttgttgtaatttttgtattgacaagcgtaatcaacggcagtaaagtgagtttataccgagatggagagcaactttattgcttctctggagatttcaaacacagatgtattaatagtcaaaatgaggaaagcaaatatgaaaaaactagccctgattttaattggttgtatctttttaacagcttgcgaggatgaaacaaatcctgcacaataccaacaacctcctcaacaatacgctcaacaaggatctgacggagatacaactattatcaacaatggtggcggttctagtagtagcggtgttggtgagtttgctacaggtatgttgttaggtcatatgatgagtggcggtagtaatcgtagcagtggggattataacgataacagaacgattattaataagcattataatacacctaaaccgaaaactaatttttatggtaagcctaaacaagagataaaaacttatgaaaagccgagacaaacttttaaaacaaatagtagctatacaactcgtaaatcttacaatagccctttaagtaaaaagagttcatctcgtagcaagcgacgttaataaggagaaaaacaatgacaatttctattcgtacttccatcgtaggtgttaaaaggttgcctaatgctttgcaaatcacacgcgctttgagggatacattctctgctgtggacgagcataagatgcttgataatctttcaaattggccctatttccattattttaaaaatgaagatgacgagtttatagacgctgtttcttatggttatgatttcggtaaggaggtttctgctgaagaatttgtagctatggtgttcccaaacggtttgcctactcgcagtaaacgtaaaaaaggaccaaaaaacaagaaacgtgctactaataaagctaccattttctttactgatggcacacaatatacgattaaagattttgaagaggtatcttatacagcaggtgggaatctttctttctacaccatcaaaaaacaaaactctacgttcagccatgtttctaacatgattgaaatgttagtagaagatattgaaagcgtagctgtgttaggtatgcataaaagctttttaattacaaatatttcagatttcagtgcaagtttatctggccctgattttgaagccacgaccacgcatttcgcttttaaggtgtaagttatgcataccgagagagacttgcaactcttgtggtgggaagtgatgcaggaaataaaagaagattttcctgtttactacaacatgttacgtgaagcaggatataaagcttgtgttagaaacaaagtgaataactctttcggttggtgcaactaccgagataaaattgttgttgtaaattggcacttgcataagaactccacaagagatatgatcgtggatacaatgaagcacgaaattgctcatgctattgatggttgtatgggtaagtttagtaggcacggtgctcattggcaaaaactggcagcagaattaggggctaatcctaaagccacttccaaagagccgttgaaccacgaatataaatatgttcttgttctggtagataggtttgcgacaagatgtgttagaggttataatcgtaaacctaatcgcatgtacccaggtaagcaacctaatatgtggttgaaatcagataaagaagggaccgaaggaaaattatggttgtataaatgggacgattgggttaaagtagctaaagagtacggtgtttcaccatatataagagatgcagaagaggagaatgaagaatgacaagtccatttttaaaagcagctactggtggtcagaagaagcctgaaatgagcgtatttgctgcaacaacagatgcaagtaaatctgtattcgctccaagcgtgttagatgccagtcctgtttatgcaccagcggcaccgcaagaagttccagttttgccttctatgacccgacaagagatcgaagagattggggcagaggcaggaacaaccttgcgttccttgaatgccgaggtattaaaacatcaacgtagtaatcaaggcgatcagatgagtgaacgactcacagctttgattaaacaggcaaaacaacttgatccaaactctattaaacaaagcaagggtattactaaactcattaaaaaagttatgggtatgaaagaagacctgtttggggagtttgataatgttaataaccgtattgaagagttggtaaagaaccttcaaatggatattcgcaaagaagaagattccttggtatatcttgaaaagctacatcaagctattggacaatatgctgcaagtttgaaccgagatatgcagcttctaacagcaacctatgaaaaagaagaacaagctttggctgctcttccagaagatgcagtagaacagcgacaagtgatccgagatactttggatcttctggagattcgtattgcagatttaaaagcattgcgcctactgtgtgtccaaatgggaccgcgtgtacaaggtatgcaaaaagtttctgcaagcttaatgcgatctgcgcgtaacatagtaaataatgttattcctgcttactctgctaacttctcaatgtatatagagagcttacgtcaaatgaaagcagcagagacgcagaataacgttcttgatgaatttaacctggcaattaaactgggtagcgatcttgccacacagaatcagatcgaagcagcagcactggctaaccgtcaagtgttggatattgaaactctgcgtcacgatcaggaaaacttggttaagatgttggaagaaactaaccgcatcaatacggaagcccgtgctgctcgtgttgattatattaatgaagttcaatctctggaagacgggatggtagctgctgtgaaacgagggcttggtaaatgagaatccgtatctatataattgatgaagacattggtgtccgttatgatgacgaaaaacgattcgcaccgatgggaggcatatatcccccaagtgttctagcagtttgtcgtgattgttatatttatgaaggttgtcgtaacttccgtaaattcgaactccttgcagatattgacttcaatgaggaggaggagtaatgaaaatattacgagtgaatattgaaactgcttggtgtggatgcaatgaggttgtggatatcgaaattgaagatgatatgtctcaagaggatatagaagatcttgcacacgaaacctttcaggattattgtaattacgggttccatattttagaggaggaagaagatgtataatccaacaggtattcttttaaaacttacagtaacagaccctactcttttcatcaagttaacaagtggtgaagtttatgaagtaatgaaagatgatgacggtgggtgttatctttgtggctacttcctctgtgatgcagaagagttagaatccattgtaactaaaggtttttatgaagaaccagggttccgtgtggaaactacgcagaaacctgataccattacgttgactcgacaggaactgatcacaatcattgaagatgcttttattgatggctggaatacacaagataatgactggggtggacccgaagcttttctggaacatcataaaacttatggagactcttgtgaaaccactgtagctcttgataatctgaaggagggtttgtaatgggttgttttagctttttgtgcaaagaatcgggcaaagctgccctttctacttctttcgacggatcaccttgctacctctttcttttgaaggacggtgttgttattgaagagatgtatggcaactatgatagttatggttgtgttttctctggaaaggaggatgaaaacggcgttcgtgaacggttcgaatgggctatggattggggtaaggtttgtgatttgatgcattcctcaaatccaagggacggtatcgctatggtgctggaagagtactttacaggtgaaatccctacagaacaatctgacggagatcctaaccaaggttggggtgatggtgaagaagatccagattgttttttcagcacagaaaagaataagtttaaacgtgttgacaacccttatcacaaagtgcataatactctcattgaaacagaagatgacaagatggaaattgaagaagatgatcctcttgtaacacttcctataagtgagttacaaaagatcactaaggaagcttacgatttgggcttcaaaattggtagaggaggtatgcgtgtttagattcaatattcaagaagaaaaaagtcaggatttacctattgctttcgaagatcttgacgtaggggccacattcatgtattatgatgcctatgaagacgcagggttggatacctaccgtaacttggtgttcattaaaaattccacaaacagtgcgatctgtattaatggatcactagcacaacgaggctatactcattgcatcccaccagatacaaatatggtggaagtagatttagaagttaccaatattacacttaagaaggagaaataatatgtccgagaaaatcagcctgaaaaaacgtcaagaaaaccttgctttctctctgaaaaagaataagaacctgaacgaaggtaagattgttcttcgtgtagcatgtgctctagatatttctggtagtatgcatggtctatattctagtggggtagtgagtgattttgtaggtaaactgcttccttttggtatgctgttcgacgataatcaacaaattgatatgtgggccttcaatacgagttcccaagagcttactccagcaacagcagatgtttacgatgattatgttggtaaatgtatgcgtaatgttcgtatcggaggtggaacaaattacgcccctgtaatggcggatatctacgatgagtattttggcactgttactaaatatattaccactaaacaagtggtacaagaggaggctccaaagaaaggcttctttaatcgtctgcttggtaaaactgaaacagtgtctgtagagcgtgaaaaagttgtagctgtatctgaaccaggtgctgacgttacccctggtgttgtattcttccagacggatggtgagaatggtgatcctagtgctgttcgtcgtcttatcgaagagaataagaataaacctatttattggttcatggttggcgttggtaatgagaagtttggcttcctgaaagaactggcaaatgcttatgataatgttggttttattggcattaaagatttagacttgacagattcagagctttatgacaaactcttatctgaagagttcggagagtgggttgttaaagtaggtgctaaaaaatgatcgacgagcttgcctctcaagtcatagaaaattattggcgcgagagagaagctttcattatgcgcagtctcaaagagtatttcattgggactgtgcgaatggaagatattgtaagtagagtagagattcatgtatggcctgattatgaagaatgggttgacaaagaaaatgaaaatgctataattcttcgtatatacaaaccggagataacattaggaggaagaagtgttaaacttcgaaattgatgagcaaaaagtaaataaacgatgccttaccattaaagatttggatgtaggagatactttcgtctatgaaaatgcatttgacgaaaatggtttagaccttagtgaaatcattgtttatttaaaagtagatgatggggaaaatgattattctgctgtagaactaaaccatcatgcacaattagtatcaatccaaaacacatctaagcttttggaagtagaattgactgtaactggtattacacttaaataatagaggagacaaaagatgactgataaaatttccctgaaaaaaggctctagcgaaaaaatttccctgaagaaagttgctccggctctgaaagaactgcgcctggaattctcttggaaatctgattctaaactagatctggatgtaagtgcactggtttgtcgtcacaatgccgctggcaatcctgaaatgctgtctgcacgtcatctggtttgctatgctaacaagtttgatcctgaacgttctaccttcgctggtgacgatgttcgtgatggaactggcagcatggagcagattgatattactctggctaaaatctctcctgatgcacaagagattgcctttgttctgaccattgatgatgatacaggtctgaaacgttttggtcatgcttctgaaggtatgcttaaaatcttcgactccgagcgcaatgtggaaattctggattttgacttcctggctcctgaagtagctggcaacacaatctgccacgtggcttccttggttcgtgaagatggggaatggaagctgaaagcatatggtgtaggtaaagctggtctggatatttgtgatgtggtgatggccttcggtggtaaaaaagaatggtatgaatgtccgtaataattaacaaatgattttgagggggagaaatccccctcttgcaataaagcctctatcgtgttatactagaaattcgttaacttaaaaaggaggcaaaatttgctgataaaaaactttgtaacagttgcgttactatccttaactgcttttacatttacacctgttgtggcgaaagccgaagctgcaaacgtaaaaaccgctaaagtagttcacaaatgtacaaagcaatataatgataaaattctattagcttgtgcaatttacgcagaaggtcgcggtctgggtaaaaaaggaatgaatgccgttgggaatgtggtaatcaacagaagaaatgatacctacttccctaacacagtcagaggagtcttgttccagaaagggcaattctcatatgcgaaaacttttaacgtaacagagaaagataagtggcaggaagcaaaagcaatagcggagcgtttactttatttagagaaacattttcctgaattgcggtctgttttagatgttactaaaggtgctctatacttcaagaaacggaatataagaacacactgggaacgtgatatgcgtcttgtttatcgagataaagtgcatggtttctatctaccaaaggggagttaaaatgcaagaagataatactgttgttgtagcaagttacctggttgtcggtcttactgaagaggatgtatactacacccgtaagcatttagaagcatacgctactttgtttatgggagatgaaaaggatgtagataaatatagcattgacatgatcaacgacagtatttatccttgtatggttcttaaagtagatgctgtgttgtgggagcgttttgcaaggactcatgatgctgtagaatgctccgcagtaatgtctcaaaacaacattttaggggagattcactgatgggagaagtcattgaagtcaattttcgcactaaacagaaagtgcaaaaatatgtcgttattaaaaacatttgcgttgtctgcttcaatacagtaatatacgactcacgacgcggagaaaacgaaaatgaaccttacattgaaatttctaagaacaaaggtcaatgtatttgtaagaactgtgcagtagctattaaggaggttgtaaatgaaaacgcttgggaccaatgaaactggcgagatctctcttcaagaagtgacaaacgacaaactgattaaactacgggacgtagcccgtgaccatctccaagaatcttttaaagaatatatggaagcacgagatcgtgtgatccaaatccaacaagaaatgattaaacgtgggattgtagctgacagttaatataacggaggggagaatgatttattatattactccctatctgacaggaaatatcggcaaggctatcaatgaagccattgccgttcttccagatgatagttgggtgtgtttaagagataccgatacaatgttcctcctacctgatcaaccatctatgcttgaagagcttgttatgtcaaacccgccttttgatgtaataggtgcaacttgtaacaggttaggcagtggttatcaacttttctctaaccaaattaacgatgatccagatattctaaaccatatagaatatgctaagattgcaagagaatatcataaagtaaatgttgaagaagttcctttcggaacacctctggcaggtttctttttgctttttagaaaatcattgtggaatgaaattcctttcgaagaaaaatctatccaattcgacttaattttctctcgcactttacatgaaaaaggaaagaggttaggattgatgcggggaatgtatctgtttcacatttacagattaggtcaaacagatcctcaacggtctatacatcatttattacactgtcatgatcacagtaaaactatttaggagagaaaatggaaaatattaaaatcggagtgggtgttatcactgtaggtgtccgtccacttaaagactaccttgtaacagatccacgtgcagattttcgagtattcaacgatccagatcgtaaaggtgtagcttatgccaggaatgaacttatgcaccagttttatgaagaaggttatgattattggtttatttttgacgatgattgctacccagtcatggagggatgggaaacatactttgtagatcaagctgtctctggaggatgggatttctttggtatgccggagtatttcaaagataaagttgttggaagcaaaggagaagttattctctgggaacttgcaatgtgtcaatttgcaatgtattctcgtgccgttgttgagaagatgggttatttccgtccttacactcacggttatggttttgaagatgctgaatggggacaccgtttgcaagctgctggtttgaatcatggtattcaggcaacaccttgtccacttcgtatcatggcttatatccacccagacgatgtttttggaaacaatcctatgccttttgctaacctgacaaaagaagagaaagaggctgctattgggattaactgggaagaatataaagcatctttagaggatattaaaaatgggaaaatccgaatttcctatgaggaggcactagcacatgtcaaataaaaaatggtctaaactaaaaattgatgcaaaacctcacagcggttgtgactatcatcgtattgttataccatttaacaacaataatattcaacctaagaaagatgttgttgtgtttaaccgtatccactctaaaggtgcagaatacgttttatctttgaaacgtcaaggtgttaaggttgttgtagacttagatgatttttaccaacttaatccagaacattatttagcaagtctatacgagtcaaacactcatgctattgtagctatggttaagcttgctgatgtggtaattgtgactacggaatatttggcatataagatccgtcacttaaataggaatgttgttgtgatccgaaatgcacttccttttgatagcggacaattcactttgtcaaatgatcgttcatccggtacccctgttgtatgggctggcggtgctagtcacgaaccagacttacgactagtctctaatactttcgatgatgaacttcttactattgcgggatatgaagcatcacaaagcatcacgggacaagagtggattaagattcgcagaaatttaccaaatgccaaatataaagccgctgtaaaagatttagataattacatgagtgtttacgaaagtcatgctattgctatagctcctttggtagataatgattttaataattgtaaaagtaatctcaaaatcctggaagctggagcaaaaggtattccaattgtttgttctaaggtattaccttattttaaccctgtagatgctcgcgctgttgcttacgcagagaataaagcagaatggcattacgaaattgttaagcttcttcgcaatccaaactatcgggaagatcgtggcttgttcttggctgaacatgttcgaacacattataatctggcagatgcaaacgagttgcgacgccaggttattgaaagtttgtaggtgaagtatgggacaaataattcttgacaatcgtaacggaatcaattatagtgtctgcgatttccatagggtagtgctgccttactcaaggcagttctcttcacaacctaaacacaacgttttcgtttttaacggagtgccaacaaaaggtaaagagggctttttagcccttaaaaacctaggctttaaaatggttatggatttggatgactctttagttattccaaaaggacacatgttagaaggtatttttgaatcaggtattcgtgaacatttaaaatggtttttagagcacagcgatcttgttacaactacgacacctgctttgcaagttgaactttcacaatacaatgataatgtgcacataatccctaatggtcttccttacgatgaaggtcaatttgtgttgacccgtgatcgctactccaaaactccattagtctgggctggaagtgaaacacataaacaagatttggctatcttgcccgattttggtaaagatcttacaatatgtggttttcgtagggattctgaagttttaagtagtcgtgaatggtctgtgatcagagaaaatgtagcaccaacttgtgtttatgaaggtgtaagaccatatgccaactacatggaatcttacgatggtcatcaaatcagtcttgcgccattgcttgataacgtgtttaataatgcaaaaagcaatcttaaaattcttgaggcaggtgctaagggtttacctttgatctgttctcctcgtgagaattatcatacacaagagtttaaagatttgttattctttgctgaaacaccttccgagtggaaagatattacggaatacttattaagtgctcccgatgtttgcatggagaaaggattagcattagctaagtatgtcaggactcattatcacattgatgtgttgaatgagcgtcgtagacttttatttgaggagctataatgattaacacttttgattttgaatacaccactcgtaacgggaaagccttagtcgtggaggtggattactattatgaagagcctaacccgctttctcgtgaaagtgattgggattaccacggagggcttttaattgacggtattcgtttttatgaaggctctacagaagtttttgaaaaagatttaaatgtaactgtagcagaaattgctttccaatttagaaagcacatggaagaactagaagcagaatatatgatggagactaacgaaccattttaacaggagaacaaagtgtcgcacattataactaaatatggatttgatgttgatcttagcaaggaagggcatacaggttgtccgcgctgcatgaaaaatggcaaggatcgctctcgtaataacctgatggtttatggattagattctaatggagagcataagggtgctaagtgctttgcgtgtgaatacactattcctagcgtagaatggttacgagaaaatggtgaagttttagaagaagaggaggagattgtgggttcggaatttaataaacagattcatcgtaaaattgttcaagaaacatcaaccaacccacgtggttatcgaggcattaaagaaaaagtttcatctttctatggtgttcgttatgaataccgtcaggaaggggatcagatggtagtagaaagttgtttatatccgactaccatcggtaaagaattggttggttttaagcgtcgtattcatcctaaagatttcagtaaacctattggtcaagtaggtaaagatgtagatctttttggacaaattcgttttgcagaccgttctcacactgtggtaattacaggtggtgaacacgatgctctggcagcttatcaaatgctgtctgaaaacttggacaacaaatatgagcttccagcagtagttagtggcacaactggtgaaaccagtatgtataaacaagttcaggcacaatatcctttctttgaaaaatttaagaagattattgtttgcatggataatgacaaagcaggtaatgaggcatttgaaaatattgttaaggtgctgcctcgtaacaaagtctaccgtatgcagatggatcttaaggatgccaatcagtatctggaagaaggtcgtgcaaaagatttcgtaaaagctttttggagtgctcgtctatacaccccagcaggtgtttatgcttctaatgtcttgtatgaagctgcattagaatgcttggaatccaagatgattaccttgcctccttttatgcgtgtagctgcaaacatgctgggtggtggtttagtagaggaagagattacagttatcctggcaaagaccagtatcggtaaaactttgatggtgaacgagatcaccaaacatattattgttgcgcacccagaacataccctaggtattctgtccttggaagcaacttataagaaatattctcgtaacctgttgtcctcgttcctgcatattcctctacacaggaaaacaccggaagagaaatttcaaatcttggaagagaataaagaaaaaatcctagatttttacgaaagagaagatggatcaccacgtttttatgtgtgtgatgatcgtggagcagatgtggagtctatcaaagaaaaagttttggagatgatcatctattttggagtaacaattcttgttatcgacccttattctgatttgctttctggtatggatgttcaagcccaggaagagttagcaacatggttgaagcgcatcatgaaagagtatgctataactattattgtcatctcacacgttaagaaatcggctaatggtggcaatgaacacttaacagaggacgatgcaatgggtagtagtttcttggctaaaggtgctggtattaccttggctttggaacgagataaagccgctgaagaccctgtagaacgcaatacgactcattgctttatcctaaaaaaccgtgagttctcagaaactggtccggcagggcagttttactatgacattagaactgcaactctgcatgacaaagagcagtatttcaacaatcctgctaatttgcctacggatagcaactattaagggcttcggcccttttattaaaaatacctgttgacaactttacggaagagacttatacttgtctcatcaaccaaacaagaggagaagtaaaaatgcgtatgacaatcgatctgaatgcaaaagttaaagctgctggtgctctgggtgctggtgttattaaacacaaacaagcgttggcatttgccatcccacatatgattgttgaggatcatcctgaagagtttgctgtaaaagttgttgagaataatgtctctgttattgaacatcttccaactggacgttcttaccgtgttgtttcttctaaaggtttccgtttcgatgtaagcccttctttcacaaaaggtgctggtcgtagtaaagtgggtattctttcttacaaatattatatcgataaagttgcagatggtgttgcattctgtcgtgtaagtgaaggtaaagtagatatcatctttaaaaataccaaaaacatggttatttctgacgatggtaaagttgacctcactattgagttcgctaacaactaaggaggactattgagcaagttgaacacacttgatcaatattacactaaccccgcttatgcggggttcttatgtgataagatcaaagaaatttttggagaggaagctaactacgtagaaccatctgcgggagaaggggctttctcttcttgttttaaaaacataacttcttttgatattgaccctaagtttgaagattgtattgaggcagattttttagctcaatctgttgagtctgttggtagtgtaactgcttttgtcggtaatcctccgtttggaaaaaatgctagtcttgccattaagttctttaaccattgtgctgaaatgttggataacgaaggtggtatttgttttatcttgcctcgcacatttcaaaaaatcttttttgcacacaaactaaatccccacatgcatcttctttacgaagaagaatgtccaaaaggatcttttattcttcatggagaaccttgggatgttccttgtgtattccagatatggattaaacgcaacgagaagcgttctcctgttgttttaggagaaaatcgactattcgtattaggcaacagtaaagactacgacatagccgttaggagagcgggagcaagtgcaggtaaagtgttatcagaagaggattctttatctgaagcatctacctattttttaaaagccttgacttctgatcttaaaacgcgtatcattgaggcttacccgttacttaaagctgaagctagtaaaactgcgggagtaagaagtttaagtttaaaagaacttcagtgggttctggagaacattagaaaggaggaatagtgagtaagtttacatgggatattgaagcaaacggattgttgaataatgacactattgattatttagctagtccttatcgtctcaaagacacatttcaaatgcactgtattgtcttagagaaccatgaaacaggagaaatagtagctttccacgatggccctaaatacatctttgatgggcgtccttatgaagagtcagatggtaaatacacatatgttcttaaagagtatgtggcgcaagattacacacattttcaactaaaagatttcccagattttattaatcatgaaggttattttaaagacaagcctgaatatctggttgaaatggctgtggctcacaacggtatcaactacgactggttggctacaaaactgttttatggattagattacagtgttaaatataatacgtggggcaaaaataaggtaaaacttattgactccttgattttgagtaaagtgttaaacccagatcgaaagattgagggcaaatcaggacatagcttggcagcactttctcgaaaagcaggtggtgatgtgaaaatagatttccgtaaagagattccagaagaagatcgatttaagacttttgctgcggatatgctttattactgtatctatgacaacaaagccaatacgagcgtatataattaccttatggaagaatggggtgattatgataaatgggaaggtccatttgaacttgaacaacgtgtagcagacattattacacgccaggaacaccgtgggtttgcttttaacatgaaacaagcagaagagaaccttgcttttttggatgcagaaatggagcgtcttcgtttactcgttgagcctattttgccacctaaaccagcaacaaaaggttatcttaaagcttttattccacctaagttgcaaattaaaaagaacttagagccaactaaccatatgatcaattttgctaagaaacatgatggtcatttggaagagagggatggcgattggtggttagttatgttggggaaagagtggaaactgccagttgcagcagatgagcctctgtgtgatcctaacatccctgcaacattgaacgatacaacccatattaaaaactggcttgtaggattaggttgggtaccgtctgaatataaagataaagacttgactcttaaatctggcaccaagattaaaaagagcgaggaagaacttcagaaaggtattcgagcttacgttgagcaaactctatcttctaatttttgtcaggatcgttgtgatcatctggagtgcacaccggaaaccctggaaaggaagattcaagagcgtatagcaaaaggtaaaggtcgcggtcttaaagtattgactaacccatgttttaccaaaggtcaggaaaaagaaatttgtcctaacctgcaagctttgggtgagaagtttccttatgctacacagattgtagaatacttaacgtataagcatcgtaggaactctattttaggtggcggtgctgattgggaagaagatgaagaagatcaagaatacgataaaggtttcttagccgcagtaagggcggatgggcgtattcctaccccagccgctacatgtgatgctgcaacctctcgtatgaagcatagatctgtagctaatattcctcgtgtcacatctttgtttggatgggagatgcgtaacttgtttggtgtagaggttcctaaatacttccagatcggctacgacttcgactctttggaagcaaaaattgaatcacactactgttggcggtacgagccggagccacacgaatattgtaacgctctgttattagagaaacctaatgatgttcacacaatgatggcaaaacgtatcactgaaactatcggacgtaaatttgaacgtagtcctgctaaatctgttaagtatggtattacctatggtgcacaagccgacaaggttgctaaaacaattggtagcgataagtttacaggtcaattggtgtttgatgctttctgggaagctgcattcccattggcgcgtcttaaagaacgtttaaacgatttttgggttaagactggtaaacaatatgttttgggtattgacggtcgtaaggttcctactcgttctgctcacgcgattcttaactctctgttccaaagtggtggggttatctgtgctaaacgtgcaatggttatttatgacgacatgatcgaagaagaaggactaagtgttgacttctttaaagatgactggaagaataaagatttcgtgcaacaaatgattgcatatcacgatgaagcccagcttgaagtttctcgtaaccttgtagaatttaaatggttttcacctgaatcattaggttggaagaaagtagacgatcctgaagaacaaaagaaaattgataaagaatgccttgctaaagtcgaagaatggaagcataataaggaaaaagagacagggcaaatttgggctaacattcatgaatctcctaaaggtggatggtttacagcctatagtcgggcaggtgaacttgctgccttagctgttactaaggctggagaattttacaatcttaatgttcagcttactgccggatatgatgtgggtaattcttgggcgtcctgtcactgatctaacatattgttttataaggggcttctgcccctttataggaggagatatgcattacgatttagattgtcttggtatgccttacctaccagatgaattcgaaggtgaagaagaacgtgaatgggacgatggggatatccgttgttggtcttgtggtaaagatatgtcttatgaacaacatatgcacaatgatggtttctgcatacattgttataatgaaattgatctggaggaagattaatgggaaatggtatcatgatttgctataaaattgtgcgtattgtcggagacgaagttaccactcggtattttatgatagagtctcaagccgatgaggtttacaatgactggcttgaaaaaggtggatacgattttgtatctaaagaaactttccgtttagaagtataaggagaaaacaatgcgtaaattagtgagctttaatgtttcaattcttgttgatgaacaaaatgaagatgttattaaaggtcttgcaaataagattgcaggtggaatgacggaagctgaagtgtgggaagaatgtgctcgactttatgaggaaggtgttattgaggtcttaggggatgagattaatatggacgatgttgtaaaagtaactgtggagcgtatcaatgactaaatcaaaacaaatcaatattgccaaagaaattcttaccatattaaagatcgctgatcctaattctgtcttggctggcggtgctgttcgtgattggcgtattcttgatattgctggacgtgatctggatttctatacatcccttcctgcacatcggactgaagaacaaatacaaggattaattgcatctgctttaatgggaaccaatattcgtaaactagggaacaaagaggcttctgacaattatgaaggtgttactggtcttaaacacgtttttgagtttgaacgtgatggtcagctttgcaaccttatgattatggaaccaagcgtaatgacaacctttctaaatgagttcgatctgtccatctgccaggctgcatttgatgggaaggactgttattattctaaagattttgaaacaaccttggaaacagggatctgcttcttgcacaaagatgttagagaagatgccaaacatgttattaaaattgctacacgtttcccatatcttcgttttgtgaaagaggttaaggttccacgtccacctcaagtagataccagattggaagaagatgatattccttggtaattaaagcttgacaatattaaacattttatgataggatagcattcaatgattgaacgttctaagatagctgttgaaactgtttatggaaactataccagtttgacactagttgagaaaaatgttgtaggtcttgacaactctttttattttcaaattttggttattcaaggtggaaacaagaagctaactgctcgtgtcgaaacatctgaaataattgaaaccactgttacaaacaaagaattcatttcactgcaagatacaatccgaaacgttatagacgaagaaggcaaagctgccacaattaaactcggaggtttcccagtgcaaatagttcattttcctaatggtgaaattggagttggaataggtaaaggtaaatacctgttagagcaagatgatatggaaagactccgctctttctgtgcaaaattaatgtaatgtagaggagataataatgtccgtaattaatactgaaactgatcgtaaaggcaaaaccgtaacttatgcaactggtatcgtagactacgttaaactggataaattcgctgaacctaaaaccttccattgggaaggaaaaacgattgtttcaactcaccgagcttctcttgttctgaaagagaaaggtgctaaaccagatgagaaaggtgtttggatttctcttggtgacattgaactgaaagaaggttatgaaaacctgcaagtcaaagaaggcgataattggatcactattgaaaaaggtgtagaagtaagtattgatattgatcgcgttgataataaaggcggtaaaacttactacaatactaaaaaatctcgtattacagtattgtctaccgatggtgttcagaagtctaacaaatcttctggcaataaaggttccaacgatgctgctcctaagcaatcctataaaaaacgtgatactgtaggtctggaaactggtcacgcagtaaatggtgcactggaactgattcgtaatggtgttaaaggcgatgctttcgatatcgctggggttgttcaatctgcaactgtcacgctgaaatcggaaattgctgaatctcgtggcgttgatattaatgattatgatcttggggcatctgttggtcacgctatcctcaatgcttgtaaagaccatactattaagactgtatctgtagaagatctgattgattctgctcgtaaagttctggctctgtctgataaagttgctgctgccattcgtggttccgcagaacagcctaagaaagaagagaagaaagaagttaagaaacctgaaccacaaccagaaccaactccaagtaatcaagaagaacctccaatggacttcgatgatgatattccgttctgatcttattgatttataaagagtttttataaaagaaaggagccaaatggctccttttcttatttataattggaaggaatgttgttataatcagaaagttttgtgcaatacctgaataggtaactattagctactccgcctaatgtatttgttaatgttgttcctgttcctgtaagatttgtgcatccactaaagaaagaagataatgctccacgtttaccacaaacagttaaccctgggttgtcattaaagattttattaaaatccgtaagtgctaaacaaccttggaatgtagctgttaaagaggtcactttcgttggaatattatcaaacattcctgctggaacttctgcaagagaggtgcaatctttaaaggtgtaagaatatcttgtaacttttgatagattcttaaataaatctgaaggaatagatgttaaagatgtacaaccttcaaacgtgcttgacattgttgtcagtgaaggaaataaagcatcactgaataatccttcagggatagctgtcaaacttgtacagccagagaagataccacttatagatgttccggtgtaagatgtgttactaaaacaatcagaagttattcgtcttaagttagcacaaccttttaagaattgtgaggctgggcttgtagaataaccaattctgttgccgccccataattctaaaagatttggatttgcttccatagacacatcaacactattcactactgaagcacttgcaaattcaatagccagtctatattctcctgctggataactatccactgtagagaaagctttatttctaaatattactttatttgaaggtattacaacgtcatccaccttaactacaaagttagctggtgtgacagtacccgataaaattgggaacgtaattgccccatctgccgagtggttgaaatatccgtttagaaccgttaagctactactcggagatggagctaactcctttcccgcaacagcattcaccgttaaataagaactatcagaagcggtaacaacaccttgatatgtagctgtgcaaccaattctacaaccaccatcaccaacaccagttattacaccagttgtaggatctactgtagcaacattaggatctgtagttgtatactcaacaaccataccaggtaagcttgctgcccctgttggcgttatttctgctaccattgtttgtgttgttcctacatccatcgcagataagctaggagtgtaaacgctcaatgttgaagcatatcctccagaggtatctgataaggttgttccttcagcgtttaaggctgtcatacctgcatcaaaaccaccttctgcatggaaagtcattaaaccagtagatgatattgtcatgtttgcaggggcagaagaagtgtatttaatattgaaaagtttacctccttctgggctaacagaatgtgtcaactgtttagttccacctacaatacaaattcctaaagaatctgtagttaagccagttgccagaatatcacccgtaatcacggagcttgctattggtgtgaatttattatagtttagaccactattgaacagatacgaggctttcccatctttccaaataccggaataatacatatccccattataaacgaaaaaagttccttgataacctaattggttagtaacatccatattcagcgtatgaacgcttgtgagcttgtttatatcccaagaacccactccttcaggtttaactggttttcccacttggagtggatctccgctcgcagaatcataccccaaacctaagtttgcagtgtagttcttaccaatagcccacaaggaattattgatcccaacgaaaaaagagtggcggcttggtggaaacttccatgttgagaaacctgctgcaaaggtatccagtttagtgtaggttgtttctgtattagatctaacatataagtagtttactgtacccgtgccaccggaggtatctcctacaactattgtctgggaaactgtattgatgtaatctttgacatacactgtttcagtagcggatatcttagcattgacaaaagctgttgttgtgttattattacctgctaaacctttactagctccacaaacacgcaaggttcccacattgttgacataagagcattggttgtaacctgcatcaaaagcaactgctgcacttgttatctgacgaggagtagaaacttcgttagtagtacttgcacctaaactgcctactgtgttttgacctgatccataaagaacaccactattcattaaccagagagtgttgttatatcctcccttaacctctttaagattggctaaagagatagtccctgttattacagatggaaagctggtccaagtagttacacatgcgtccgtaccacctgctgctatagatcctaccaatcctgctgtcaatcctgaatattgccatcccccactgttagttttaataacaaaacaacgatctgcggtaaacacatccgctactacagtagaagctaagtggaaattattgaatcgtgcactacgagttccatctgcaatctcaccaaaaaggtttgcgccttgagtaaacaatctaccatcattaagcaaaattgctaaactgctgttagaattaccagcattatctgggtaatctaaagtgattacttttttaacaatacctgctggcagaggaacgacaccagtgctggtcataatagtaaatattggaattggaaacatttaaaccctccataaaaagaaagcccctttcggggcttctgttatcgtgttacaacaaccatgtccacgatatcacccacaccaggatacgttaattgcaaaattgttactgcgttagcagtagttgctatttctgtatcatttagtgctccataagacgcatctagtgtaaccgtgtgtcccgttgtatcttgaatcaaataaatcattgctgaaaacgctttaggttttgtagagccagtgacaccaggccatgctccaactgtcagagcgtcagttactgtaacaacatacatattggaagttccagaaggagtccaagatgctccagagttggcagtagttgtttcaagttccatgaaacctgctgtaactctagtcggtgttatagcttctgtaggatctgatcctgatatttcgatccagtttccatcagaacgaccatacgtcaaaccatcttgaggggcttctggtatactaggaagagttcctataagtgtccacgtagaagaatctgttttcttaaacactgctccagtgttagtattgaaggcgtaatcattgataacaccgtctcctgcgactggattagttgttgctacaaaccactgtgcgccgtccttaccgttggccccatcagtaccgttggttccgtttgttccaggatcacccttatctcctttagcccctttttcacctttgtcacctttaatacttcccatgtcaacaaaagatgcggaattggtgtcttgatcgtaagtatagatgtaaagattactagtgtcttcagttaagtaagcatcaccatagttattgtaaacagggagagcttcactgttcggaacatgacctttagctgtaattgatattcctccaagactgccaacattcatccattccgtagcatcttgaacccaaacatacaaatcattattaattttatatgcatcgcctacttctccagtaggaggaagttcagcttcactgttcagaaccccgataatctctaagcctgatcctgttgcacccactggtccttgtggacctagttcaccttgtggacctcgctctccttgatcacccttgtctccttttggacctttaagtgaggcaataaaatctacttctgtcccttcattaccagcatccagccaactttgatatgcagatttcccttcatctccatctgttccatcaagaccgtctttacctgcctcaccttgtggaccttgaggaccaacaatattacccatgttgacccaagctgtcccgttagagacataaagattaccattgatagaataagcttggcttacgttttcagagttaggttgtggtaagttacttgtagaaggcaaatccccacgataatcaaaaccgttggcgttaacaccaggtgctccctgtggccctgtaggaccaacatcacctttatcaccttttatagaggtaagaaaatcataaacagatcctgtattaccatctgccaaccatacctcgtaagctgatttaccttctggcccaagagaagaaataagtttccaaacagaaccatcccaagcgtagattacatcacctacgatataaccatcccctgcaacgttacctgtggatggtagttcactagtgcttgctaacgatcctttaatattaagttgagcaccagtatcccctttcaaagaggcaagccattgtgcttctgttccaacaaaaccattatcaagtgcgatctgataagccgatttaccagcttcaggtattgtttccgacagatcatcaatttgttgctgcattgccgtaaacttggcattgataattgctatatcatcagcggtcccaacactttgccatgcaagggaagaaataccacctgtggtggctggcgtagagtttgctggaactactttaggaaaagttcctttccattggtaataattcccttctgctgctaacagcaaaacttcacgggaattacgtaatgtaaaacctgcctcaaagctgcgacgttttcgataagtagcttctaccagatcggctatcgcactattaggtcttgttaaattcatttatctctcctatcattagaccattctattggaagaagagggggattccccctctattccattaggttgttggcacatctactactggcgttgggcgaataaccgcagtattacctacagaaacattactggaatcattaggtatggttttaattctaactccagcacttgtaggcgttacaatgggatctgcaatgtctattttaactgggttagtccacgtatcattatcttgataaggtgcacgagtaataactgtgtccaaatctgctccacctacgccagcaccaggaatacggatacgtgtccctgctgacagagggtaatgatacaaccaagaagcatttgttgctgtcccgttaggagtcaagttctgtgtcaggaaggtatctcctgcttgtgtagctcgaataccgaaagctgggatatctggaatatatgccccacttgttgttacaaggaagaacgtattatccgccttgactatcatatcacctttgtcaaactctttccctgttgggaaatgcattccatcaatgtttcgactaggattaggctctaagtaataaactttgcccgttgtccctacaatagctccaataccacctgcgatattggtatacatattagcttcaacattacagttgacacgatgaatatccgagtaacggcaacggctccactgtggacgaccagtaaccatcgtcttgccatctgggtaacagtttacgttaaccatttcaagaacagatttttctctacggaagaatccatcataaaatgtccctggaccagattcaggttgaccaacacaatcgaagatattacaggtgatgttagacattgaaagccaagaagctataccgaacaactgcaatgcagcgcaacgaatattttcaaagctaactctgttttcataacctggcccagcggaaactcccgcaaaaacacgcccagttttggtgtgaatgttacgaacagaatagttaaagcatgggtttgaaggtctaccctgtgccggaactaccggattaccctcttcgtccagaactgcgttagttccccactccaaataaatcatacggatattacttgcaagtgtgctaccgtctgttttaggctctccataaacaccgtcaatcgtaaaatctttacttccatacattacaactgcgccagttgccaaacctgtcccaacagatacggtctggtctggatcgccatgaatgttttcgatagagatattacgaccaacttctacgtggacaatctgacggcaacgttttgcaaaaacgttacgaattgtaatattcttgcaatattgatcatcagggattccgtaaccatatggtcctttaccagctacaccgataccgataccccagttggacagaggaataggagcgttagtcccatcgatattagacaggtaaatattctcaatcacacaaccatcatcataatgtttattgatgacgttcagttctatcgcatcaccttgcatgtccatgaaggtaaggttacggaagacaccgcgtgttactggttcccctgtcccttgttgaaggatgccatataacccgtttttaaaagtacaattatcaatttcaaaagtacctgatattgggttgccttggaataccaagctaccaacttgacctgcgcgaggagcaccgtcccacaaacaatcgaacatgcgcacagcaccaccccaatacataacacgagagttacggaaagttgtgccttctactgtagcattctgcacacgatagtcgcgttgaccacggaacacaccaccgcgcaaagtgataccttgaatatcaaaagagcttgtcgtgaaagtcatatcgcaaccgtaagcatcgagaatcttacttttcgaagcttttgctgctaacagtgccgcctggaaagctgcttctggtgtggtataggtagttgtattgttagcaaattctgacaagtagacaacatcgaataaatcgttgtatgttccaccacgaggggttaacagtccttccgtattagttagaacccatgcgtttggacctaatccaccgcttgtctctaatgtgtcagaggccaaaatactttttggtaaacttccagcccaacgataacgaccagcaccacgtccaatcgtctgatttttagctgtcagagttacattattaccaaagcttgtaggcaaatggacaaactgtccacgttcaacagctagtgcagaaagatctacagaccctccagaataagtcattactccagaaacagacaaagatgttaaagttcctgtgatgttaggaggtaatgtgtaggaagactgtgctaccggatcatacagagctttaatccctgttaacgattgacctgctgttgcgtaagccacctcatcatctaacaaaccatatttacgagcaatagtttgcagagggactttatccatgctttctgttagttcttccgtttttgtttctaaactaacaagacggttctctagattaggatctgtatctgctgaagctacgattttaccaatataagcgaaaagcttgtttggcagtcctgttgcaggatcgtgtcccaaaggttcagataagatgatcttactatcttctatacggaaagcttcccccagaacctgaataacaccattgatcgtaacaatagcactatcaaagatgtaaggaggactgattactgtttcataaccagttgcaccagaatatacccaaggtgtgacttctgttgccaattgttctgtcttcaatccccacaatacccagttgttatctcctactggagtaccttgcatagggattgggtttaccaatgtagcattaggagcataataataaccgctcaataccccattctgaaaataacgtagttggttaaaaaccgtttcatttgtgccttgcgcccaatccaatgggcttttaaagaaaaagttatcaacaagggcttttctcagggttggtatcaaccctgattctgtctcaatactttctgtcgcacttccattaataacatcatgaagttgattggacgagaaaataaccagatctacgccctgttcaagagacgttgctggatacgttgggaatgttgccattaattaccccttattctgtgtacgtttaactatgtcatctagcatcatttggatagtcgcattcttgaggtccataatcttttcaattttttgctcaagagtgttattatttgccattaatgtagagttaatttgatttagcatctgggtaagttcggctttagttgcataagttgattcaacaaagaccaacttcttctctagctctcgacttttctcctccatttcatcaagatcagagccgaatttatccattttttgtttgaaaaagaaccagatgactccgattaaagggattattgcatcctttgcaagtccccatgcttcttgaaattccataatgatcccttattccgttggtaatgtaggccaatctatagtgtaaggatagtctttttgaagcggaacacggtttagtttaaccacataaacttgaagatttttaagataagctaattcatcttcagtagccatttctaaatctttagcatactgtaaaggtgttataagtgctaccgctgtttcttgtaaagagcgaatttttgcactagcttgtgtcatcaactcttcgcgggaatactctttatccttaagagtcaaagtttctaaatccagaaccttgtttctaatatcctcaaaagtgtctatattaaagtccacttgtgctatagaaagatcttcagggcaaagtaatgtaggatcagtgttccaaaacacaatacgattttgatcgtcaaaaacgactttcattttatcgttattgaattctgtgctacgtaaatcataccaatcgtaaccttgatcgcaacgaagaaaaagaatgttctctacttctaccaattcttttagttcgggatcttcgggaatatatcttttaaaatttgtaaatatcatttagcctccgatatcgtaccagtttccgttaacgttgcgctggaggcgtttgaaataaaccccaccgatgttatccgcagaactacgccctgtttcctggacaataataccagaaagaacataaccagaagaacagatataagtccaagaaacttcgttccctggtggataatacacgccttggctagtgtaacgaatatctcttacaaaggttccgttagcatagttgtagttgtttgtaatctgactttgatgattactttctacattgtttatacgggaagagaggttgctgttaacactattcaggttgttttgaacagtgttaatattcgtctgcaatgtgttctgaagagaagtcaaacgagaattcagataatctaagcgaactaaagcatttccgttagaagtatcttgtccactgtttgatcttggactgtctattagaacataacctgcatcatataaacgaacacctgctgatacactacctgtgctaggatttctacgataaatataaagctgacctccactttggtattcggcagaaatctcaccattagtttgtcctgcaaagtttttgaaaaacagtgtcgcaggatctgttgtggaagtaccgttaacataaacatcaccaccataaatagcaccacccgcatcacgttttaccaacacgtttggtgttactgttgaactagtagcatctgtaatgtttgccgacaagtgattatgactgttatcttgaacttggatagataaagtaacttctttattaccattccaagtagcctcaccagtagcatcacctgccattgtaatcttagttccaactaaaggcaagtaagtatcaggtaaagttggaagaaagtcatccaccaaatagcgaatccataaaccgatattattaagaatccagttcatttcttcgcaagtcatgatttgaccataatccataccaactgtgcggatggtttctttaggtctgattttgtttgtgtttaaagttcctgggagcgttacagtttgtgtagcccagtcaggataatatattggcttcgccatttaaactccttaatctatttgcttgagaagcgtacacatgcgtcctgtttcatccccagtagattccatgtacagggaacctaaaccaccaataccatgaggcggtttggtagcataataagaactaaaaccaaaagctgatcctgcttttgctccaagacggtagctactaataatagggaaaacacttagcaactgctctaaagcttgatcagattgaagacacaggttgtagaaaaacacatcaaaggttttattcaaacctacgtaagtattaagcgtttcacgctctgttcctgtgaaacgggcgaagatatcaattatgtcaccacgagtccccgatgaggctacgcggtaagccctgatttttaatattgttctatactcttcatcatcagagccattacgttcgatccccatttcttctcctaagatatctaggtatttgcctgtggctgtggagatcaagcggtgtttagcaagatagatcattgcctgatctatcttttgcttttcactggatacgatgtagaggagcttgacaatgttaggagaagatttaaaatcttcaatcagaaggtttttaacatcttcattgatactggcccaagttgttacatggtcaacactcactgccatttttacactccatactcgtaagaaatgttatcaaggtcaaaagaagggacaacattataagcaggaactatattattctctgtatatttactgtcatcctcagtattaagtttaacataaacacgaagagaagtcaagcgtccaaaagcaagtgctgcataaataacacctttgatttgatcgttcgttactgaagaacctaactcaaagtaagcttgcaggttttgtagagcttctacgatgttttgtttttctgttccacttaaagttgttccgttgacagtttgataaacaagttttacggaatacatagcagaagcaccaggagagaagcgaataatttcttgtcctccgtcttctgttgtaactgttatgttagttgttccgtgtgtcaaagtgttgattggtttggttttatagataacatttgcaatttcttctgcactgccaccataaactatagtctggaaagtcatagctggagcatgttcttgatcatgtaatgtagggttatcataaatcttcaccttagtaacaccatctgtaagtagcagagctttcacaatagctgctcgtgtccctgcgttagcttcatcaacgttgtcattgaaacgagagcggaattctgcgtctgtctcaacatcccgaccaggagaaaatgaatagaagttaccactgtcaagataacctgtaaaggtagaagatatccctgtaatagactccacaccaacaggattgtatcctttctctgctgcaactaccggaatctcactccacttggttccaaccggaacactagcataaaatttaataggagttaccagccccactggagtatcactggttaaagaagtaaaaccaattgataagttacctgaatcagaaaatacaagactacgatcagcttctgttaaattcgccttgataaaagtttcaagatctgttataaatgtagaagaaccaggtgcaagtgttttactgtttagtcctccatccaccgcactttgtgcataaaaagtaatatttggaacacctgtagcagttgcattagcacgagagatttgataagctgcaatcttagcattcaacatagttggtgtgctaacatagtagttcatatcactgtcagagttgaagtaaatatccgtgcctagctcataagtccaagctgctttgttattgctcttaacatatgcataacctgtacttgctgctgcccctgctcgatagaaaccgcgtttagaaaagatttcgtcaagataaataccttctgcaccatccagtgtttgagtattataaacaccttgcataccttcccacaatgtgtaatctgtgtatgcaattattctaaacagttgcccgatcacagtatcttccgaagtggctgcatctgcaccaaatgtgttttttactgctgtcatgtagttgtttaaaacatcgtcaaaagtcgggattacaaatcccgcatctgtcaaaccatattcgtccattagtcatcctctttagtagattgtgatctgccccaacgtaaatcaataagatttttcatgaggcgagaaggggttatcatgaaaacaaaagcccaacccatccattcaggaatttctttttctattcctaaaaagataaacatgcctgtcataacagacatgcctacagcgttccagactttcgtagagcttaccccgttgccggggtaagtgcagaacattccgcgaataaattttttaaaaccgtttttctctttagccatgagtacctctaagtgagatataaagattgaaataccacctaagctgtattatttactttccagggttgcaagtctttcttcaagagcttttaactttttacgttgataagccatctcaataaaaaacatttggtcagcgcggataccccatcgcgatcctgctggaattatttcgatagtttctacaacatcttctacgattgttactggattaccttcttcatcaattacaacattcccttcatcatcaaataaagggatttctttttgtcccgtaacaacactatcgtacatagcagggtattcatcataacacagaaaagcatacttacaatcagtgctattttcatccattaaaccataattaatcagaatatctcttacatcttgagctataacaccaaaatgaatacgcgcagaatctcctttctccgcgacagcactaagccattggtaagagattacatgaatatcttcccatgctttcagtaaaatatcttcaatctctgtaggtttagtttttaatgttccatccgaagtattaatggttcctgtagatgcgtacacttctgaccatctgttagacgcaccgcctaatgcaacttgtccatctaaatacggcatgaaattattttgggtatatgctcctcctggggcatataaaactgctctgtttggatacgtatggtttgaaccatatagttgtaatagcgatccatccgtaacgtaaggagtttctcctccacataggattagtcgctgttcagaaggattttttattgggacattttttgcaacaaccccataatcaccgtctaattttattctaacggcagattgttgcgtcccatcatgctcaaggccagtaactgttaaagagtatacttgtacatcattatcaaaagttacaggggctgagacatgccttaccccatctgtacccatagatacaggcaacgtacggtttggcgtagctccatatttgaaatcacgtgatacaaaatcatgttcggtaatctttagtctataactatataaatcggctggatgaccatcatgcttgtaccataattttttactattatctccaatagaccaaggagataagaaatcttcgcctccaaagatataatataaccaaccatctttaacacataccgaaccaacaccacatgcagaattaacaatatgcccttgatacatctgatcagtgatattaaaccattgaacatcggataaagatgtaggccatgaagatacattaatcttgcacataaacgttcgaggatacgtgcctttatatctattatctagttcttctccttcccattctccgaatgaacgttctgtaccaaagatataaaggtagttaccaacttttgcaaatggtaagtttgtatgatgaacgttgttagggaatcttaagtaattccagttttctccaaggtcggtactcattgctaatgtagaaccagcagcagaagttgaagtacccctagtagttaaatataaaattccatcataatatttaatgcagggttctgctgcattgtcagcatattcttgactaattgttctacgagtaaaagaacttgggtcagagtaagcattattgaaatacaaaatacctaatcttcttggggaaacatcaccattgtgataaccaacagcaaaagtgtaattatcatcatctataactgtaaaactatgggtttctgtaacacataaatctgcattagtagaataggcaacatctggtaattctgtaatttcccaagggctatcccagaaacgtgttccaaaatgccaagtagttcctgtattatctatattagatgtggcagcactcgctaatgtaacagtaaatgtatccttgttaattacagatgcaacagtcatatttccagatacgccagttgcacctgaattggaaaagttaacagcatctccaactttaagaccatgcttttctaaaacaattgtcgctgtagtagacccagaactaattgttatgcctccagttggccttctataataaggcattggacgtgaccaaagttctgctttcttcaatcgcatgttagataagtaacgggtttcgatcactgcatataagcgattaccacatacacccatactcatacaatgatagttaactttgtttgctgaagaggcattataatcaggatgaattggagttaaccattctggcatagaccaagtttgtccatcatctcctgatttaacccacgctacatggaggttttgtacaccatgacgttcgccagccataaatggtgcatatataacattgtcataaacaaacgtcttatcttgcgtccaagcattgtaaaatggtgtatccgtaatttttgtcagatttccatcgaagaaacccgcagatacgtaagtaaggggttgtccaacaatacgctcgtaaacaaaagcggcgtttttaaacttgcttatatctggtagtgttgtaaccttgaaagtgagattacgcccatcaacaatccatccatcaggcatagcagcaagaagctgtgttatggcattagtatcgtctgttgctccgtcacctactgctccccatcctctaggatcaccttcatctctccaacgtgacatttgaagttctggatacttggtagcaccatctgtatctttcaactgggtggctaaaaccaccccagtgtcagtaagaaagttggaaactgtgcttttaacagtcttatcaggttggtctaaaaccaggtaatcagaatcattaacttttgatgtttcgggtaaattagtaattgtagttgacatttttactcctatatataaaatttttgtgttgttactaaatcttctccagatttagtagctaatgtaattccacttatggtagcaatctcaacatttgcttgtttaagattgatatcttgcaaataacaattcaaaggcacattacccatatagaaaacttgggattgagagttaactaccgtaatggaatcgtgcattatttgaggataggcccatatatattgtgtaagtccgttaaacttgttggcgttcaacactatattacctataatcattttccaaagatacgtgcggtagtttgattcaggatcgaagccatacaaaggcgcaagagtagcatctggaacaaaacctgaaagatagtgaatatcagaatattcaaaacctaatttttcatgtggtatggtaattgcatcaaatgctttcatcatcttaccttccagaagccagtcgtaacactctgaacagtaaaaatcaaaagcttcaggaatgtagtaatcagacggaaagttaactttctccatcaaaccgttgccgataatcgatggtaagaacggaagcaatgttactttagctcctgggtaagatgctctagtatctgttgcaaactttgtaactcttaatcctagtttacgagacaaaaacgcataatattcgtcaaacggcgttcctgttttataattatctattgttcctgcatcttgtgcaaataagccagtttctgtgttgaatgccagtttggtagtgtaatcgtatatgcaaggcattttacttgctgtattataccaccaccaaggttcaccaatttgaatgacgactggaatattgttagcttgagaaatagatgctattttgttaataacattttgcaaatattccatgccttcgtcaatagttggagacaacacgtaactaggaggcttataccctgttgctgcaagagcatcgttccaatcatgctggacccattcaacaggacaaactgtgctcatcaactcaaaacttaaagagttcattattgtgtaacccttctcgttcgctatagaatggaaactatttagccaagcttctgtagctggattcaggaagacagatctatttaaagcccaagatgttccgttccatgagaactcataatagtgagacattccgttgtaatggttaatcattccacgataacctagtgcatatgtattataaataactctctctggagtcaggttatacatatcatcataagatgtgcacatgcgaatatcatgttcaggctggacaaggttgctgactttcatcatactataaccagaggtaggttcaagaagagtgaaagtaaaccaagcctcttgactgccaataggtacaacctcttctatttcttcataccttgctgtaatcaaagagaagaaaatattatctacagcactcatatccaaaggaatatcattattagggccagaaaagatattaccaaaatctaactttattgttgctgttttaccatcttctgaaatatctgaagcgtagaatcctaaactgacataatgaacacttttatccaggagagttacagtcaaagtcggcaccaaaggttcttctttaaaacctggcatgttttgactaatttccattttaaaccaaagtttctctgaacttaaatctgtgtgaggcaaatacccaacacctttatggtcatatttgtcaactgtactccagataacccctgccaaacctgctgttgttctgtttacaaatcttgcagtaaaagagtctccatgagatacaatagaacaactttccgttgcttgagaatctattgtccaatacataggagagaacattttaacgtcttttgtagtccaagttattggagtagtattttcttggatagtagttataatacaattgatggttgtttcaaccatcatgttattttgactaatccctgaaacttggagggagaagttaagatctattgagttctttggtcgttcaaatacttcaaccacaatagtagcagaatcagcatcatcagggaacacaagtgttcccgattttgcaatataatctctaccagcgactgctgttccgtcaacagtcgtgtagttaaatgaactcgcatttactctcttgtctcgcgtgacagtaaatatagcttgtttcattttaccccttactcggtaacgtccacactaatgtttgccattgtcaaaacagtaagtgcaggaatattgctcgaatccaaaacaggtaatgcattttggggattaagttttttgtataaagcatacaacccaaaaccacgtaacaactctccagcgtggaagccaaagaagactccgttatcactgtctggtctaggagcagcactccaacaaccattcataacgtgattaggaccaagaacaatatagttttcctgaataagttcaaaacagcggtctgctactattttaatgttagggatttctgaatcatatccagccatccccattatagcacatcccgccataaagagcgcagacatgtgacttgtaaagtcatctacaggcgcagccacttctccgttgggataaaatattgttggtgttcttccttcgttgttatcttggaaccacttcaggtagttgatccagtttttacaaactgttaaaagtttttcaggaacttcggctccttgtcgctttagttcgtataccgtatgacacgcacagaagaatgctcgtgcttcataaccatcccaagctttatcgttccaatgatacattgtgaactcatttttgtttccatatttatgagcatcccaacgatcccaaacataagcttgtgccattggcccaggcattacaggggcaaacttattataaaaccacatttgtgaatcatataaaaagtctatactgttgttcatacgaacccagtcaatatctctccctttaaaacaatataaggatgggtattgataacctgggtaaggaataccacgccaaccgtcatataatgctgtgttaggatcactaatattggaaaatggaatgattcctggagtataattcaaactgtcaagtttgtagtccagtattgtacaatctccaagttttgctgtataaggattttcagatgaaactgtaaccctgaaatacattgtgtaatcatcaccagcggctgaagaatacaaagatgggatatcgttaacacaataaaaatcgattgcccctgtcaagccatttgcaggttcagaatccaatgcaatgttgatctgatcaacagcagtcaggtttattgacgaaggttttgtctctgtgtcgttatgattgggttgccaagtgcttaatttgaaatcagcaggacttaaagctttagtaacccatgtagaatttgttttttctaacatggcccaccatctccaacctgcggaatccgtaatagtgatgttaaaatcatctgcgtatgaacgatatgtaatagattttaaattggctgttttagttggtgttaaccaaaacccaacagtaaaaccaccatctgaatccatattagcagaaactatctgatcactatttgtcccagcaataccagttgcatatttatattgtatcacagtattgcttccccagtcaacaatgattctaggttcagcaataatatatacaccgcctccaggaggttcagtgcgcataaaactggataaaggtatatccatcttagtcatattagctgcaccaagaggtaagccacaacgatatgtaactgtatctgtttcactcttagttttgcttaattctaactctggacggaaaagaacaccttcccctgcatcatctacaccttgaaactcaatacgaagttttgaattctgatccactctgaaccaaactgcctgttgttctagagttgtttgtgctgaaacatcttggcgaattactgtgtatcctaattcgtcacgagaatatttaggtattgctgtagatgggtatgagtaatcatacgagataccatctgtaaaaggaattattgctgtcgtagatctacggaagaatttatcaaacatatcgatgtttgagtagtcttgacacaacaataaagaagattgccagcaaagccagtattttctctctcctgtgatctgatacatcaagtagtttgcatcacaccaccacgtttcagcatcggaagcattatcttgatagcctaactctaccggaacgtttacaggacggttatgccacatctcattacgcttaagcagatatccaccgtgttctacaggattgcatgtagcgtagttgaatttatagtccccgttcacagaagcatttttaagttgtactgttccatattctgcaaagctatgaccaatagataaaatgttaccatcccaatcaactttacgtcctagacggtctataatccattcaacgtcccactgatctccatattctttccagttggttgaaccgtctgggttagttgcatacactgttgcgttaacagagttccaaccaagattaccattgaaagcaaaccaagctttatctaagtattctccccagtgtggcgcacctttaggaataacagtccttccgttaatccaagtcatcatgctacctttgaacccaccgtgcgtaggatacccatcatctgtgagagggtaatgtgctaaacgtggttctttaccattgatgatccagttgggacgatatttagccggaggatcaggaataggttggcccccgaagaagtgatctatgtaggcttgccaatacttctcggcagattcaagatattttttatcccctgtagccaaatatgcataagcaaaaccagtgataagcacagattgaccttcggtagtagcgtcaccgtttggttgcccttcctgcgctgtccagttaataaagtgtctgttgttcgccaatacgttttgtgggttgataatgtaatgctgaaccgtaggatctatggtaagcccagtatttctttccaaaaactgaaccaacccttcaatcaactgaaacgtgttgtcaatattgtgcttaactcttgacattaaataactcctgctaaaactaagctacctgtccaagttgctccattatcataactgataaactcaaaagtgtctgttgcaccttgacgataactcaaaactggggaacgaccaaaactccattttacagaagaaggccatacgactttatttgcaccacttccttgacgcaaagctaaagtaaagtttaaaactgcatttgttgctactggaatattagtaaaactaattgtgcacgttgcagaagataaagttgctgaaaaatattctgccgaactaatatctaatgccaaggtgctagttactgttccacgtgcatttaaacggtttctagttacccctgcaccactcaacagcactttcaaagaagttttctttgttattccagcctgatctgttacaaagaaatctgtatcagacacagaggttgtttgtgtaagatcagaaattttgatcgtagttaaatctgccattactcttctactcctgtcatagctgttataccgaaattattgtttgtagctaatgcatcacctttttcgttaaccatgattctttccactgctgactctgtagaccattcattccaccaagtagagtaggtatcacgtggcaaaccaacaaagttgataaagcgataaagtctattcgaattctcaacaatactgtaagcttcacaaatgatcgctgcattgtcttcaccagcttcagggtaaataaactcttcagaaggttgagtattgaatatagatgttactgtatctgctccaactgtgaccataactgtcattgagtaataacgttgatccatatcataagcagatttataactgtttattgtatctatgtaaacgtttttggcaatctctgctaaaaatattctgtctacaatatctttctttctggcaacaccaataatctcttgtaagtaaggaacaccgaagttagtatttaaaaaccactccccacgatacgtgttcagagttatcccaagagtttgtgctgcaatttctgttctggttgtagtgaggaccaaatccccatcatccatgatgaggtcgttgtttggtccaagctttaagtcaaaagggattcccatgttaggctcctgttggttttaaggactcggcacttgagctaccgctctgaatgttacgaaccgtgtgcgtgtgagtatggaatccgatgccattaccatctgtcatcaaaccgttctgatctattttaagaccattgatatctgccacgccgcctttgaaagttaaattaccaccttttagatccaaagtccctgtgccatctgtaatagtaataccggaggtagtaatatctattttcatcccttgactattccatattgaaatattacctgtagggtcgaaagaagcgtacgtgtttttgttattagaaataactaccttttcagaatctacagcttctgttgaatctgttcctgtagctattttgggaataaaacaaatagggtataatccacaaggctgcataaaagtggatgatgaagaggtttctccactagtttgcagtgcattagaaggatctctttcagaaaatataacagttcctaaatcgccagctttaatagggaatgagattcttcctgtcccacccccattaaccataaaaggaacatcttcaaaagtagggtaatctatttcatcttcactagatcgtgtgtttttaacaatagattctaaagtgacagttttagcaccataattaacgccagtcacacggactggcagggctgtatggacttctgctaattgttctgtcattaagccacgcattgctgtgacaaaatttaatgcatccattatttctccttagtcattatttcagacacggatacacactccacttcagtaacccactctttaccctctttatcaccattgtgagaaacagaaacaactttaaatggaccatcataatctcggctttttaaccagattgttttttctggtaatattgcaccattcatttggcatttgaatttaacaccatctgtaggtttctttgccttcttagatgacttggtaacttttttagtaggttttatatcgttgtggaatggttctggacttcctatcagaccagtttcagggctaatgtaggcgctacgtgcactagacatttgtgaacgaggagtgacataaacagatccatcttgtattgacacattgtgatcaatacttttacaaatatgttctaggtaatgtgaagtgtttccacacatagctgttgctgaggataaggtttgatcattatctacgtgtatattaccttctgtagttcctaaatcctttaccaaatctgtcacaacacgtttgattttagttccttttggataactgcggcttgttcttgcttctgcaatgttaataccgccatctaaacaatgtaaagtggttttcctgtctaccgagtcaaaagtatcactaacccactggatagttcctttgaagaccataaccaactcttcaccttcgtatcctaccgctaaagcaacagccaagttatttcggataccacggtttatatagttaacagtatcatccgaaaggttataaactacaatctctgcttggttaggatctttgttattatctttcttaatactgaaggttatttgatggtcttgcaacaaatacaaattagaagaggatttttctgttgttcctaacttggtatccaaatctacggtagatttaaatgccgagttcttagttctcttctgtattgtgtttgtggcagaaccaattaataatttgtatgccctgtttaaattaggggcaggagcttttgtatactctctttctgccatattatctccttacgcctgattaggtttgttatcaatataaaccaatacaaaacgtttatctcgacctgtagttgtatacccaggacgaccattaatcttaactgtatctactatatacaattgaccatctggcacactttcaagatatttataaggtttaagaagatctaaaccatttgtaactttgaaacttatacttggatcatctccagataatccaatctgacattgccaagactcgtccctagtgttccaccttaaacgaatgtcataatcaactccatccaatgtaactgtatatgtttgatcaggatactcttcagttttcagataccatgcaaaataagtagaatggtcctctagataaactgcactgtagcctgtattacttgtagaccttgttgttttagaataagttgtcactattaacctccgttgtttgttggggtattagtagcttcagcagttcttactgtaatatctgacttaggaccattcgaagcaacttgacctttcacatcaccaccagtcgatttatttggtgctgtttttacagcaatagcattagtagtggccttagaaacgagacggatttgttcaaactctaaatcaaattgcatagcaaattcagatttataatcataagagtagcctttgagaataacattttcaaaagtcctgtgttctgtcaaaagaactattggttgtcttgactcaaatatttgatctagatattctattgcttgagaaatccttgccccatttacacctgcactgtataattgatctcgcataagccgaattggtgtttcggtaattatacctgaaagtttaaatctgttgttttttatttgaacgtggtcacttacatccgatcctgattctactggaaaacttgtaacatctgcctcacgagttacacctacttcagggaccgaatcgaaagtaattgctttaaagttttgaatgtattgatcacttgaactattataaacacttgctaaaatgcagaaaccattatcaccctttacagtagatgccgaggtatttttattctcggtagagttgatttcctgttctgccttatagacggctctcaccgactgttgtggagtcattgctgccatgataacctcgtagtaatgataatgccaaaaggaagatgcaagatcttcccatattgattattgtaacacatatagaaacttcgtgtcaagggaaacgaaaaaaccccgccgaagcgaggtattttattaaccaccagtgttagattgtccacttagagttgaaagagacaaacgttggaaaatgctatttccgctattttgaattcttgcatcaagaatatttccaaactctgaagaagggctgatcgtaactttgataagatcacttggtattgctaccgtattctcaacaacaggttgattacgagctaaacttacgttaggtgtataggctgattcacgtaaatcttcaagaggattaccaaatccaaaagcacgtgtccttgtttgttctcctctccacatacgaccaatatcttgtgtatcaatacctgttaaattgtacaaccaatctgctacaggattgctgtctccactttgcgttgcctcgtatgctttttgtgccatagatttattaggatctgtgttatctccgaatgtatctgggaatgtttctttaagccaattggtaaaagatccgaaaccatctcccaattctttagcaaaacctcctaattgaactgttgtcttaccgagttcttcacctaaatctttggcaaactgatctagcactttaacattatccaaccaagaatcatctttaggtaaggctttcacaaaaccctctaagaagtggttctgcataccttgacctacaagctgaactgttgcagcaaaacgagtaaactctttagaagcatctatttgttcttgagttaatgttgcacctgttctttgaagcattttcaatacttcattatacccttcgccgtttttactaaacatacgttggtagtgcataaggtcatcaccaaggttttcaagaagtcgcccgatttgagcagagttcatacctcgacgctcaccttcattaacagtagcctgaataaaagctaaaggatcgttctgtaaaggtttaagctgttctggattccatccaaacaagttcataatgtcatttatgccattatcaccgcctttccacttcccttctttctcattccattttgaggctaaaactgtattaccaagacgttcacgaacgtctttaatattatctattgacttaataatatttgcagagtctacaccgttctgttcaccccaagctgtcattgccaaaatagcgttggcgtttgtctgaacgttttcagcaccacgacttacaagtttaattctttcgttagtattcatcaacgtgttaccaactacagaaccacctgcaaacagcccagcacctattaaagaggatgcaatcatgcgtggattaagacctgctgcaccttcttttaattgtccatatcgacgatcacgatcacgtttttgttgagctaattcacgcttacggttgcgttctatatctctaagttcacgatttcgcatacgtatacgcattttagcttctgcttctagctctctacgaatacggcgacgaccgcgaacctcgtcttctatttcagcattcttttcacgatgagatctgcgtaaggcatctaactggtgagcaagagcttggttcatacgttgaagagttatttcaccctttttgtattgctctacagctttacccataacgtctctagcttcttgcatagcgccaggactggctttactaaatgctcttgctctgtagttaaagttctgcaaacgaagattacctaaatcttccctacgtgctgctgctttagcttcgcgttcagcagctttacgagctttttcttcttctcttccttgcggggttttaggagttctaggtctaccagatccgcctacaggagcacccgctccagcgggagtaggtgctcgcataggtttttgactttgagtttgagctttcttaagttcacgctcaatctctttcatttgttgtctaaacttggtcatgttcatagtgggttgcattttatttgcaaacttctgaacactcaacacggcatcttttgcttccttgagactgttcttgtccaccttaaacgtaaccacgttactaattttggaggcgttaatttctaacaaagtatgttcctcgtgttggattaaacaagtttcttaaacaaatctgacattttatttgtctggttttgatgttttctctttattgaagtgtctttaacatacatggcttcaacatactcttcgtatgttaaatactcatatagttttaaaagataatctacaccgtagttatccaatgttttcagtgtttccgaggtatctttaacgactttgcaaagaataacctcataccacttcaaagtaagggttttcgttgctgtgtcgatagcatgttcaatcgcaggtgttaatttgctgctaaactcgtcatcgcctgaagctgtgtgaacagttttccgaaacctcgtttgaaaaaagggccgtagtttacctccagaactttagccgtaagatctaacactagttctgggtgagccataaataattcatcaaaattttctgcaacgttataaccgtcttgagtataaacctcatctaaaattttacgcaaaaagacagttaaattcttttcttccaaacctgcaaaaagttggatcagtgccatagcaatacgtgactcaaggtcttcgtctgcggacattgttgctgtgctatacatagtcataggaacaaccagaacactacccaattctggaatcctgtcaaacactttagatggactccacttcataatacggaaagtaacagatctgtttccatctaacggaatctcaactacttcttctggtcgagcatatttttgaattgtgttatcttgttcttgtgacatattttcctctctaatgataaaaagggcatttctgccctttatttattattgaattgtcagagcagagattgagttaagggtgctcaaacctacaccaacttcattacgcagaaggcttgcatctttaagaccaataacccattggttctctgtaatcatttcgccaatagtatcttcaggctgactttgaatccatccaatcgtgctaatgtaatagccacgtgggtcttccaagtaaacactgaatgctacttgacgagtagaatacattgtttgtgcatatgttgcaagtacttcgttagctggagatgtgcgttgcaaagacattgtgagtgttcccaaacggttacgactcaaagccagtgaaacgtcgccatcagtccctgaataagggttgataatatcgttagatttagcaataacgattttagtatcagccgcaaaaccccacggttcccaaccacctaatgaaaggataacttgagatgggtcgtaggccattacgccagtaagcattttatccattatagatcctctttattttgataacgggggcttaacccccgccgtattagtttgcagtacggttagtcaggacgttgacctgaactttaacgtaatggaagaagccagcgtaaacatattcaatcttcatcccatcaaccagacgagcattgatttgcgcatcagtcatatcggcacgagaaggaatataaacaataggtttcagatcgacacgcaaaccagtctcttcagaagtaacaacttcattagcgatggaaccgttgaggatacctacttggatcgggttagccagaacagcttgtcgaatcaaagccagaccgtttgaagtagcacgaacaccaccacccagcatagactgttgtttaaacagtgtaaacagtgattcttctgtgcgagctttaaaccacaaagcatgaacaacatagtcaacaaagaaaccgctaaccatcaatccgtcacgatagaacagagttccatgctctttacgatagatgttaccgttctgttgaacaatattgttttccgaagtctgatccagtttctcgatctctacacctaccagtgttttaccgtgaagagtagttaaaccaggttgacgagaacagattgcaccaacaactgctgcttctgggaatacagaatctgcatcggtcatccacaaagcaaagcaagtgttattataaccgaggtcattcagtcgttccagaacgttatcttctgctttcgttttaattccaggttcagaactagagaagaagtaaactttatcattctcttctacccatgcagccaaagctagaacatcttctgtttcatgggaatctgaaagggtccagaaccagctactatcaatatcatgcagagcagcaatatcttccgtaatagcattagcatagacagtttcttgagtgaaacctgtgctgtcaatagaaccaaacttaccttctactggagtgaaagtcattgcagcgccgttgttagctactgtaaacagtgggccataaacaagatctgcattgatcatttcagtcagaccaactacaatctcttgtgcagttgcagtatcatcagcaatgaatttaaattcttttttcttggtaccatttttgatggtaattgtgtaagttgcactatcttctacgattggagttagagtaaagtttgtaacttcacgcttaccaaccataacttgatctggtgggaacaaaccgttaaacagtttgactgccataatatacgctggatcatttaatccataacctgcttccaaaagtgcatcggaagaagagtaatattgaatcgcttcgccagttacgttctgacctgtaaggatcaggggaatatcgaactgggtttcacgaattgatgtagcaccatacagtaaagttacggttgctaccttttcacgaaaagttgccatttatgctcctgttagaaacttaattccaatgagtttattcggtagggttgttaaataatgatccattttgattgactgacgagtattcgcctggaaccaaagtaattgcataaaccctgtttaaaattaaatctaaagggataggatcattgtaactatctctgaatgaacggatgtgcatgttgattgcttctattggtgtagtagcaatatcttcaatactcatacgagtgttgaacgtcataaccatacgcgctcttaattcagtcttatctccgtctacaattgtattggcttgagctatattagaggctcctaaaaaaccaagacctttagaatagagatgtttgtattttaattcctctgcaccacggagagcagccagaacatagctcatcaaagtggtaggtcttccacgataggctacaagttcaatctcgtaggtattatccatctcgtaggaatacatatcattttgaatagagcttcggtaagaccaacctccagaatcgctggaagtgataatacgtagtgctaggtaaggagtatccggtttaatgaatggcttatccataacatagactggcaaccccactaattcttgaaggaagccacctatatttaagtagatggcattttcgtatgcttctatttgttggtaaatattagccaaagtttcctccttctgttccataagaccaggagttgttttgatcgtctaatattgcaacacagtgatgatggactaagaaagatgttctggtccaatctttaagggaataaacagaataccatttaccatctatcagtatttgatctgataactgttctgttccttgttgtaaccccttcatttctgttttagtccagaaactatattgagccttagatctgtaaccttcaggtagtgcattcagtgttttaccagatatcggttgtaagcttgccgcttcaatggtaaatggttcatattctacactactttctaagttagcaataactgaaccaactgcctctttgtagactcgtctacgaccttggtattcagttagatcaaggagagtaaattgatctagtaacacttgtatctcctttttagtatacatcaaaaacaaagtttatgcaagttattttttagacttgctttttgtgatttttgtttcgattgcatttatcatgtgtgccgtatctattaagatatgattagaatagttgcctttatcttgtcgtatcttaattgtcacaggagataaaggacgataacgttgcattgcaatcgcttgagctattccttctttagaggctctcgctataggctcaaaaacagctaacccgacattattcagtaagtaatttgcaaacacctgcttcatctttttagcgatattgttctgtgagataacagcaccatctgttataaaagggcgagcaggtatgttatttgtttcattaccatattgttgaatagctgctattgcaggaatagtgagatctgattcaggatgccttttgtttgttaaaaaaccagcctcgatttgaacttgttcaaggtccatcatctttttgataatggtttcccaatctgtatcgtcacctggtaagatctcactttccattatttgtccctacgataaaaagaaacaggtccgtaatttcgcaaataattaacgttgcgtgtattacgatcaacacttgcaacattaccaagaccatttacagagttaggatcgctgtttactcgatcaatctcacgagcatccacaccgccaataagaactttgctggttgctcctcgtcctgccgtgcacccagggatcaccataagaccatttaaatagttatgatagatatcttcccacggagatttataagtgacttgaccgttgttaaatttaacttcaacttcgacttcacccatcttttctctgcgggacatagctcctcctgtttgagtactgttgttaagaacatcgttgttccataaatactctaaaacagaaatcaagctgttccacaatactgcacacttatccttatcttcattccctataatatcaatccaagtttggatgatctgaatcagcatcgcatctggcaatacttcttgagaagggttataaacaagaattcggatcagtggtgccaactctaccgcattcagcataagcccaccttattttttacgaagggaacgttttttagctggagtgtctaccgcttctacttttaaaacttcttctttgatttttacaggcgaatagtcttcgtaacttacaagatagtaacccatgtgttcttcacttgcaaaaggatgcacaagaatacctgtttcttctctcaaacgcgtgaggaagtgaatgaagtttccacctaatgcaatacgaccatctttcagatagaaagacattggtggcgcagatactaatgttttatcagcatcgatcagtggtgaaagcgattcgaaatcacgaatcatttcgtgataactaaagtaacgtttcaattctgggtttgacatatcaatctcctcattgtaaactcttattgtaaacagaaaaggctcgcccatcacggacgagcctctaatattaacagagttctattaagccgtagtcagtttaactaccagttctggacgggtgttaactgcgagcagcgagaattcagactgaagctgaatagtgcggtgctggctgatcatgaaggtatacagttcacgagcctgggtgttagccagttctggagtatccgcaggagcgtagtgcgcctggaacataccttcgatacccataggcagaacatatgcttcaccagcagggatgttaccgtggatatcttcaatgtaagttacaccgttccattcgaacacctgaacatccatgttaccgctaaggcggtcacgcagaaggttaggagttccctggaaatactgatatgcctggcgaacaaatggagagttaaccagagatgcgaaaaactcacgagaacacagagctacgatacgggtagcggtagagccatcttgtttagtatcgatgatgtaagcacgagcttcttgttcgatagtggctgctggagaaacagtggtagaagcaaagtctacagcaacagatttctgggtagcaccccattcggtataccagttgtatgcagagttagctgcaccacctacgccgttataggcacgacccataactgcttctgcaaagattttttctttggtttttgctacgtcacgcagaatctgggacatatagcggttaacaacttcagcgtctgtgcgcagagagtcattgattcctgccattgcaaagctacggaaagattgaatatcttgagctttgatgtttttatccagagggaagaacggaatgcggaacgctttcagttctggatttttcatggtcaggaagttacgctcaccaccacgctcacgagcggagatcagagcatcttcagctttttctttaccgatttcaatagcggtagttgaatgatagtcaacactgaacagattcaggctggcaatcagtgtatcaggggaatcctgaagttcgatcatagggctgaaatctacaacctgatctaaaattactgctggcattatattaatctcctattaaaattaagaagtgaaaacgactttttcggtcagtttcagatcatgtgtttccagtgctgctacgccagcgtcatctaccagggagccatctttgtagtatactgcaaagcggttcagagtaagatcacgtacgccaacaactgcggtgaaagtgtcacctactgctacatcgtcaataccgaacagagcatcagtccaaatcagaactttagcagcatcggcagcttcagctttagcagcttcagtgttgtcagctttcaggatagaaccaattttcatggttgctgttttaaccaggttaacttcacgtacacagtgtccaaggtcagagctaaacactccacccagaaccaggtgtccgaattcctggcgagagttagataaagtaattcctttagccatttaatttaatctcctaagattattgtttctgggcttttttaagttcagcagctttctgactaatcagttcagaagaagatttagctacaggttgagtattgatcacgtcaacaccacgttctgttccaaattctttcttaactgcgtccacttctgcacgtgctttttcgaatgctttgacgatagtttcagcgatatctttattgtctaccagatatttaacgagagcttcaactttgtcttcttctacgaaagaataggatttaacaacagttgtatattcttcttcgatacgagcttgttcagctttacgaagttcttctgcttcctgttctgccttagcgattttttcctgtgcttcaagttctgcggctgcacgtgcatctgcggctgctttttctacagcttcagcgatctgtgctttcatcagttcttggaattcagcagatttcaggaattcttctttgttgatattttccaaaggggtttctcctttattgattccagttgtgtttgcagatttatcacctgcggggttagcctctgcatccacagagtttgtattagctttagcgtgagctttcactaaataatctttaacttgactttgtttcaaggaactcttaacgagattactcaaagcatcgtctaaaacgtcctcaaagaaatcaatggagatcattacatcaccatcaacaacttgataatcagtaacacgaacaacaggtttagcaacctcttcaacttcaacattcagtccgtcaactttaaagccaacagagaacataccgttatctgaacagaaaattgcaacgttcgcatcgaagtcagcatcaataacgtaaacccaatcccaaccagcttcaaatttttcacgcactttttcagaaatgcgatccatcacttgttgaatatagcttttacgtaactctgtaacatcttctcccattttagtgagagcttctaacttttcaaaagttatgtctggagatccgtctgacttaaataaaaggggggtattataaccagatgccgctccaccttgaaaatcaaaagtgtaagcaatatgagcacctagtgactcttccgtagcttttccagaaaagtctacgttagttaaatatgtatttgccacgattagtccttcttgactttaacgcccattgcgcctatactcaaacctttaagaacgccgtctttacgagcttgatataattcagggtcgttgaattggattttaacgaggggagttccttccacaacgagttcgtcaccaatgtaacaatcgacttcatttacccaagctttaattggagaaaacttatcggtgttcttcatgtgaccaagattaccactaatgttggtaatattctcattgaagttgtcaaccatcttcctgatttccaaatcagacatgcgctctccgtggagatcgtcttcttcaggtggacaatacaagacctcaataacctgcatcttttcttcatcaaatttattttgtttgactagagtttcaggtttttgagatggtatagaatcttgttgggattcagaattctctttaaaaccagcgaaggttttgaagagttcccacagtgtatctttattgagttccatttgaaacctcttaaaaacagatatcccataaatcttagtctacaggatatttgttgttttgtcaatagaaaaattaaaaaatattaaatatttttaattcgacaggttttgtgctgaaggatctagagcagaaacagtatcagacgttccagttcccgctgtagctaaaccatctcctgatcgtgaagttttgtcaggcatatagttatccatgaattcaaagaatttctctttattggcaacaatatcatcagggatgcgatattcaaaaccaattttttcaagaaactcgttaataacttctggggttaaaggtatcgcaccagaggctaccaactgctgaatagctttagcatttacttcaatatcaggatcacccatttcaccactgacaaacttaggcatatcttcatcagaaaggaagatattgtttagtcgtagtgtttgagggatcagttgagagtttatagcttctacaacaatatctacatctcgctcaataaaatgagacaatagtgtttgtttgttgtcagaaagcgcaaaacttcctgcatcaccatcacccataatcaacacacccgcaccaaagcggttataaatatccagtttacgttgtttgattagtgcttgagtgtcaaactgtttacccgtgccatcaacaccttggaatacaagatcgtattgacgcatcactgtgccttcataaacgtctgaaggcaacaccatgtaggcttgctcaccagagtgcaggttagcgatgttggcttggagaactcgaagagattgtgcttcatcccccgcaggatctaaagatgccctgtttagaatgtcacttggaaccttaagaactggcataccgcctaaatctttagataccccaataacctcaaattcctgaataagagatttttcacgccagctacgatatacagctaacagcggactaactcctaatgggttattctctgtaatgttgctaccaacaaataaaactttactgaaaggaatttctttaatacccgtttctaaagttgtattacctggagctaacatcgtattggtgatattctggttaacagatagaatagaacgtccgtctgccgaatatgtaaaaggttgagccatgtgaagagtgtcttgtggtctaaaagccaatttggtcaatcgccaatacccaaagtaaggacttttaggatcttcatccaactcataaactttctctgcgaatgcaaacccgtattgcttatatgtcaaaagtgatctaacatattgacgtagaggggcttgcatattgcgtaacatgtaatcaataaacttagcagcttttcttgactttgccgagtttacattgtattttatcttgaaatcaaagaatgctctatctacaaagatatatgtagcgtcaagagcagtagctacatcagggtcttcacccatctctttaaaagtgcgaatacattctgggaaacgagtctcatagggttgcatgaaattcgtaaatgctttgattgagtttaatccgatcgttcctatttcaggtgacaggagtaacgttttctctttctctgcttcttttgttggagagagagcagctttatttgcagcacctttgtcccagtattctgactttttagtgtacgttctttttcctgttgacacttgttcctccagtcaggtataaaatcgttaaggcaaggctgtattatatacaacctcacctatctagttataatacactttaaagtgacttttgtcaacaattatcttttacggacactatacggttggtgaaatccgctgttcattctactgtatcctgtaggagcatttgttgttgtagggatagctactgccttgaaagtattctcttttgacagagtattaaaacaagaggcagcagcatcgggaatgtcatctttaatagtggctgtagatctttcaccattgaaagattccaattctttataaaagtgggttaaagtttccacattagggaaagaatcttcaacaatataaacaaaaccgttttgacacgcactactaaaaggcataaaacgctgaagctttttcttgttgcctggcatagcatctggtttaacaacaaaaccctctagaataagtttctttgcagactctgtatattctattgtcccagcaccagatgggtctttaggaagaactactgtaacttctggtccatcaatcttacattgatttagaatcaggttatccctttccccaggtagtcttcgaaaccttccgaaaattttacttttctcatccacagcatcataatcatagtcgccaaagatgtagatattaccatcttggtctttacccattttgacagatgttgtatagtctggataacggtaagtttctgtaggaactgaaacagctttatcccatgctcgcattagtttaagattgttaggaacatctttataagatattttatgtaaccattcacgttgaaagaaagattctttctgaatacgtgcaaaccaacaaccgtccagaagctgtgctcttttaatcgctgtttgtgatttaagctgtgctaagtaattagggttaagcctaatgagttctgggttatcaaatattgtcccaccaataaatacgaaagtttgtggttcaataacaactgtctcccctgtattagggttttccatgaaacagttttctggatatttttctttcagttcttcaggtgtgtcagcaaaaacaggttcatcgttaacaatgatgaaatatttttgtttaccacacatatcttctctaggataaccaccctcatcaagataaggtaacacccaattcaaaacccaactatctgcgtcagggttacatgtgcacaacatgaaacttgtttctgactctgaacgcatacgtcccagaagatagagcatttcatattgtgtgaagtgggttaactcgtcaaaacctaccattgaatactgtgtaccctgatggtttgtttctgctgttacgttatcattttccaaatggtcaaatttgagggttccaccaccagtggcttcaaaaatgatatccattgtactttcttttacgtgtggttgtaaaggaagatacaaacgttttgcttctgtaaacagaccacctggtttacggtgagcagtggtgttcttacggaaaagtattccaccaaacaaagggtctttgtaggcgtattttaaagctttcagcaatagcagacgagacttgccgctattcccagtaacgaagcagtgatcgccacgtctaaagacaatatagctgcttggagtctcaaaacaatatttcttatcatctgttgaaataacctcgtgtatttttaagtaagtgctctttttaccaggtatgttaaaaggaacacgtttaccgaatggattttctacataaccttgcaggggttttcctatttgctgtaatgcaataagatctcctacaagaatctcgaaatgtttctttgatttctttttcttatagaatacaaaacggtgttccatactaagttcttggaacaatcctacaccatttgttattctgtataggttatctccatttaaatcatattctacaaaataaggtgttaccaattctaaacgcttattcttaggattgtattggtaaatatcctctcccgaatattcactaatctgcctccaaccatcttttgtaagatattccgtttcagcagaaacacatccagcggcaccaccataaatcataacttctggcattaaatccattgcaagagtctgtttacctggttgagcctttacttcaatatctactccagctaaactacctgctgcctttttctttgccacattccctccttattcagcaataatacctttttcttgcagtgttttaatatatgtttgtaaacctagaacctttcgtctatcttgctcccaaagagcattgttttgagtttggttgttaacaacttcagatctactaaccccttttgcaatttgatttttagtgtatggcacaggtgtcgatactttatccatcaagtccgaatctggacctttaaccttttctatggctacagccttacactcatcaataagaactggggaaacttttgttgatgatgaacaacccgtcatcattaggggcaaagcaagacatactgccagaattagtttttgcataattcgttgtatcctcattgatcgtttgtttcttcttctctaaatctgcttttaattgtgcattgtttttagacaagttgtttatttgagtatttaataatgctctttcttcatctgctcttttttgaagaacatcaaaatcttgttttgcttttgcttgggctgcttgatatacttgcgtttgataggcaacaccatctttgtagccagactcacgtatcttgttataagcaaacattccggcaagaataagaacaacaattaaaacaaaacttaatacagttttagtctttagattcattatcttctacttcctcataaaattttattttaggaagagaaggggcgttgttaactatcttcttcacttccattggttgagcctccgctaggatctcttcccaagtcttctctgtcatcataaagttcttcctccaagttccaagcccgtgtgtaatcattctttttatctttatgataagaagaacggttaaaatcatttttaactacaaaattgcgcatattttatcctcctcatatctgatcttatcacaaaatgatatataattgcaagcattaaaaataaaaaagcccctaacctttcggagaggggtgttaaaaataacaaggaggaggggcaaagccccaagagaggagagggattagattacgatctttttaggaacgtcatctgtattctgcacggcaggggtttcttcttccatagcttcataatgttcttctaaccattcttctacttgctctttaatagctttagctgcttggaagcggagaggttttgctgcatccttatcattaacaagttcaacaatataccgcatagcttctaaataatgctctgcaaataattcttttacacttgcttgcgccgcatccattgcgtaaatctgttgcatttcggctgttggtcttccacgaccacgttttagtttaggcataataaaatcctcctaaactttatcttaacatataaaaaataataagtcaagttttattcaatagatagcaaaaagcctccaataaggaggctaataaatactgttaagttgaaatgctttattataattgtgcgttaaggagtcgcacccctccatcttagaaggaaccaggcaaggtcttcaaagataccgctggcaagcgatatttggaggcgggaattggatttgaaccaatgatctccagcatatgaaactggcgaggacgaccagactcctctatcccgctattatgtatcccgtgctggattcgaaccagcgtcacaataaattgtggaactcttatcctgtaagtctaacgggaaactggttgtttcgacaagaatcgaacttgtatctcacgattatcagtcgtgggttctaccattgaactacgaaacatttaattggtcggaattgcaggattcgaacctgcgacaatacgctcccaaagcgtacactctaccaaactgagctaaattccgtcataacattctaaaggtacaccgcaaaagaaagtgttcggcggctcctcgtttttaacttcacacttgaggtagtgactcacctttgttatatggctggtctgtctggatttgaaccaaacctacgacagtcaaagtgtcgtgtgctaccggactacactacaaaccaataaatcatttaatgaagggcaaccagaagaggtgtgagagaaggagaaaaccatgagtaaaagagaaagttttctggcaaccctttattaaataatttggagcgttctacgaggatcgaactcgtgccgccgacttggaaggacggaatgctaccgctacaccaagaacgccctacatctttactatactacattttactattggttgtcaatagttttattgagtgggacagtgacgagtatcccgttgggtgactagccctacgttcggtaagctaacttaaaataatacaaaaccaaagtcaaatattattcttcgttgctgtcgatgggagagccgcttcttcttttgccccatccaaccctactccggtcaagtttcaggtaactctttttgtgtgatcaacacaatactaagaacactgtctttcgaacagaccttgtgcacaggatacgacgatcaatcacagtgcccttaatgttgtgttggtgctcccactctgaatcgaacagagatccacggattacaaaaccgttgtaatagccattatactataggagcaaagctgggactcggcaggattcgatacctgcaattccagaagcctcttggcgactgggggtttaccttaactctacaagtccataattctggcgatgagtataggatttgaacctatatcgttctttcgaacacggattaacagtccgccgcttaaaccattcagccaactcatcttaatttggtggtgagtgatggagtcgaaccacccgaatcgcaatgataacagatttacagtctgccccgctacccctacggaattaactcacctaaatactaatatttaaattctttttaaaatagccagtgttcacaccactaactatgagttatcggtaaagtggttaccgccacttgccgaagatttactctgctaaagtaagctatattaaaaagaacttggcggtagatgtaggactcgaacctacacgccgtttccgactacagtttagcaaactgtcgtggctaccatttcaccaatctacctaatctggcggaaggagggagagtcgaactcccaaggctctttcaagctcggcggttttcaagaccgttttcgtcgccacgtcgatttgcccttccatacttataatctaacataaaataatgttagttgtcaatagttttttaaaacattttaccatgaatggtgactccaccaaccccgttctccacgaacggtgctcttcgagcatttatgtcttctaatataaccgtggcctttgtttataatgtctgacatacttgcctcattctctgccaccatgatcagtagtgggttcaaatcacttgagctaattcatgttaaaatgttctggtgtctttacaaggaatcgaaccttgactaagagtttagaagactcctgtgctatccattacaccataaagacgttggtggccctggctggtttcgatccagctacctatcggttatgagccgacagctctcccattgagctacagggccataaatgtggtaacagagacgctgacgattgtagagaaccgctacattctctaagaccaagcccaactttcagcgtaaaagttcttgagccgagactattccttttcctctttggtagctaacctctgaagaagtagtctctaacactaccgcagtgcaagcccctcttaacaaaagtcaacgaccctgttaccacacttgatgaacgtatcctaacatatgaaggatacgtttgtcaatagttatttaagcatattctgctacttcttcactatctttcttttccagaagagcaacaaccaaagcttctgcgttaaaagtattaatttcaagatccatctctcgcatcagagccaagaaatcaataactttaacatctttagtgattacaatagaagttgtaccattgttttcagatgtgtatttttctggatggaaaagaaaaactgcattgccgttaccatcttcaaacacgtgtttttctaatagagacattgttcctcctaatctgttagttgttcgtcctctaccaagtttgacacgagaacgtctgttatacattgatgctgataaaccctttcgggactatcatcaaaagccaaacctgtgcaaatagaacagaggtctgattccttgatgactccatttttatcacgtctggagtggtttgtcaaatgcctattacaagcacggcacctaaaataaaagctattttgcatatatccttcgaaccccaaatggggagtgatcggcatctcctcacttctagtttagcacgaaggatatcttaagtcaactaggaataattttatcctttttggtttcataaccagtccaatctaaatcctcccagccttcatcatcgtctaacacattgtcatcatcataagatgcaatgttataaatagaacgtggattacaagacatacaatctttaataggcacatgatgccatttgcacggattctcactaatcattgcacacctccattcttgttaagatgtttcgtactgtacttcatactaatctatagatcaagtattatttataataatttattattttatttttaattgatagaagttgatctgtacgtaccagttctgatacactgtacctgtttgttagatctaataattatttattattaatatttaataattattaatctagtacggagtacagtacttgagtacagagtactacatcaccaacctaaaaccttaactcgctgcgctcgttaatagatttctagggtagcatacttcgaacacaaaagtcaagagttttattaaaaattattttctgtgtgcaaaaaccttgcttaaacaggtagcaatagtatagcctataacataagcttcataagaggctgtagagcaagtttacttactaggtaaggtgatttatcatcttatatataaaaagtgcgtcagagacgcgtttaggaggctagaatgagatttacaagacaagaggtagttgtacttgacgaagcaatgacaatgatcgtcttttactatgaggatatcttctgtgcagcatatcatttaaatgatattcagattgaatggaagcagatagcattcctgttggacaagagtaatcatttggatggctttgagtatttggagtgggatggtgcaaaggaagttaaaattgtagggttaaatcacgatcagattgttgacatgcaatgtaagttgtgtcattatcagttctcaaacaaagtcaaactcgaaaggcaattggaggtggttaaagatgatagcactaattagtgagaacccaatcctggcaacaattgttctgatttttgctatgtgtgtggttcacgatgttctggtaagctactttgacaacaaaggaggtaagaaatgaaagacgttattcaaatggtattccgtgtaacactagatggtatacacgttgttaaaggcacacagaactacacagaagcgaaagaacacttcttagagtggattgatacattgaagacacaagatgtgtcctacagccgtgttacgcttactggcgaccataccccaatcatggagtacgtaaagaaatgagcaactatccaattacaggcgtaacctattcttgtgtagcaaaaggtggtttatatacagtagtggaatttccaaaacctgccggaatatctaaagtgtttgatcctgtggttatctatgaagatcttagaacaggtcaacgttactggagatttgtgaatgactttttagatcgtatggaggtagtaagtggcgaaaacaccgaagaagttatcaaagccaaagaacgttactaagaaaccgaaaaacgaggttctcaaagtagccaaatcagttcctaagaaagataaatttgatacggaggcattcgtctttgacacaccaatcgagaagattgaagaagcgttgcaacgtcttgaggagcaggaggcagatccaatggctgaaccactcacggcagaccaaatttattggtggggaatatggactttgcaactaccagaagcaaaaagaagagggatgacttcagttcctacttgtgcaaaagaagcggctgtgttaggtttgtatgagaaatctaaaaagaggagcaaaaagaaatgatcaagactttcctgaagtgtgtaaaagcaacagataatgagtggtactctggattattcactgctggcaagaaatacgagatcctcaacgggacaggcaagaagttttctctcgttgtaaacgatgaaggcacgaagcaaatggtatttgctgaaagttctgtttatggtatctttgaagtagtgcaagaaggctctccaatgattctggagtatcatggcactaataaaccatatactgctatggatgttatcaacaacaagaggaatgcaaaatgattttactctattccggtaaggcaaaatacatgcaaggtgttaaacgtgtagaggttactaagacagtaacaatcgggaaatcttttatcgtttattatttccttgttaatgggaaggagtatgctgtagcaaagaccttggttgataagggtaaaacgtggaaagtggaggcactatgatccctgtagctgggtgggtggtattggcaacacttaatgggaatgttactgtggagactaaaaccatcttcaaagcaccagaagattgctataactccttggtaacttggcctcgtgatgaggatgataaggcagagtatcagtgtttttacattgagggtaagcctgtagaatttgaggttaaagtgaaggaggaaaaatgagaattaaagtaaataaagcttacgatgataacccgaagtttatctctttgaataagagttatggtgtcgaaaacggcgatgagttcgaggttatcgatctttttgaggatcacgggtatattgtcaaagccccaatcacagagcagcctcttatcatcttacgcacagaggctgttgaggtaggttctgatgagcctcaatatgacagcccatacatgacagagtggattgtggctgtagatgttggaggcagggtttctgtattgaaagctcctaatctccatcctagttttacagagaacggtttgacagctttggattacgggttgccagaagatgttgagaatacaccgccaggtatgtaccgttggatttgctctttccatgaagagaaggattgggaatcaggtcttgtggaggattggcattttgaagtggagagttcttttttaatcatgccagcttagaggaggttatatgaaagtttggatagatgatatgcgtaacccagccgactatggacaggaagatgcaatttggtttaaaacgtcacaagaagctatggcttttctcaatgatcctgctaaactgtatcgaagagtaaccgagtggtcttttgacaatgatttaggtatggagtcagaccaagatgggtatgatgttttcctgtacctggaagaacttctggcgtttggtaaaccttgctttggttcacctaccattttcgtgcatacttctaatcccgccgctggggataaattcatgcttgcaaaagaatccatgctgcgttatggtattaaaatgattcgtgtgcactattaaggagagaacatgcatcattcggtttggtcggaacaagaagaggacgaaggttactttatctaccgagaagaagaggattggcccgacacaagtaatgatcttttacttccagatactagttttttcgaggataacttaagagaggagggttaggaaacaatgagtggtattgaaaacaatatttgttaccaacctcttacctggattggtgagggtcattatgcgcaacgtgttatcgtctgtgctgcaaataagtatattttgaataatggtgaagagttaattattccttgtgttcgacatgcaaatcctgtactatatgcacagttaaacgtgttgaaagaagcaggtgtaatcaacaaaactttgtgcacaccagataatcaaggctttatcgatcagtattacaattggtggagtcgtgcggatgcttatatcatcgcggaaaatgctgaccagatcgaccatgagcgtaatggtcacgaccatgaactatttagcgagggattatactaatgtttattacaagtgatttgcattttatgcacaaacgtatacgagagtttgctcctgaatatcgaccgtgggaagatatggaccacatgacagaagcactgatcgaagcatggaataaatgcgctacgagtccaggtgtcaagatgtggcatttgggtgacttctgtttcggaactgaagaagatacccgtaagattgcttcccgcctccaaggggatatcacttattgtgttgggaaccatgattacagcaaacaccgagaagtgcttgcagagtatggagaagtggtgtattatgacgaagtgaaatatcagaagctgttcttcgtcttaagccattaccctatggcctcctggaataagcaaggtcgtggatcaatcatgctacacgggcattcccacggatctttccctgatgaagcttatggtaaaatgttagatgtgggttgggatgcacacggaaaaatcttacattttgatgagataatttctatgatgagtaaacgagatatcgtaactgtagaccatcacaaaataatcaaaggagagtaatgtggctaaatttaattgcgacgatttagttgaaaaaggtttagttcataagaaaacatataaagaaggagagtattctggtctatctgttcttaagtataagaacaaagttttctgggataacctatggaacattgattctcgtcttctagaatgtcgcggaatcgttgttgacgctgatgacaatgttgttatttggcctttcactaagatttttaatcgttttgagaatggcacagatctaccagcggatgaatttgtagatgttgttcgtaaagtaaatggttttatggcctctgtaggtgtatataaaggtcaacttatcgtatcgacaacaggaagccttgaatcagattttgcaagtattgcaaagaatcatattgtttcagagtgtgatgacatagaacacttcatcaaatggacagaaagtgcaaaagcaactttcctttttgaaatctgtgatccttctgatccgcatattgtcgaagaagaaccaggtgcatacttaattggtgccagaattcaaaaccctgatgggacaagttttatgttacctgaaggtgctcttgatggaattgcttctgtcaccaaatttaagcgtcctgaagttcatccgatggttaagttttcagatgttgttgaaatgtctaaagattgtaaacacgaagggtttgtagttcgtgaaacaaactatccgtttaacttactcttaaagattaagtcaccatactatcttactaagaagtttcttatgcgcggtggagataaaaagtgggatctgatttgggataatccaaaaaatgccaaacaaattgttgacgaagaatattacgctttgttagaatggattcgaaataattttacgaagactcaatgggctgctttaaatcagcaagagcgacgtgaaaaaatcgaaaacttctttggaggataaatgaaacaacttatcattttacgtggcgtttcaggtgctggtaaatctacagtagcagaaattatttctgaaggttattggcctatctgtgaggcagatcaatatcattacacagaaagtggtgtttacgattggaagccagaaaatgtaggtaaagcacacgcatggtgtcagagtattgttcgtgatgctatggcatctaatcttaacaagattattgtttctaacacttcaaccacagagaaagaacttaaaccttatctagctcttgctgaagagtttggttatcaagttatctctttagttgttgaaaataggcacggtaatgattcaattcacgaggttccacaatatattcgtgatcagcaagagaagcgtcttcgtggttcacttaaacttcaataaggagaaataatgcagatttatacagatgatcgtaaatatgagacaattttagatgaagatactggacgaattaccgttctccgccacggtgaagagtggcgtgatgaaacgggagataattacatcttgtctctgattcatcttattgaacaaatgcgcgatgattacgctgaactccattatgaaaatgaggcgaataaagtaaaatttgaaaatgagttaggagagaaagaatctgaaatggtatatctaaaagaagagatctcccgtttggaggaagagatcgattctcttcaagaagagatttacgaactcaacaacgaactgaatgcttaaggagggattatgaaaaaagatatcgttgttgttggttggctgacttgcccttgtggtagtaatgacataactgtaaacacagagggtgataaaaattatttattctacggggataaaatcacttgcaatacctgtggacgtgtcggtattatagaaaccgatgattgttgtgcttttgaatcttgggatgaagatgaatgagtactgcattttatgttcgtagcggggacgaggattttctagctacttttgatgacggtgatcttgaagatatgtatgaagatatgcttgactatgccaaagggcaaggtttcacaagcattgattatatgttggcaacatctacaacgttactcgacgcagatttggatgaagtgtgtaacagattgaaaatttcagtttttaaatgggaggactaatgcaacaaacaaatcaaacgctgtatggtaaagattcaaaaggaaaacttaaacagtggacagtgtatacagatggtgcagttgttactgtagctcatggaagggtaggtggaaaaatcacagaaaaatcttatacagcagaagcgaaaaacattggtcgttccaatgaaacaacaccggaagaacaagctgaacttgaagcttttgccaaatggacaaaacagttaaagaatggctattataaaacaaaagaagaagctgaaaaccatgttccttctacaccaatgaaatgccaggattataaagattatcctgaaagggttgtgttccctggttatgcacaatacaaactgaacgggttacgttctatgattaacatgaaaggggagtgtttatcaaaagctggggaagtgtacaacctgccacctcattggaaaggtattccaaactttattagtgaagttgttggtcatcctcttgatggtgaagtgtttgctgggttaggtgtccttagtctacaacaaattaacagtgcctggaagaagcataatgcaaatacacctttgctgaactattacgtatacgatattccagttccagatctaccgtttgaaaaacgtgttgatgttcttactgctcttaaggcagtagtggaggattatggtcttgatgagcttatttgtgtggttcatacagagtatattgaaaaccaagaacagtttgatgctttctatgaaaaagctttacttgagaaagctgaaggtgtagtatatcgtaatactcacggagtttacgaatttgataaacgttcttatgatgttattaaacgtaagcctcgtcatacgcttgaagttaaagtaataaattcaaaaagtgataaaaatggcgatggagtgttgacaggtgagatggaaaatggtaaagtagtcgaattcttaatgcgtaaagatgctcatgagactatcaattatcgtaaagcagagaatgcttctaccttgattaatcagttcgttgaaatagagtatgaagaattttcagatgaaggtattccaacaaaacctgttggtgttcgtattcgagaaatgatcccaggcacttgggaaccagcagaataaggaggaaaaatgttcttagcttatcattgtgcaaaatacctggaggcatttttatgataattttagcaacaattcttatgatgttgatagcaattgctattgtaattctggcatcttttccggcagataaatggtatttcaatattccacgtatgcttgttttcggtttcgcagggatttacatgggtgagactgtagtaaatattagcaactttctagaaagctattttaaataaggagttataatgatcgagaatttttttaaagatattaaattcgatactgaatttttcaaaaatgcagcaaagcacgtaattgattggaatgccaaagcacgaggtggtgaacaacctgaattcactcaagagttatttatgtcccaaatcgggttcgttcgtgaagagcttaaagagtatgaaacagccgcgaaagtaaacgatcatgtagaactcttggatgcagcagcggatatttttgtagtatccagttatctgacttacatgttctttggtgaagaacatacgatgaatttcttggactatgctgtaggcgatgtttttactccaactgtgtatactgattttgatgctttggaacaaacatttacagagccagttcaaatggcactaatggctgctggtattatgcgtatgtctcgtgacactctaatccgttcaaaatatccagaaaagaaagttattaaagaggtattgaaaagtaatgattccaagtatccaactaaacaacagttgctgaaaacttggaaaacttcggatatcgattcagccattgcacaagaatgcgctgcaattgaagaacgaaatgccaatcgtccagaaggggcatacactggtgttcatgctgtttataatgaaaaacagaaagtgtatgtgttctttgatgataaaggtaagatcatgaaaccatcgacctttgtcaaacctaatattgcaggtattattaacaagtaatgttgtaagccgccctttacgggcggtaatgtgagatgtattaaataggaggaaacatgtttgtaaaatcttttaaatccgctctgcctatggttgttggtgacaatgcttctgtagcatccattaacagcattacagatattcctctggaatttgtaacagcagaaggctcccagtcacacactatcggtcttgttcgtttcgaagatgactctaaatttgtaaaagagttttcagatcttgaactgcttaatgtaggtcaatctgtgaagaaggtagataaaaagaaagttaaacgtattttgaatgaacgtctagaagaaatgcgtcaggacatggagagcgaaggtaaagagttttatgtcgataaagaagtagtcaaagcaatgaaggaagatattgaatttgcactattgcctgaaacagaacctgaagatttctttaactatgttgtcttggataaaactacgaatcttctttatgttcttaatggaagtaaaaaggttgcagaagagattacttctttcctccgttccgttctggggactttcccttcggtaggttttagtgtgcatgaagaagctattgtgaaaggttttggtgaattgctggagaatcacgaagcagatcgtattactctcggaaattatatcaaaatggaggatagcgagggcgttgttgtttggagtaaagaatccctatacgagtctaaagctgtcgaactaaaagaagatggtaaagatgttgttgcaatcggtcttgaatatgacggtgttgtagattttgtagtggataaagagtttaaactttctaaactacgttttccgaaatattttgcagacgaagaaggtagtattgaagcttcggtccttctgtgcttcaatgagattcgaggtctgatggaagatttaaccaaagcaacaatccttaaataagttcttgacttgcgggattagttatgatattatgcctacctacttaatgaggtaggctttttaggaggcgttatgaaaaagattctgacagtcgttcttttagcttttagtttcacagtagcagcaggtcaaattactttaatcttgtctgattctgaagttaatggcaatacaaaatattgctactattctaacgctaaccaagatgaagttatagcaatcccgaaaagtcgtcaatgtccgtatactaaaacattctcaacagaggatgacgatgaataattagggggaaatattagcggttatattttaattaaacctgaagatcttcatttatatgtcaacgaagaaacaatagctgcaattgattatgatcagatcgctttccaagcagcatctgcattagagaaaagaacaatcattgcgaagcacatcccttcaggtgctcaaaaggaatttaaaaataagacagagttttggggtaaaagcaagactaaaattggaggttggctaggagatacaaatgcagaaagagcctccaaaaacaaacgtcccttcacaaaggaagagttcgaaatcattgataagcaatacccgccagaagatatcggacacaccttccaagcagctaaaacaaaagtcttaggtattctaaaacatctacgtttagaaaaatattgcggagttattggcgtaggaaaaactttcagacacgccttcaaaatgcctgtagaatataaatctgaacgattagatatcaaaccccttcagctttcagaaaccaaagattatcttgtggagttccacaatggtgatgttgttactgtaatagaagctgatgatgagttagagaagtatggtttccaaggctataaagattataaaaggacgggtaaattttcttatatcgtagtatctatagataaagatagtttacatactcctagtatcctttttaatttctacaaagaggatggagtatttaaaaatccagatataattcttattgacgagtctattggcgatatatggatcgtagataaaggtaaaaagaaagaagttaaaggttggggcagttactggttagcatatcagatgctaatgggagataaaacagatagtatccgaccttatcaagattttgatattaagttcggagatattacttgttttaatcttctttcagcttgtaaaactcaagcagaacttttccaaacagttaaagatacttactactcatggttcccagatggtgttaattttacttcttgggatggagaagaagtccatatgacaacagatgaatggatagaaacaattttccagttggtatacatgaaacgctgggataatgacgatacaacatttgagagtatgcttcagtcatacatctcacaacaggaggcagagtgagcagattaacagaagagcagaaagcagatatccttagtatgagtgcagagggtatgtcttctcgtaatattgcagcaattgttttaggtagtccaacaagaaaaagcacagtgaacgatttccttgcacgatttggagcaagtaaacaagagccagttaaaaagaaacaacccgttattaaaattattgatgttgaaactgcaccagagattgcatatactttccgtcgattcaaagccttcatttctccagatcaagtaattcgtaggggttatttgttatcttactcttctgctgacctgtatactggtgaagtggaaggtaagaacttgtcttcttatcctctttttgatattgatcatacagatgattgggagctttgccaagatctttggaaccttctgaatgaggcagatgtagttgtggcacacaacgggtataagtttgaccgtgcatacattaaccaacgatttgcatatcacgatatggctcctccttctaattatgtagttgttgatacattgaaggcagctaagaaacaatttgcacttccttccaactcccttaaagaaatgggagcatatttcgaaacagaaagtgaaaaactggataacgaggggttccctttatggaaagcatgttgtgaaggtgatcgtgatgcttttgaacgtatgcagatttacaatgatggtgatgttgtttcgctccgtgatttgtatatgaaaatccttccgtggattccacagcatcctaacttatccgcttattacccagatgagaaggtccgttgttctcgttgtggcagtgatcacgttcacgttgttactggtaaacaataccacactgcggtaagttctttcgatatcattcgttgtgaatcttgtggaggtttgtcacgtagtagagcaaacaccagatccaaggataaacgagcagcaactatcattggaatctaataatcagggagcttattgctcccttttttattgacaaaaattttatattagtgtatactctgctaaccacaataaaaggaggaaatatgagcattacattaacaggtaacgaaattttcgaactgtttgaaatgttaggtttgaactataccatcgagaaagacccagatttacttgacgttgaaataacaatccaagaaggtacagcaatttataatgacgatggtgatattatctatcaaggtagggtagcttattatgcagactacccagaagaaggtgcagtaggaattcaggaggaaaattatgtctaatgcttttgataatcaggttgacggtgatcactacacaaaaatggtaatgcaacctattgaactagcttatttggttggtggcacaccttgtttttgcaaattggcaaaatacgcaacacgtattaaaggtgaacgtcagattaacctagataaagctattcattgtattgaactagaaagaacccttatgcaaaatagcacgtatatggttcgtgaaaattatccatctatgacaaatgcagctaaacatgatcaagctatgcttcttattgatttcttcacaaaagatcctttaatacgagtagcactgaaacacatgtataactcttcttatgtagatgcaatcgaagctattaaacaacttaaggaacgtattagtggcgagcaatactaaacaatatggagcaggagaagtttataaaatattcaaccgaaaaggtgataaacttttactaactttgactattccagaaacaatgtgcaaggcggggttagaccgccttttgttttttaaacataatatcccaccaggggaatatacagctaaaaaggtgagcaatattggcaggtgaaccaaaaataatgtggggtgaaggaacaccctggaaaacacaagctgaattttatacttatcttaggggatgttttagaaaagcatggatgagacaccctaataaaattaaggttatcaatgcaaggcgttttaaagttgatcgtgtagataaagacggcaatgtgatgatagacaagaaaactggaaaaccaaaacaagtttggaattgtgagtgcgaaatatgtggacacagaggaggaatgaaagacttccaagtggatcatattcacgcagcaaaacaacttcaatgttttgaggatattcccctttttgttttccgtttgatctatgtgaaagaagaagatttgcgaatggtttgtaaattgtgcaactctattcttgcttatgcagacaagcaaggcatatcttttgaagaagcaaaagctgaaaaagaagcgatagctttgtgtagagctaagaaagataaagcgtggctgaagaagcgtaaaatagaacctgaaagcacagtagcaaaacgccgtaaacaaatagtcaaaatcctattgcaggaaattgaaagttagtataagataactatactaaaagagagaggaggttttatgcagatagtatgtgattgggatttagttgtggctccattagatgttgcttggttagaatggttgaataagattagtggacttggtgttaagatagacacattcacaaaaaatgcagactacgatcttaccaactactttcctggtttcagggaggagttcaatatagatccttacggatttctggataatcctcatctctatgatacaatgggttttgttccaggtatgccagatttcttaaagtatgtttcaaggaaaggtagcaatcttttgatcgcaagtgtgacaaaaggtggtcacttaagttctaaacataaacatgttttaagaacattagatacgattgattttagcaaaggatctggtaacggcttttttgctacgaaagaaaaatacttattgccttgtgatatagcaatagatgacagggctgataacctactatattttccagatgaagtggttaaaatatattttaatacgccttataaagataaccatttagaagaactaaagaaaaaaccaaatgttgtgatcacaggtttagaagatcagtggctaacatgtttggatgtttttaacactttaaacaattttaaaaactaaggagaaaaaatgacaagacagatttcggctagtattattgcgcattccatcactgaagacggaaaagagattgtaacttttgaactagaatatccacgtattattcattcagaattgatgactcaccgactgttcagtcgtaatgctgcttctagccgtgctatacctgttaaaacactgattgaaatggtgcgaaatcaacctgctcgtccttatcgttttggggctaaccaacctggtatgcaggataaaggtgttgatcatgacgctcttattgatgcaggttatactgctcaagagtggtggaatcttgccgcgcttagtgcagcacgtttcgcagaagaattctctttggcagggtaccacaaacaagttgcaaaccgtcttctggaacctttccaacgaattaaaacagttctgacagcaacagaatatgaaaatttctggtggctacgtgatgacaaagaagccgatcctactatcgaagctttggctaaagagatgcacaaagagtatgaaggatctacaccagaagtgctgaaaccagggcaatggcacacaccttatgtagaccatttctatgataatattggtacagaaggtgacgatcattttgtttttggtggatactatgttcaagatgaatctggagaagttgttgtgcttactttggaagaagccaaagctatttcagcttcgtgttgtgcacaggttagctatcgtcgtttaaatgcaactaaagataaagctttagatatctatggtcgtttgctgactgggcgcaaggttcatgcttctccgtttgaacatcaagccactccaattcaagaaacatgcgaacaatatggagagaactgttttgaagatttgtttatcaatgctccttgggattttaactcttgggaaccaggtattacccatgtagatcgtaatggtaagttttggtctggcaatttcaaaggctggattcaacatcgtcaacttttaacaaatcatactaaatggtgatctgtttaaatagttattgactttagtattagttttagtataatatagcacaatactgggaggaggtgagtcctcccattcaaaatgaaggagaaaatatgtcattagtaaatactacaacttgggctaccgtaagcatgtctactgtaatgtctacccctatctggaaactagaatcaaataaacacaatggcaatatcccacacgagaaacaaaatgctgttattagagcgtcgcttgtaatgctgggtatggctgaagattcacactattctgttgagtgtaatgtgaatatccgaagcaataacgataagcgagtagtgttttcaaatacaacgcttttcactttccctgtttcacctaattgtggttttaaacgaatctatcaagaaaatgatatttttcattttggtgatgaagaatttacaggttggggtgcgtcacatattaagatggatgatttcggtaactcttatgatcctgcgaaagcagatgatactaatcaatgagagggacaatgactgataataaatatgaaggctgtgaaatcgaagtctattttgatgaaactgttggttgtaatgtaactgtaattctagacacttcaaaacgtttatctcctaaaagacaagaagcagaccgtcgagcaaaagcaaaagggtttgatgacataactcattttgaaggttttctatatgctaatggactttttaatgaaatgaagttcggttatgatgtagatgttttgaatatgcattaataatagggaggagaagttgattcgttttgtaactaaatctaacggtaagacagaggagtttaatccagaaaaacttcgcaagtggtctgaatttgcagcaggtaaactaaactggtctgaaattgaattagaagctgtgaagcggtgttatgacggtgtgtctacacgtgatctgcataaagcaatgattgcagcgtgtattgacaagaaaacacaagcttacagtgatatggcaggaagacttcttctgggtatgatttacaaacaggctcatgggggataccacagtatcccttcactgaataccttttatcatgatatggtagaacgtggacattgggctaaaatggattatactgatgaagagatcaaagaaatcggtaagattgtaaagcataataaaaatctaacttatggttattctgttttacgtcagatggtagataaatatcttattcgagatgcagtaaacgatgtggtgcatgagtctccacagtttcttttcattggtatcgcaatgaaggttatggagatccaaccaaaagaacgccgtatagaagatattaaaaaactttatacttatgttagcgatcttaagattaatctgccatcgccgtatctgacaactctacgaactccaatcaaaggaagtgcgtcttgttgtttgttccgtgcagacgatacggcagagtctttagccattgctaattacttagctcatgagtatacgttgaataacgcaggtattggtgttaatcttcaaactcgtgccacaggtcaaggtgttaagaataatcgtattattcatggtggtcttcttccgtatctgaaatggatggaagatagtgttggagcttcgaagcaagcaagccgtggaggtagtgctacagttacctttaccatacttgaaccagacttcgacgatctgattcgactgaagaatccaacaacacctatagataaacgagtagacaaactggattattctattgttgttaaccgttcgttcttgcgtcgagcagcaaaaaacgaagattggatgttagttagttgtgatgatgctcctgaactttatgaagctttgtataaaagtgaagaagagtttgatgcagtttatgacaaagtaagccgtcgtcgcattaagaaaaaatttgttaaggctcgtgatcttctgacagagattattgttcaacgtgcagaaacaggacgtatttacatgttctttgctgacaacgttaacaaccatacacctttcctggatacggtgtatcaatcaaacctgtgtcaagagatcttcttaccaactaaagctttccacagtatgccggatctctttactggtgagaatacagaaaatcaagaattggctttatgcttcttatcttctattgttgctggtcgtgtcttacctgaagaatatgaggatgttgcatactacactgtattgacaattgacaacgttattgaaacaatggattatccatttaaacataacgaaataactgctaaagcacgtagatctattggtgtaggcattacaaacttagcgcattatttggcaagtaattttgtaaattattcagacgcttatgctaaaaaattaatgcatgaacttgcagaacgtcattacttttggttggcaaaagcctctctgcgtctggctaaggaaaaaggtaatgcagaatggatcgataagaccaaatggcctcaaggttggctaccaattgacacttatgctaaaacagtagatagtatcggtaattttgatcttaaaatggactgggaaggcttacgtgctgaaatcattgaacaaggtggtatccgtaacagtgtcttggctgctattgcacctaatgagtccagcagtttggtaagcaatactactaactctctgtatcctgttcgtgatactattatgtataaacaaagtcagaaaggtagtgtattatttattgttccagactatgaaaaactgaaaatgttctaccagtctgcctgggacactccggctaaagatcttgcagaaatgtatggtatctttaccaagtttacggaccaaggaatttcttgtgattcttggctagattattctaaattggtaggtggacgtttatcagtgaaagctgagatgcagtttattctgaattttgctagactgggtggcaagtcattgtattaccttaacagtagaacaaaatcagcagactctttagcacaagaagattctgatggtggttgtgaagcttgcaaaatgtgatgaaactaggggcttcagcccctttttattgaggaggattatgagcacacttatagttttacctggagtaaccccaatctaccgacctgggtttaagcctctgttgattcgccctgaagattttacccctacttggtgtcaccattatcttggcggtcttgaaatagctaacatgcagatttgggaatctttttatccagaagccacggcttacgcaataacaagggcaagaagtgaagaaacagtgactgttatacttggtagaagcgtatgggaagcactggaagctttctaccggagcaatgattttgaggaagaagatattttaagcttaagaggtttttgtgaaatagtggaggatttaaatggcagcaattaatttacataacgagacttacaaaacaggtaaatatccagttttcatgggggaacagttggggatttttgattctatcaacgtgacttaccctgaaatgcacaaactctacaaacttcaaaaagcacaagattgggatgaaaacgaagttaatctggatcagacacgtaaagatttcgtagcttgttctaaaaacaactatgatgttatgattaaaaacttatcgttccaatgggaaaatgattctcttgcaaaatcaatcatcactttgttctctccattcttgagtaacaatgaagcctgtgctatgatgatgaagcagagtgaaatggaagttctccatgcgttaacgtattctgaaataatccgtcaatgtactccagatccaattgaagtcatcaatcagattatgattaatgataaaattttcaatcgtatgggtatcattgctgatatcatgggtcagttggaagaagctggtcataagtatgctctaggattgattgaagctaacgatgaacttcgactgctgattcttaaagggatggtagccctgattggattagaaggtattcagtttatatcttccttctctgctacttttgctatggcagaacaacaactgtttgttggggcttgtaaactgatccagaaaattatgctagatgaaatcttgcatgttaagatggattatgctgtaattgatgcattgatgcatgacccaatttggcgtaaaatttacgaagacaatattgaagagttgactttgatcctcgacactgttgttgaacaagaggaacagtggtgtgactacctgtttagtgaaggtcgttttattgtaggtcttaacgctgaattgtctaaacagtgggtttatttcaatgctgctcctatttatcgtcgattgaaaatgaaagccaagtttaaagcacctaaacatagtcctttagcttggatggaaaaatggttgaatcctgatttgattcagtcagcagcccaagagattcagttaactaattacaaattgaattcagctaacaaagatctctctgatgatgaggagcttgacttctaatgattttgagatgtgttggtataatcgacacattcaaagacgtttttgacaaaggggcttcttatgaagcctccgaaatgaagaatggtttttgtgatgtattcggtaaacgcctgaagaaaaacggcgatccttggctaggagttatgtcttgtggtcatattattgtaaatggtgttgccaagtttgaaataattaaggaggaataatggctgaaaaacttattttttgtcctgtgtgcggtgggactggacagatggaagtggaaattatggtaacatttccttgcctgaattgtcatggtgagggacgagtagtaatctttgaaaaagaagggttggaggtagaagatggagagtgaaaaagaatatgtccgcattccacgagaattaacagatcacattgcagaagcaatagctttcgaagctagatgttgtggaggcattgcttacgatatttatgaagcaattattaaagctgctcaagaagaggaggagatgaaaaatgggtattgatatcgaatggcgtatgttacagggtgcaccttttaataaatgggaagaggctgtaggagaagatcctagaattgaagaagaatttgatggagattttaactggttcttacaagaaggtctggacttaagccgcatctctcctttctacgatgcggatgagtcttattgcatatatggccttgatttagaaggcggagagggaagcatagctgtagatcttgagcaattgaatgagaatgcaaaagaagcctatgcaaagctacttgaaaaatataacattaaaacggaaactattatttctcaacatgtctggtaaggagaactgatgcgctatctggtaacaggtggtcttggttttattgggagtgccgtggtgcgctctctttgtcaagatcctaataacgaagtttttgtcgttgataagatgggctatgcagcagatattgagaatgtaggcgatatcactcacgttaaactaagcatgacagatttggcaaacgaaacccaggtcaaaagtttagtagtagagtgcaatcctgacttcatcattcacttagcggcagaaagtcatgtggataattctatcaagaatccacgagttttcatcgactccaatattatcggtacatacaatttattggaagctgcaaggctcctcccaaaaaataaactgaagatgttcgtacacgtctccacagacgaagtgtacggcgatatggcagactctggcgttccctttaccgaagagacagcttatgccccttcaagcccgtacagtgcgtctaaggcagcatctgaccatcttgtgaaagcttggcaccgcacctacgatttgcctgttgtgataaccaactgctccaataactttgggccttatcaacatccagaaaagctgatcccaaaaatcatcacaagtatcttgacgaataaagagattccggtgtatggtaaaggcgaccaggtgcgagactggctttttgttgaggatcatgcgcgagctattcttcaagtggcaacaggtggagaaccaggtcaaacgtataatgttggaacacgtaatcccaaaaccaatatggagatcatctatatggtttgtgactatatggatactgtacatccagaagcagtaacctacccttcttccaacttgataacttttgttgaagatcgtaaaggacatgacttgcgttatgaaatcaatccttctaagatagagtctgaactgggctggagtgcaaactctacatttgaacaagatctctactcaacgattgaatggtacttaaacaaccctcaatggtggagtgagaaaatttaaggagggttatgaaaggtattattttggcgggaggaagtggaacccgccttcatccggtaacacgcagtgtgagcaagcaattgctccctgtgtatgacaaacccatgatctactaccctttatcggtattgatgttggctgacattaaggatattttaatcatcaccacaccaggtgatcaggaagactttgagtatcttctcggagatgggtctatgtttgggataaaactcacttacgcaatccaatccgagccacgaggtcttgcggaagctttcatcataggtaaagattttatcggagaagactctgttgctcttattttaggtgacaacctattcttcggacaaagctttggtaaactactgattaacgccagtgagatggtagatgtttggggcggagcttgtattttcgggtatttggtgaaagatcctgaacgattcggtgtggttgaatttgggagtaatcgtcaagctatttctattgaagagaaacctgaaaatcctaaatcagattatgctgtcacaggtttgtatttctatgataactctgttattcaacgtgcattagaagtaaccccttccgcgcgtggagaactggagatcactgatatcaatcgcagctacctagaagacaggaagttacaagtggaagttctcggacgaggatttacttggcttgacacaggaacacacgccagtctgatggaagctggtcaattcgtgcagactattgagaactgtcaaggattgaagattgcgtgcctggaagaaattgctttcaaaaaagggtggatttctttagagactttggtgtatcatggaaactcactgaagaaaacagagtacgggcaatacctactcaaattagcggagagaaaatgaaagtagaaaaaacaaaaattgaaggtgcggtaattatcaaaccagacgtttataaagatgatcgtggtttctttctagaaacatataatgaacaaaaatacgaagatattattggtagacaaactttcgttcaagataacatgtctaaatcaaacttcggtgttttgcgaggaatgcactttcaaacaaataacccgcaaggtaagctagttcgttgtgtaagtggggaagtattggatttcgctgtagatttacgtaaagatagtccaacttacggtgcttgggacggtgttgttcttcgtgcacgacaccatacgcagttttggcttccgcctggtttggctcatggttttatagttttgagtatggatgctatctttgaatacaaatgcacaactccatacgacccttctagtgaacaaagtttacgttgggatgatcctgatatagggatcgattggggcttcgatatgatttttgacgaaggttttaaacctattctatcaacaaaagatgcaatggctaaatcttttcaacagttacgtagcgagggtgtatgctaacatatctggtattgggcggtaatggacaactgggacaagctttacgtaaagtggctccttcaaatgtatctttaatttatcctaaagaacgtgtagatattacaaattttgaagctgtgcgaaacgctattattgaagattatgtagatggtgttattaattgcgccgcttatacggctgtagataaagcagaacatgattttgctactgctttggctgttaatgcgtatggacctgcaaatattgcggcagtgtgcagcaaacttaaaattgattttgtgcatatttctacagactatatttttaattattctcatcctatttttgaagatactacacctaacccaataaacaattatgggtatacgaaagaacttggtgaaaaaatggtgcgaggagttaacccaaatgcatatattattcgaacagcctcggtatatagcgaatttggaaataactttctaaaaaccatgttgtctcgctataatgatggacaacgtgattttattattgttgctgatcagttctcttgtccaacatatgctccagatttagcagaagctatttttgaagtattaaaaacaggttggtcaaggtctgttaagacttaccattttgcaggaagccatcttatttcttgggcggagtttgcggaaaagatcttgacacagatgccagaacctgttaagatatcggcggtatcagcttcaacctataatgctccagcagcacgtccggtgaagtcacaactagtaaccagtcactatcttacaaaatcagatttaaataaaggtattgaacagtctttagcagtattacttaataaggagaggtaatggaatttgtaatttttggtaaacctaactgtcaattttgtgaaatgtcaaaacaactattaaccatgcgcaataaacagtttgaatatttaagtcttgatcaagactatgatttagaatcaatgactaaaacagttgttgatcttaccggagttcctccacgcacatttccacaaattctgtccgaagtgaacggggaggtaacatacgtaggcggttttaccgaactgcgtgatctcatggctcaaattgcggagtaacaattatgaaaagtaaacagaaacgtaagatcttttctcgtgctttggcaaatgagctgaagtccaacataaagaaaacgaaaccaaaacgtgaaacttctccagaacatctgttgtttaacgagttggatcacggagtaggagacgacgaataggaagtaaatggctaaagtagtagatgagtttgatatgctacgagtcgatgaaggactgaagcttactgtttatcaagaccatcttggttattggacagtcggcataggacaccttctgactaaaataaaagataaagcaaaggctattcaaatcttagataatcttcttggtcgtaagactaatggagttatcacagaaaaagaagcccgtcaaatctttgctgacgatgttgcaaaagtgaagagagacataaaaaacagtgctatcttatcaccaatctatgataaggttagtgatactcgtaaattgggaataatcaacatggttttccagttggggttaaaaggtgctgaagggtttcaaaatagcttgactttaatcagcaatgcatattatactcaagcaggtaaaaacatgcgtaaaagccgatggtatagccagacgccaaaccgtgctgaacgagtaattaaagttctgacttctggaactttagatgcttacaactaataaaggggcttcggccccttctaaaaggagtgctactatgggaaataaaaccgcagctaaaacacaacggcagaaactggctaattcagcaaagattgaaagccgttatgcagcaaaagcggtaggtaatgacgattaccgtttagatgaatttatgcctgaaggggaccaaaataagattgttcaaagcatgatttggaatgatctgactgttgtaaatgctccggcagggactggtaaaacctctgttgctttatggaaagctttgcagatgcttcgttcaggagacttccgtaaactactttttatcaagaaccctactgaagtcggagatgaccagattggtttcttgtctggagataaaacagataaactagcagcacactatgaatctacaaaacgtattttccagcaattcatgacagccaacaaattggaaaacgatattggatcaggtaagatcgaattgaatattcctaactatttgttgggggccaccctggataatacaatcgtcattattgacgaagcacaacttatgtcaacagaaacacttaaacttcttattgaacgtgcaggtattaatacgcatgttattgtattaggcgatgttcgccagacatatgcaattaaaaaacgtagcaatggtctgcatcaaattttggaacttttatatccagaaaatgaagaaggtgttcgtgagccaattgtagattttattggatacgttgaactgacaacggacgataataaacgtggacgtttgtctaaactgattactgaagtgtttatttaatgacatggcagggaagccatttataaggaggattgatgggtgtagcaacaatagctatcggaagaagcggtatagatgagtgctatcaaacgggactcgtaaaacctaacgaatttcgtcaaatttgtgtaaaccatattgaaaaagagattgctcttactttagagaatatgaccctccgagataaactggaaatgtttaaatgttggtgtctaggagaagattggcttttaaaccattttcaagaactttctgatagtaaagaaagcttgtttgattgtataataggaatagctggctccgatcctagccttctgcacataacagaagaagaggcgaagttactgttcttaagtaatttgtttaataagaaagcactgcttgttcgtgattgcagtgatcttcttaataacccgctcgcaacgtctgatcaaatttttgttgacaaagagacactacgtgcgatagaatacatctgcaaatatgtgaatggcgggaaagcggttcttaaaacattaacagaggaggaagaatagtgacgacaattattgcaaccaaaaataagattttgagtgacggtaaggtaacttttggtggacgtgtagattctattaatttcaaaaaggtccgaaaaattggagattaccttgtaggtggtgctggtcgtctgacctccgtgctttctttctttcaatggtttgaacaaaatctacagtgcgaagcagcacgtgatgctgtccctggtttgatgattcactctgatccagataaagaagatgaagagtttattgcacttgttgttcatccagacggacgtattttcttgcacgaaggtaacaacccaacacgtgcatatccgattgatgaagaatattatgcagtaggaagtgggtcagactttgctttagctgctcttgacgcaggggcaacaccagaacaagctatggaagtagctaaaaagcgagatgctttttctggtggtgaaacatttattgaagaacaagacgagattattgaattaacagacgatgatttagaaaacttctccaaagaagaattgatcaatcttttgaaattcggtaatccctacggtgaagaagaacctcgtaacgtggcaggtaacgaggcggatactgaagaaaaatctgttgcagaaaacaacttgacttctaaatctgaaaatattaatctcgtaaatgtggcggatttcgaaggaaccgctatttaacaaactgccccttcgggggcatttcttaaacataggagattaataatgttacaagttcctgctaagtttgttgaaggcgctatccactcttaccgtcaaacatgtaagagggcaagaaactgtttagatggtctggtggcaaaaacctatactgttgaaaagaagaaacgttggtttggcaaagatgtttacgaagttaatatttttggtgaacatacactaaaccacgatactactgaaaacatgttagaaatatttctaagagatgacaaaatatctcctatggattaccaacttttaaaattttatgattcttctttggctattgataagtttgttgaagatattctgtctagaaaaacagataaaaacacttatcctctagaggctgatcaaataggattattaaacgagatttcatcctttaaagagttggataatattacaaaagaggagattttagatggcattagattatgatcgcccaacgagcgattattatccgacaccgcaaaaagcaatcaatagtttgtttagtgttatagatttcaaaaatctaaaggaagaaggttgggtttttgcagaaccttgtagaggtgaagctgaagcgatttatcgccattttcctgaaggttcagaatattgcgaacttgaagaaggtaaagattattttagtcataaatgggaaaatcgtcccgatattataatcacaaatcctccatttaagttagcaatggagttcttggaaaagagcttaaaagaggcggatgtttgtatttatcttcttcggttgggcttcttggagtcaaagaaacgacgagagttccatcaaaagaatccaccaacaaaccttatagtcctttcagaacggccttcgtttgttgcaggtggaacagataagacagcatacgcttggtatgtatacgacccaaagaatcgtttgggcctctctcaaccattttattttgtttaaggagtgtgaactatgggtaaactaattatgacagtggctattgattttgatgaagtagatgaaagcctcaacctttcactttctactgacgtagatggcgaaatggataatattgaagcagctactgctcttttcttcaaaaaagccattgaagtttatgccgaacatgctgcaaatcaggcagtagattacggcggtatcgtgcgagctttggttcactctaccgagggcaatgactgggaagactaatggcttacaattattactgcatttatgcacatgaaaagaaaaaaggtgacactgggactaacgtttatgcggagtccgaatctcatgcaaaagagattggtgctatccttttatcacgctggactggaaataattacagtgcagaagatatgtttgcagaagtggattctagaatccaagaagacaacataacaactacgtgatatacttatactcccttttcggagggagtattttaaggagggattatgcagagcggagaagaacttttagagaaacaattctcaacgcgtcaacaagctttaagttatatggtttttctaagcaatcttgatgtgaatgagatacttttacggcgcgaaggtaatttctggagcgttaaatatcatgcaacaaggtctgtaatctacctggaagaggcggaattctctttggtagaatgatccgaggagtagttatgcgttcaactttcactggcaaaataaatgctggtttgatttttacagcagtaagcattcttagcaaaaagtttaaagttgttaacttacctgtgtgtgtcgatttcgatcatgtttttgatccttcgaaccccactcgttgcatagtaaggagtaatgttgaaaacccaacagaaaatcttttttatgtaacaaaaccatctcaaagcatacttaaccaggttgaggcgaaattgatacctaatggtaaatattactcctttttaccttgtaagtcggttgaagtagagaatagcaatactttcgctaataacttgtctgtattcgttttctttgtttcacaggaagacgaaacagatgaaatggtagcaacagtgagatcactttatgactctttaggggttgtttatagtcaacaagtgtgcgaaaataataaagcctgtgtagatttcattatcaacggtatttgtgtttgtagagtagaatctttttggctgcacgatacatatgttactgtagcagaggttatatctgaaccaagatttagtcatgcggtttcgttaataatttgaggaggaaggaatggaaattactacagaacaagcccttatcgacgagggtcgtaaaatcttagaaaagtatttaggagagattcataaaacccctggctatttgggttggagaattgttgaagctgtagatttccttcctcgtaacagtaaaagcgtagaaatgcgatctcttttcttattagtaaaggtggatggggatacgccttttaaagatgccgcagagcatattgaaaaagttgaagagaaagcttatgcagaatttgccgatcttgttttagaacaagagattgaaggaaaggtgaatatttttattttcttagattttgaggaggataagtgatcatacatagagaaattactgttaaaagcgatcaagatctttttataattggagatctacatggttgttataatctgtatgaaaaaggaaaggcaaagttaggtatccgagatacagatgtggttgtatctttaggagatcttactgatcgtggaaagcaaaactttcgttgtgttttagaatttactcgtaaacacaatcgctacgctattcgtggcaatcatgaagacatgatgattaaaggtatgctggaaggagatcgtagctactacgagtgttggtatcaaaatggaggatatacggtatttgaagaatgtggtgaagaaggagctacagccttagcaatgatgacagaagatttacctgttgttctcattgttaactacagaggcatgaaacttgcgtttattcatggtgggtatccatcttctttggaatatctccctgtaacagatcttccagaatacatttcgaagatgacaaaaacacaggaaaaccgttttgctgaagcacttatgtgggacagagatatggtagactgtgctaaagagggaatgaaacttcctgttgtgatgggtgtggattatgtgtttcatggacactcttatgtaaaaacacctattatcaatgcgaatcgtgtttatatggatactggatctgtgttcaataataacttaacatttgcttatttcgatatggatggtggtcttcaattttattcaactcttgaggaggattaatgtataaaattacaattattcaaagtgtaggtaatatccgtaaagaagttatcttagaaacagaagatgttaatcttgttcgtgagattgttcttaaggaagataacctgcaaactgtagaatcagatgcaccgaatcttatggtggatcaagaatattctgaacttcgtcgtatttttgaagagattcagaagaaagaaacacaacctaccataaaaccttttggtccttatgatccgagcaatcctagcagtccttggattatgagaaatacggaaccctatacaccttttgaaattacttgttgaggtgattatggcagaactgttctatacaggagttggaagtagggacatatctgaagaagagtgggctttgatggtaggcatagggaaatggctttcaaaccttgggtttaaattacggtctggtaaggcagagggtagtgattctgcttttgaaacaggtgtccaagagtctgaaaatcctactaacaaggaaatttatgttccttgggaaaatttcaaaggtaatcagattccaggtgatgttattgttcttgcaaaaccagatacaatgaactatgctatctctgtgcaatggatcaaagatgttcacccagcttatgaaaagcttaagcagggtgctagaaaattgcaccaacgtaatgtgcatcaagtgttaggcagagatttggagaatcctgctccatcatgtttcttgttggcttgtgctaatactgataaacatggagatgcaaaaggtggaacagctacggcttggaagatagctaaagccaataatgttccttgtttaaacattcgtgggaaaacaaaacaagagatttacgattttctacgacctttggtaatgagggcaataacaagtgattcctagacgtaaacgtggtgaacaaagaacaaacttggcagaaatcttagattacaacatggttaatgatgtgacaaaagaaattctccctgttgtagaagatattcttcgtagagggttacgattctgctcggatcgggtgcatttcatgtggacacataaaagtgactcctctttgaatttttatcctttagactatactctgaacaatacagagatgaaagatatcaacaaatatttaatgggatataaagatataaccgaaactgttcttggagaattggcagaaatcttggctaacgatatccttagaaatattaataaggctcttgacagaagcccagcaggagtagtagtatcttttgctgtgccagatatggttaaatatgagtctacaatacctaataaagcttatgcctttcttcggagagatgttcgtggaaatgtgttggagatggagtcaaactacaagattgaggaggttttatggcagagttcggatcagtaacaagttttgaaagtaacttcgttaccgcttgggaagcttgggatgaaatgggtgttggggaactttatttttacaatgtaacactacgtccagagtatgcttacctggtaccagaaggaacagttcaaattggcatgtatttgtcttctcaagacggtgttgttcaatttgagttttattctaacacaacggacaacgaatttacataccgcgcatttcatgtcaacatgtctttaggagaggagaaattttatgacgaccaagtttaaaacagtagaagaagccgtaaaagcagcttatgaggctttagcggaagcagaacagttagcagacgagactggtgaaggtttcagtttctctcctgaatatgggatgggtggttattatgatccagaagatgaaaatgagaatactggaacaaattggcatccatcttctttaagttgttaataggaggtaaagtgggaatcgaagatatcaaaggttataaaccgcataccgatgacaaaattggtaaagtgaatgctattaaagatgctgaagttcgattaggattaatctttgatgctctaatgtctgaattgtgggctgaactggatgattgtgaagatttggaagagtctaccaatatatccacccgccttgaccaaatttcaatagcaaagactaagttgaaagaagcttctatgtgggcctgtcgttccgttttccaaccagaggagaaatactaatgagttatcgtaacattgaagaattaactgaaaaagcccaagctattatggctttagtggacgaacttacaaaagaagccgaagatgctggttttggtcttagcttagaagatgacgcaatctgttttgaagattggcaatcctcttcttgttatggtgaaggtgatgatggtttcgtcgttaattctcaaggcgtttggcacagtagtagctgttaaggagagattgtggagtctgaaggcgaactgctgataaaaaagaaagttaaagagatggaacaagcacaaagaaagtttcgtaaggaaattgaagaactctgtgaacagtttcaggaacaagcttggataggtgactacggtaacggagaaacttattaccctacaggaacgccagccgcagatcactatattgatttcatttgtgaagattatggtgtagagttggatgaaaatggtttaaccacaagaggtgtttggatctcttcttcctcaatgtgttagtaaataataacccgcttcggcgggttttttattgacaggaatctggaagcttgataaacttaacctatcaaccaatagaggagaaatattatggtgttaattggtagccgtgcattacaaattcatgctatggacatgggaaaagacttgtatcgcccttgtgtagatttagattacctgtgtacagaacaagagtgggaagaattatcaaaaatgttctctcaagacgagtttgttgaacttgtagaacgtaaagggaacaaaggccacgtcaaaatgatagctggtgcacatttggaatatgatattgcttctcaaggagatagcacagaagctttgattagttattgtgaagattctgatgatgttcgtcgaacttacgctggacatgttgttgctcctttagatgtgctctaccttcttaaaatgagtcatcgttttaaaaaggatagtcctcacttctttaaaactatgaaagatattcatttcatgcggtcactgggcgcaaagatcccagaaaatcttaagagtatttatgagttacgtgagaaagaaacatacacttactctcatccgaacttaaacgtaaaaagtaaagacttctttaaaggtgacgaggttccgtatgtttatgatcatgattctatccatgaagcaatcgcgttaatggatgctccagcatataaatcttatatgaaagatggaagtgaagttatgaccagcaaagaaaagttttttgctcaaccaaaacatgtgcgcctgttaggtgtttatgaagaagcgtgtgttttggctttggaacgttcacaaattcctttcggtgaagagatcggaggtccaacccctaagcaaagttttataatggctttatctaaagtctgcacatccatcactagtggttggtttagggaatatgcttgggaaaacttttatgttgttactaagctatatgaaaacatgggcgaaaacgactatgttgaacgttttaaagctaatcaacatgtagttaagccttttgaaggaggttattaatatgtgtggatatctttcagaaagttatttcaaagtgacccatgaggatttagaaaaagcacgattgaaactgacagctattattgaatactctaatactgttgatcacagattaaagactactaaagtatctgttccttgttggttaggtttacggcataaaacaatgtctcagcatgaccatgttgttcgagagatgcatggttggtgttggtttcacgatgtggctctgcattttggttatattacagaagaagagcatagattctgtgaactaacatatcgtattccaacccttcacgatttagatttgtggaagaaagcaaacgagatttttcttacagaaagtgattggcgtaaagttatggcttgtcttgaatggaggggtaatgagtaaaacagttttgtctatgtttgacggatctgggattatggtgttacgttatgccgaagcaggtcacaagtgtttttgtgtaaacgctgatgaaggggatcacggtaaatacgaggttaggatggagcacccaaacataacctatgtgaatgcctggatagattctacattcaaccctgaagatcatgggatacctaaacccgatattatttttggatttccaccttgcactatgttagcaggtagtggtagcaaacacgaacgtagcacagaggaactagatgaagctttagctaatattcgtgtagtggaagctttgggtaacaaatataatgttccttggatgattgagaacccagtaggtaaactttcaaccttatggcgtaaacccgatttttactttgatccttatgagtatggtgggtatttgtttgaagaggaaggaagctttcatcctaaaatgcctacccgtgatgggtatacaaagaaaacttgtatttggcacggcaatggttttgttcaaccagtgaagaaaccagtagatcatataggctgcttctggggttggaaaagcttaggtggagctagtgcaaaaacaaaacaacttagatccttaaccgcacgtggttttgctcgtgcagtgtttttgtctaacaatcttctagataaggagaaataatgtcaaattcaaaacaagtagttgcttatcaagtagatcgtaatgatggtgaaggaagtgttgttattttcagtgcacacggtcttgaggctcgtcgaaaaggtgcatatcgtttagaatgtgagtttgaagactgcacaagccaccgtgtcaaacaattcgatcaatatgctgataaaggcttcgtccctatcaaagcgttactggaagatggttggtgggtatattctgcttttgattactcaaaactggaggagtcagcttcgtgcgtctccgagatcgaatatgatgattactatgacactctctaccttccagaatggcttgtcttttctcctgatgagagaagtgtctggaaaacgtgggatgaaatggaacgccatgcttattttataaacgaagcattagatcgtaaaacttggtttgaaaatacagttaaagcagcgtatcctcaatttacttttacagagttttggggtggcccacaccatattactcatgtggcatacttcgagttcccaggctcacgctataaaggtactgtctattgggattgggacgaggacaaagaaccttctgctcaagacttccgttgctatgtttgtcaaggtgatcaagaagcacttgacaaataccttcaaagtctggtatagtgccgatatctgaacaaagcctcctgtttcagggggcttttcttttataggagggaatatgtttcaagtaaatgtcaatgtgcataatagttactctgattgtgaaacgtgtggctatagttcatctaatgttgttcgtattagtggcgatttaggagaagcttcttttggagaagaagcaagttgctttagtggtatggatgcagaatggaatgatattgcacattggttggtgagcaaactaaaagaaaaagagaaaccatttcctactctgcttgatcgttctcttgtggaagatgcaggtaaagcatatgcagactatgcgtatgcagacgggaaatatttggaagatgataaggctaaccatttgctttatgtttactatgaagctgaatctgcctacgataattattacagcatgtctaacatcctacgtctgtttgcagagtttgatatttatttcgaagtagaattttcagaagatgatacttacgagtacgattcttatgatccatttgaggaggaagaatgccaagacgattaatagtgattagcggcgctggcttaagcgccgatagcggtattcgtactttccgcactgacacagaaagtggtaaagctttgtgggatgaatacgacctttctgaagtgtgtgacattttcaatttccgtgctggctaccagcaagaacagtgtgttcctggcgctgttggtggattagatgcagagggatacaacctctaccgtaaaactcatgacttctacaataaacgtcgcgtagagatggatagtgtgtctcctaacgctgcgcattaccagatcgcggaatggtttcaagagtacggctgtgatcgtgttatcaacatcacgacaaacgtagatgatcttctggaacaagcgggtgttgttcatgaagatgttctacatgttcacggcttcctgcctgaagtgatcgtagacaatagaatcatcaaagatgttggctatgctgctatcgatgtggatgattacaagtatgtgaagcccaatgtagtgttctttggggagactgccccattgtataacgatatgtaccacatcttagacggcttgacggctcaagatatggtgattgtagtgggttgctctaatcaagttatcaattttaactgggagctttttcctgctcttaatatcgggacaaaaatggttgtagttaaccctggagttaattacgaagaaacacttctctatgaagatcgtggagtttttgtttaccgagaaaaagctgcaacagccttcagcaatgaaggtttcattaaaatggtcgaagaccacttaaacaaataaaccactcaaggggcttcggcccctttcttattaggagaataaaatgactgaagaaatccgtgattactaccttgcacattacgtttactactttgctaatgctacacaaaaaggggaaggtaatctttacttggcaacaggacatggaacaggcgttaggaaatcaaatattgataatgccttggctcaagtggctgcggggctagaagataaacttcaaacacctgtagttgtagtgatgcaaaatataagttacctagcaagttgtactgaaaccgaatttaacgcgtctgagacgcaatctaacgaagaataaaacaaaggcacccaagtgggtgccttttctattttagagcgtctctgttcgatttaggtgggttctagcttacttgtgcagtgctctctcgtataactcgatgatcaattcatcctctactgtaatacgcgccatttgatccgtcttaagtattgttggttggtagtggtgcatttcactattgatactacaatcccattcgtcataacctaaatactctcctacatggaccttaaccataaagggaaacgggttatctaccaaaaatgccatacaaccttcttcgattttatttaacacacgacctcctattgctacagatatgaaaaaacccgccgaagcgggttctattgctacgataggatcactatcctatagtattgttcctgtgtcaagccttagcgtagaaaaacttctcatattcagcttttgttacgattgtggggcaatcttttgtcccattttcaataacaggagcttctaccattctggtgttgaataagcaataagttccacgcatgtaaatacccagttttgtttctacgatgtgctctttacttacgcttgaaaacttaatctcaccatcggagtctcttatagcaataagcgcatgttcgggccaatcttttccttttgctcctaaagcattatacaattcttgaacgagttccatatgacctccttattaaataacaaatttttcttcttctggcttatcgtaatctggagaataaacatgtgcgggattaaaacctttaaaatttccgcgacctttacgtggacagttgaatgtgcgagcttgatgatccccaaaagatttcccacaatgcatacatttaaaatcagtgcgtttagtcatgccatatctccttttaaaaggctgcacctattgaagatgcagctttgatagattatttagatgagtctaaaatgtcttttacttttgccgtgataacgtcttgatatttgttaatcagatagcctacaactacacctacgataaatgaaaccattatttctccttatttaagtggtgagagaacaatctctcggttttttaagaaccaaagcatgtcatcttgatcatagtctgatccctctgtagtagaatttttgatagaatcaaaaaaccaacgaggtataccaaaacaacttccagatgactgcatgtctttctgcgacattttaaatgtgattggaaaatcacaattatcaatctctcggatatctgctgaagataagattgtaacttcaaccagttcgtcgttagatgtagtatcgttgtcttcctgaataatgtcaaatgctttttgcacatcttttgcatctaatgttaaacctgtggctttctgaaaagccgaaacaaaaattagagcttcttcagagcaaggggcttccagcaaatgagccataataggtgacaagcaaatgttaagatctttgtcataccagcgaacaatccacacaggcgttgctccgttttgtttgtcacgagccaaaattgtaacgtgctgaccttcttctggtatatgatacaaaccagaaaaaccaggacctgtagtagattttttgacacgtgctacgtgtccaacttctaaaggatctttaccatcccaaatcttctcttccatataacctcctaaataacatctttgaaatagtgaatcacttcaatattatggtacaaagtgcttcgctcttttgtgtactctgttttagtaacaaacacttttgcaataccgtacttatgtgctggtaggtttggattggtttgcataatttctttcactacacttatagtttcgtgctcggattcccaataagtgaagtaaccctcactatcaacgattaaagactcgttttcaagatctacaaaacagaaaaatattgacggatggctcattctttttcttcaaccttcctcaaagctttttgatcgaatatagcgtgttgcaactcgccgttttcatcaaaccaagttactttacaagattcaccataactttctattactgtcatttgtttgtttgcacctttaagacaaaccacatcacctacttgcatcatgattgagttcctttttgtttttcaatagctgcacgaatttctgctaaaggctctgctaccagatcttcaacttcttggatttgttcattcattacgtcacgaaccatttgaatccgctcttcaagggagacagcaagttcatctgcataagccatattaagaacgtggtagaaattttcttgtgcacttaaagcaccagataacaaataatcctgatcttcctctggtaaaagcatgtgttgctcatattcagcaaaagtgtttttgttaatctcaagcaaagcacccatcataacatcatactgataatctttatgaccaggattgtttaaaataacatcttcaaagatattaagaagaatcttagtaacaaaagcattgttttcggcaccttccatctgacggattaccgtgattacttctttttctttagcgttttggctcatagtgtttctcctctgattctatttggatataacccgtggttttgcacggattacactcgtagatttgacctacatccgccataacataccctgaaccaccacaataggggcaaggcataggaggacaaggtatggacgtagtaattgttacaacatcatcaattgctaacattttcgccattattaccactccgctattaatgtcagaccaagtgcatcgttaaaacgctcaacatttgaatataggtatggaggggttatttgctctaaaatttgcgctgcttccacttggtgctctatgttttgagaagttcgggcataaactaagacacccattttattcaaaataaaaacagaatcagacccatccagcttatcaacggctgcatacattttagtcatatcttctcctggattaccattcagcaacaagactaggattcatagcaaaagctttgaaagttgttatatcatgactaacattaaaactaacaccttttactttctctagtatttgaactttcgtctctccgtcccacggtaggatgataacaccatcttctgtgatcaaccaccaatggttctctccatctcgtgaatcttttgctttgtatagaaaattggaactcattcttcctcctgttcagtattattcacttgtgagacacaatgtaacacaagctttgtgagtttgtcaaggatctaattacattctacagaagaagagggttaataactataggaatagtcttactataggtgcaatactagttaagagttagattgataggtaagatttatcttaaacagataattgataggtgaatcagcattgactttttcaaaaaaatatggtaccctaaacatatgtttacgagcgatagcgagtagtagatttctatataataatgtttcatgttactcacgatagtgaggtatgaaacgtaagtgaaggtagataaactatgatcatctatctaccatctattaatcaatatagatctaatgttagactaacagttatccaacaaacaatagcagtccatataacaagtaatgatctacaaactacatcccattcaaatacattgtatgttaaccattctcccatcacaacagaagaacccaaccaacatccaacaacaactataatccaataaaagaatcctacagtagagtatacagcaaaagcttttaacatctttcctccagaagttgttcaacagtttgacgagtatgattgtcccagtaaaccttagtgaagtcaagagttccttgttcgtttctcttagtttcgaagaagtatccgccttctctttctactctgtctctaatgtgctctatgagatgctttttacaaggcgctgataaacaatagtcatactccccacgaagcaatgctatagctgcaagcctgttatcaccaacttgagatattgtccaagaaagcatactaccttctatacctaaaggtgtttgtttaattgaatatccaccataccgactgatagaaattatggcaagcggttctcctgttagagatcttcctaatgcaccttctgttggacttccttcaacaatacgtttgaaaaaagctttgatcttacctaacataagtcccccttaaaaatttacttgtttaaagtatctcttacttgttctgcttgacgtttagtaataaaagtcccaaccgaaacattaaactcattgactacagagaacgatccatcacgattctcacgaatattaaacaatttcttactcatttctacctccttcaatgctttgtgccgtaccatcccaagccattagaatgttttccaatagttcacgaggctctttaaaccaaacttccatttcatcaaggatctcgtccgtgtcgtaagtataacctggaacaccaagttcacatttcttaagatagctttctaaccagcggtgataattcttaaccccattcatactgtgctaccttttcttttacccaatctacacgatctttatcataatgttgagtgccaaggaaaagaccattgtggaaactttcctcaccgaaaggataaattggatctaaaccatcttcttcaaacaacatttttaatctgtttgatgtagatttcttagtttctgaattgtagtttgtggttataagccaatcttccaaacaacgacacaagccttgacgagaatcccaaggaaaaacacaatctccgacaggagcttgttccgcccatttcaaccaacctttaaaaaagttatacaatatgtagtcttcagtgtaatacattagcgcctcctttacatatacaataacaccccgcagaacggggtgcaactattatttaatctgtttaaaaatttcgtttggaacggtaccgtttaaaataccttctacgatctcttgtgcagatgtgtaagagagtccaaaagaagctacgcctaaaattgcctccttcaaaggatctggcaggggttcaaaattacctgcaacaaagccacctgcacaagcagcgccttcatcatcccagaaaacaactgctacatcgttaccatgcgaagttgtgcgatgtgcaataacttcaacttcggtgccttctttaggaatagaataagaactgtcccaagaatatctttccaaatcaggaacaaagataaccttttcacctactggcggcagttcacaacgataataccaagaatttttttcgttcattgttttattttcctccacgtcttcaaaagtaacgatccaatcgtagccgtaagaaaacggatattttagatctaaagaataagtttgacccagatcgtcctcaacaagaaccatcctatcaaagtttgcgatgttaggattatgtttaatgacagggtatgtttcccctacagtaaattgtcccaaatggtaaagttcatctctccaagtaatttggatatgagtgcattgaatcataaaatcacccccaagaaatatttatatatcttacaatgtttgcactgtctaaagccatgtttgcaccacacccacgctcgcgccagtaactaacaataccttcacagatactctcttgtatcttcttatcacttggcatttctagtataagacttgggttatggatgtttatgttataatatccttcctctgcggcttgagctaccaatttacttactttttcaaagtacggggaattagtggcatctgccattgtttttaaggtttgtctatccattattctgcctcatcatagaagaaatccaactcttgttccagttgtttaactcgatccttaacccactgtacacgtttttcattaaacatgcaaagtttttccttacactcttgatcaaaagattttgctgtgttaaatgggaaagttgtatctaagccagcgttttcaaaactggctaattgcataccataaacagaatatgctaaagaatgctctgcccgaaattcatcacttatatacatacgcagacctcggcataagccaacatcagcgtaaccactttctcttggaaagagacggaggtgttctaaccaattattatagaaatctaaaagaatcctcttacacttcaaatcgattgtcataaaaagtttctccttcatcaaacaagttggcaagattgttcacggcaatctgggtggaaagaagacaactgtttgcagacacataagcacgagctattttgtaaacataggctttcgtattacaattgtctaggatcggaagactttcgtcgatagcatctataacactaaacatcacatctaaaactttaatggtgagtgttgtatttgtatcttcgtaacctttttcaatccgacctgaatagaaagcatagtcctcaacagttcgtcgtttaaactggcgcataaaatcggaaccatctgtagaagaattgaacaaattttcaaaaaatacgtcccatccggcaaaaagggctgacagcatgattaagtgatctacagattcattttcttgtttcatatcctctcctatttaatgttcagtctggcacacaaacgagaaacagttttagcatcccatttaccaccacgtgctgttgctattttcttactattgaatttttcagcaatagttttgtaaggtgtatttaaatctctaaggatctccaagcgttcacggaatgtctctgcaaaagcgtctgctttaacagagttgtattccattttcccttttctacgtgcagcggcagttttaggattagcactgcctaataaaacacctttgctttttgctacagcaagaccctttttaatacgatcagcaatacctctaacttcttgttcagccagaacagacagaagctgtataaccattttatctgcgtctggcatgatggctatcttaacatcgatcatctctagaagagttgctacagaggcgttgttacgacccaagcggtcaactttagctaccaataaagttgctccggtagctttacacatttcaaccgctttaaccagttccttacgttgcttccaatcgcccttacccgaatagtattcagaaaattgaccaataacttccgcatcaggcacagagtctaagaagtaacggatatcattctcttggctctccataccatattgtgcctcaccttttttctgtttagaaagacgactgtaaacaacaaactttttcataaccactcctaaaccattgctgtattcccactttacaaaacgaataaacacttgtcaaggtatttattatttacagccattctactcttaagcagtattcctcgacatgttgtttacgcttgtttttgtctatgtaagtgtattcaacaaatgtaccaagatagtccttgtctacattgtgtgggctattgattggacatttagtacgacaaacccgttgccactgacgaagaattacggctttgtttttaggatcaaagggatgagggtaatgtaccttcattcacgcctcctgttttagtcgaaaaatattttgaataactagagatgtatagttatcacttagtcccataagttctacttccaagtttcgaatatcttccaccgtaggaggatatgttggtaaaaatgcttgagcattgccagatccaacttcattgctgtaagcagttgtaaaattgtaagatacaaaacatgaaacacctttttgtcggtacccgattaatcgacgaatacaccagataagagccaaacctatgagaacacctcctaaaccagctaacaagaaggataagttcataatagcttcgttaacaactgcttgcattcttttctccttagttatgtaaacccaaatgtgctacaacttctctacgataagataactgcatagcgattttatagccaggatgaaaaccatttacatcaatgtcatcaaaccaaatctcaaatgaaccgtgcttaagggtcattaagttgatataccattcgatagtataatgatcgtcagcaataccttcgatgtaaaattctcggcaacagtgagataggtcttcactcgtttcacgtttccagtctttgatagcacccttctctttggctctattcagaatctggatcaggtcttcaacttgcattttctctccttttgtggttaatctttatattttaaatattgcaccgttttctgtcaaatagcctctttctctaaaacaactctttggatcacaacatcttcaatgtttgccaaacaatgacccaaagtcaatcccgccttgaaaggtgcttgtagcttggattcggcttctacgcgaacagaatgttccgctccgcacatggtgtaatctacacggtaaacgtttaaacctaagaacatagattcttcttcctcacaacaaggattgagttcagcccagtcccaagcatctccttctaaagcaaatggagtgttgtaattgaagaaagtgcagcgcggatattccccgttgaaagaatctttcagcagaatatatttcttaccgtttgcatggaagagttctcctttgataaccttacctacaaggatatcactgttagtggaacgggtgcaaataaactttttcatgattctctcctctctgtttcgatgagatgattatgacaaagacagatccctctgtcaaccaattatttcttctgctacacgttcttttacccagttcacacgatccacattcaggtgacatacctctgcatctgattctaagacatatgcatcgtagttctcattgaagggtaattcttgactcaaccctttgtggtaaaactgtgcgtgcatttcatctgacaaggttgttataaggggattgtagccatccgcgtgcacatagtcgaataagttggcacacaaaccgcaatcccgccgaaaaggtttaccatcaggagcaccaacatcaatccatgctaaataaattttataaaactccatcaaccttgcgcttgttttcatttagccacctcgttagtttttcttgctctttgtttgcgcgttctaagcttctgaactttaaacagagggaacgagaatccctacccatgcacaagctttcatctaagctgtgctccccaaccaaccaaagagagtatgtttgaacatctaacccatagtttctgtctttccataatctcaccccaagaacatcaaaaggagcttccgcgactaaagaaagaaaccgatctttattcatattacaccaccttgctcacgttgataatgtatgtataccatacataaaataaatgtcaaggcttatcgacacagatagttgtaacgatctgctttatcctctgcttcagcaaagtcagcaatcaaggaagttttaagttcttccaactctttcacccaagagtcgtaatcgttctctgtttctccaatagtttggagaaaacaatcgtcgtagccaatagacatggctaactcgataatacgttccagtttttcttccatcacttctctccttctacagacttctgtttaacatagaagtaagcaccacgagcaagatacaaatcatgttcaatatcgctcacaggaaaaccatctggatttttaaaaccatcttctacagccaaaatgagagatcgtaaactgatagctaacaactccatttgatcttgtagttttctattttctgcataaaggtttttgcatttttgttctgcatttttccattgttctaatgcgtgatcatagtttcgcataagaatagctttttcttgttcacgtttaaaggtttcttcacaaatgaaacctgtgcctaaaccagtttcttcgtctttgacaaacacttcaacaaaatcttcagacatattatctcctatttgcttttggatactcccaaccctctgggagatcttctttacacatccactcaaaaggccagcgacaataactgtgccactttccatcgagatacaagcaatcttcatccactggtccacatgggttgataactacaacacacccttttgctcgccaatattgaactatttcagggtaaaaacaagaaacgtcggtccactcccaaatacgttcatcattcaaacggtatgctgctggcaatgtctctttcatattgttaaacctgaaccaaggaatcacgccaattaaggttgtttccttctcctaaataccagcaatctgcaacattaaaaacttcataaggaatacgccacgcataagcgaaatctggaatctccggtttatgagtgtaaccataacgctcgccacttgaatctgttgcaatataattaacaaattcaggaacatctaaagtttcaccttgatactctacacgcttgaagccatcttccacttctttctcaccttctaccaggcaagcaagaagtttgatgtgagggcaagtgaaatcttgtcctggataacgtgaacggatatcagttaacaatgcgattacagcatccgataactctttataatcctcatatcttacaaatgtaccttctgtcggatcatcaggacacaaacgccaagcatcgttgcagtttacagcgtaatgtctcattttagtcataaatcccccttatcgttgcgtacaatcagcttctagagtaagaacattcgttattgttttatcattttttagaaatgcaaaccaagctaaagtgtctcgatactctgtgcgcatcatcacaacttcagtagtgccaataaactgttgtgaaccatacatagcactaccatctttacttttttctcctgtgtattgcagaatgccactcatcaaagcaccacctgaatttttaaggataaaggagttatctttattaatgacaacagaagccattttcttaggaccagaaatcacttcacctttcagatttttaacaaccacatctgacacacactcataaacttgagacgcatttgctgttccgagtgatgcaatgagtaacaataaacctacgatgatctttttcatataaactctcctctttatttacccgacgcgcccctgttgggcggaaagggttgtacgcaaaatttttagctccaatcgtcgggtgtcccgactccttacgcttttaatgttttaacagaatgacaagctttgcaaaccataaccggaacaagttcgtctccactatccgcataaatcactgttttcttcattctgccaccacatgaacaatactttgtatgatccactggaatattaaatattccgtaatcactttcatactgtttcttacatatggacactgcttttgtaaaacaagacatacctactccgaaagaacattcacttcatcagagaaaaatacgtaatcacaaccttcccaaacatcttcttcaaaccactcccatccctcaactttggacatttcttctatcgtaacaaagaatgttgtcatgtcatcgctaacaagatgtgcttcaacaacaaaaggaatctgaacattctcaaaaccaacataacccaatacatcacaaactctaattttcatattatgctcctttgtagattatgttggtgcgttcacactctgccaccagcaagcgattagcaatgttgtccataggaacatcaatctctacactccaggcattgatacacacttcttcaatctttgctcccaacgtttgtgcgatcagggcaacagccccagcatattcaagtttaatctcaattaccattttgcacctcaaggttatttatcttgcttgtatcttacaaggcgatcacactctaggtcaagaaatttctccgcatttcgttcatgtttgagcacaatacattgttctttccagtctgtagctctttgctgcgctgctcctgggtcaaaatcccaaagccatgccgcgaaaagaacaaccataacaattactaagaaactaaacactattggcattgcacatcctcctgtgtagcatgatcttttacccattttatacgggcagcattcaaatgcaatagcccgtttctttgtgcaaaatcatatttaaaccagtctccgtcatcaaacggatattcttcagataatcctgcctcacggaattgcatttgcatttcataatttagatcatcataacctccagtccaaaggtagaggcaactacataatcctttatcatgccagaaaggattattatctggagcaccagctaacagccagttatgatatgcttgataaaattctttaagttcttctgacatttaacctcctgtatacagtgttgaaagatacgcagcaaaagcattctcggctgttttcacctcctcttcaagatcttcacgctctgctggcgttaatgcattccgcacagcagcgtctgcttctcggatttttcttactaaacgtgaaacatctttcagttgttgaaaagtagccatagtctttctccttttagtcaaaccagttgtattcagaagggtaacgagcgtagtttggcttgtaccccagccttttcatcttgacagacatacgagtgaagccaaaggttgttttgcccttagacataaacttcttcccactcttcattggcttcttagggtctttgatcgtctggtgtaacgctcgcgcaagccataaagtgtatgcgctcggcacttgattaactttcatcttgttacctttcatcatttcacctcggaccaatcaacagtgatccactcgtatgccccgttcagatccatttctttataaacaataagcccacgctcacgaagaattgctactgcttcatccatccacttcacctgattgtagatctcccctgggtgggtaggggaattatccagaccaaaatcagatggcgtgaaatttcttgaagagtttcccgctcgtgcttcagcaaaacatgtctcaatcacttcttcaacagggaagttgaaggaagcttcttttgcaatacggcgtaactgttcggtcagattcatcttattctcctttcgttgtgcgaaacattataacagatctaatgctttcaactcttctttcaatgattcaggcaggtcacattcaacgttaatcagagataacaagtgaccaacaatgccttccagatgttgatactcactataacttataaattcagaattgtaaacagaaacatccttagtttctttcacaacttcctccgcagacacgcacataaaaaagttacgctctccgcgagctaacaatatcccccaacttctgaacggatactgtccagcttcaataatatccccgctgtccaataaagtcaatgagcaaacagagcctgattcactaatatcttcgacgcggaaacaagacccttcaatcaaagctgccattttgaaattttcatcagagatcatagagaaagcttcacgggcagctttagttttgaaacagtaaacagcattaactttaaacataacattccccttttaaacaaaagatacgccgatggtagcaacaaccaaacgtttataaacttcaacctcttctgtaggattagccattttatgctctgctgcttctttcaaagcttcttcataactttcaaatccagaaatacaggtgtcatcagtcattacaacataccactcttcttttgttgtatcaacaggttcttcttggggttttccacgaccaccgcgaataccaccgtcaacaaaataatctatatgagcagaattgattatgtaacgatccccatcactagcagtgataataccgtctctccctatcacaacaattgacaagatattaccatttgcatcagtgctcaacacctcgaaaaaattctctccaatgatagcagcaatactgttatcaaaggtagaatatagtcgcatataatcctcatcctggaaacggtagacatgaccttctttaaacattgtatgctccttcttgttcaagagtaaaaggaatgatctcaccatagctgtattgcatggcatccacttctgcttctgcacgttgtgctagttctaatgttgcacagatcttgtaagggacagtgctgcattggctccagcctggggtattctttgttactaaatatacagtagttcctttattcataactttctcctcctagttcatcataccatttcaaagcgtcttttggaagcataccaaaatcttcttttagttctttttcaatctcttcttgcatttccatcagcgttttcagttgacggacttttttgtgcatagccacattcctttcattaaggtttgtcactacattatttgcacgactcggtggaaatgacaaaggtttcacgggaagataatcttcgatattatctagtgcgttacgaaaattatgatccaagaaacgcacatctacacgattgtaatcggcagctacaacagtgaacactttaaacggcacattggataaagtatgttgcacatagacagtacccatattgccttcaattttactattaacttccagcaaacctgtatgagcactaacataatcgtgaacacattgcacataagctgccagtatagtgaattgtctcattttctacctccccataatgctaacattttattgaaaatggaaacgcgtcgtgcgcgagatgccatatcataattcattctttccgtctctgcaagaaattgatctactgtaatcaacgcttttgtcttctctttcagagtttgcatattcatccattcacattcagagtaccaattatcgaaatacaaactatcaagaagatccattgctgcttcattctgttgtggtgtgatcattgcacacaacctttaataaacaatgttatattcgtgaggaaagaaaacataatcttccccttctcgtgcatctatctcgtcccaaaaccattcctgcacttccatcatcttcgagaatcgcacatgagcaagcccactgtattccaccatggcttccaacacaagtggaaattttgcatgtgccaatcctttataaccaccatcattctcaatacgaattttcatgcgtttcatgttatgcatccttcagacccttaatgaagtaacgactaaccggattccctcggaagtattcgttaatgaaatcgataacacacgtttcactgcctgtcacaacggacggcttacagttttggaattctacagctacacggctggctttagccagtgcttgcacacgtgtaggatcagtcaagaacattgtgacagttttcaaattcttcatttcatctcctcattggctatctgttatgtcatccattatacaaacatctttattcaatgcaagcattatttgcatcttcaattctgtctttcacccattgaagacgtaacgtgttggtatgacacgccaagtttatacactctgaaaggtaatccatgcaatctgcattgaacggaagattgtagtccaaccctgcacatgtaaattgtttcctcatttcatatttcaaacggtccaacaaactatcccgccatcttctatcttcttctctagttgtctcaccaacaaaatcgtttacataatctaccagacaaccacataagccactcgttgtgcgattcattgcaggatagttggcaatccatgctttatagaagcgaagcaatgttgcactgctcaacggaatactatttgtcatttttctctccttcgtaatcttgaattctatctagcacccattgtatacgatgtttgtttttatggcaaatatgttttttatgttcgaaggtgtattcctcataaccaccttcattaaacgggaaattgtaatccagtcctgcaccaataaactgtgatttcatctcctgttgtagacgaggtaacagttcatcaggatatgttccctctacatacttaatcaaacatatgcacaaaccactcgttgtgtgaccttctacctcaccagcaagccaagctttgtaaaacttcattagacgtaaactactcaacggaacatttttactctcttgcatgataatctcctctcatcgttagccatctttcatggcaatcatcaatcaccaacatcatacgatcaatcttagggaattacaatcgaattatcataggggattcctatcaattatcccatatataccgattatttttgacaccccactcttgtataaaaagtaacccaaaatatctacggggagagattatttcgggacaccctgtttcatggcaatcatggcaagaatatctaatagaatcaatatgatatggcaaaaatcatggcgcatacgtaagaagtgcttaa